(19) United States
(12) Patent Application Publication
(10) Pub. No.: US 2020/0253919 A1
(43) Pub. Date: Aug. 13, 2020

(54) TERPENE-ENRICHED CANNABINOID PRODUCT FOR WOMEN HEALTH

(71) Applicant: Buzzelet Development and Technologies Ltd., Or Akiva (IL)

(72) Inventors: Noa Raz, Gizo (IL); Aharon M. Eyal, Jerusalem (IL)

(21) Appl. No.: 16/726,841

(22) Filed: Dec. 25, 2019

Related U.S. Application Data
(63) Continuation-in-part of application No. PCT/IB2018/054783, filed on Jun. 27, 2018.
(60) Provisional application No. 62/525,835, filed on Jun. 28, 2017.
(60) Provisional application No. 62/588,394, filed on Nov. 19, 2017.

(51) Int. Cl. A61K 31/352; A61K 47/38; A61K 9/20; A61K 9/48; A61K 9/70; A61K 9/06; A61K 9/00; A61K 9/02

(52) U.S. Cl. CPC A61K 31/352; A61K 47/38; A61K 9/20; A61K 9/4808; A61K 9/0053; A61K 9/06; A61K 9/0073; A61K 9/02; A61K 9/7023

(57) ABSTRACT

A product for treating conditions and/or symptoms associated with women health is described, which product comprises at least one cannabinoid in a specific amount, a primary terpene in a specific amount, at least 5% by weight of a non-cannabinoid, non-terpene, carrier, optionally at least three secondary terpenes, and optionally at least one phytoestrogen; wherein said non-cannabinoid, non-terpene carrier comprises cellulose and the terpenes to cannabinoids weight/weight ratio in said product is about 0.1 to about 1.0. Also described is the product wherein said non-cannabinoid, non-terpene carrier comprises less than 5% by weight cellulose and the terpenes to cannabinoids weight/weight ratio in said compositions is about 0.05 to about 1.0, forming a terpene-enriched product. Also described are uses of the product for treating conditions and/or symptoms associated with perimenopause, menopause, female urogenital or reproductive system infections and/or disorders, menstrual cycle, hormonal deficiency and/or hormonal imbalance.

### CROSS-REFERENCE TO RELATED APPLICATIONS

The present application is a continuation-in-part of International Application No. PCT/IB2018/054783, filed on Jun. 27, 2018, which claims the benefit of priority of U.S. Provisional Application Ser. No. 62/525,825 filed Jun. 28, 2017 and U.S. Provisional Application Ser. No. 62/588,394 filed Nov. 19, 2017, and which are incorporated by reference herein in their entirety.

### FIELD OF THE INVENTION

The field of art to which this invention generally pertains is terpenes-enriched products, and specifically terpenes-enriched products for therapeutic use.

### BACKGROUND OF THE INVENTION

Menopause represents the transition of a woman from a reproductive to non-reproductive state as a result of a major reduction in female hormonal production by the ovaries. The transition is a natural process or may be the result of surgical intervention (removal of the ovaries) or the result of therapeutic regiment (e.g., administration of hormonal therapy for the treatment of patients with early stage breast cancer).

A diminished level of estrogen, the female hormone, has a direct effect on the hypothalamus, the part of the brain responsible for controlling appetite, sleep cycles, sex hormones, and body temperature.

Thus, menopause may be manifested by various conditions and/or symptoms, including hot flashes, facial flushing, night sweat, osteoporosis, fatigue, irritability, insomnia and sleep disorder, inability to concentrate, depression, memory loss, headache, anxiety, nervousness, muscle tension, myalgia, intermittent dizziness, paresthesia, indigestion, palpitations, tachycardia, nausea, constipation, diarrhea, arthralgia and/or arthritis, pelvic pain, spasms, cramps, paresthesia, Polycystic Ovary Syndrome (POCS), premenstrual syndrome (PMS), premenstrual tension, premenstrual dysphoric disorder (PMDD), cold hands and feet, weight gain, mental awareness problems, sexual pain disorder, dyspareunia breast pain and/or mastalgia, menopausal skin, inflammation, oxidative stress, cardiovascular disease, loss of pelvic muscle tone, urinary incontinence, vaginal dryness, vaginal infections, human papillomavirus (HPV), Uterine Fibroid, cystitis, women hormonal irregularity, irregular periods, reduced libido, hair loss and/or thinning, age-related winkles, brittle nails, gam disorders, irregular body odor. Menopausal conditions and/or symptoms affect about 70% of women approaching menopause. Typical menopause conditions and/or symptoms last for the whole menopause transition, but some women may experience them for the rest of their lives.

Hormone therapies such as estrogen therapy, progesterone therapy or estrogen-progesterone combinations have proven most effective. However, there are definite risks to hormone therapy such as increased risk of breast cancer, stroke and heart disease. Furthermore, women who have or previously had breast cancer, ovarian cancer, endometrial cancer, blood clots in the legs or lungs, stroke, liver disease, or unexplained vaginal bleeding should not receive hormone therapy. Thus, it would be highly desirable to provide a treatment for improving menopausal related conditions and/or symptoms which do not cause debilitating side effects.

Cannabis is a complex plant comprising over 400 chemical entities of which more than 60 are cannabinoid compounds, some of which have opposing pharmacological effects. In the naturally occurring Cannabis plant, cannabinoids are synthesized and accumulated as cannabinoid acids (e.g. tetrahydrocannabinolic acid (THCa) and cannabidioolic acid (CBDA). When the herbal product is dried, stored and heated, the acids decarboxylize into their active forms, such as tetrahydrocannabinol (THC) and Cannabidiol (CBD) [De Meijer et al. 2003; The inheritance of chemical phenotype in Cannabis sativa L. Genetics 63: 335-346].

There are major differences in functional pharmaceutical effects between the acid and decarboxylized forms (for instance THC is psychoactive, while THCa is not psychoactive), in physical properties (such as solubility and boiling point) and in chemical properties (e.g. THCa may undergo esterification and dimerization, while THC cannot, THCa solubility is pH dependent, while that of THC is not, etc.) (e.g. De Zeeuw, Journal of Pharmacy and Pharmacology, 1972; Mechoulam, Naturwissenschaften, 1978; Grotenhermen, Clinical Pharmacokinetics, 2003).

Terpenes are the major components of the essential oils present in various Cannabis varieties. These compounds are responsible for the distinctive aromas and flavors. Terpene yield and distribution in the plant vary according to numerous parameters, such as environmental conditions or maturity of the plant. (Meier and Mediavilla, 1998; Brenneisen, 2007). Terpene yield is less than 1% in most cannabis asays (Potter, 2009). Monoterpenes usually predominate in the natural plant (limonene, myrcene, pinene), but these headspace volatiles (Hood et al., 1973) suffer diminished yields with drying and storage (Turner et al., 1980; Ross and ElSohly, 1996), resulting in a higher relative proportion of sesquiterpenoids in processed cannabis. Being volatile agents, terpenes further evaporate during heating and decarboxyalation.

Processing cannabis plant material typically leads to terpenes loss so that most of cannabis products are of relatively low terpene content. That is particularly true for cannabis extracts and products thereof, such as cannabis tablets, cannabis gel capsules, cannabis patches, cannabis suppositories, etc. Most cannabis terpenes boiling points are in the range between about 1500° C. and about 220° C. and they evaporate, at least partially, during cannabis buds drying, during solvent separation from extracts and during decarboxylation. Monoterpenes are lost at a rate greater than that of terpenes with a higher molecular weight and terpenes carrying no hydroxyl groups are lost at a rate greater than that of terpenes that do carry hydroxyl groups. Primary terpenes found in cannabis include pinene, linalool, myrcene, limonene, ocimene, terpinolene, terpineol, caryophyllene, valencene, geraniol and humulene. Secondary terpenes include Phellandrene, terpinene, carene, fenchol, bisabolol, borneol, phytol, camphene, sabinene, camphor,

### SUMMARY OF THE INVENTION

According to an aspect of some embodiments of the present invention, provided is a product for treating conditions and/or symptoms associated with women health comprising (i) at least one cannabinoid in a specific amount, (ii) a primary terpene in a specific amount, (iii) at least 5% by weight of a non-cannabinoid, non-terpene, carrier, (iv) optionally at least three secondary terpenes, (v) optionally at least one phytoestrogen; and (a) wherein the non-cannabinoid, non-terpene carrier comprises cellulose and the terpenes to cannabinoids weight/weight ratio in the product is about 0.1 to about 1.0, or (b) wherein the non-cannabinoid, non-terpene carrier comprises less than 5% by weight cellulose and the terpenes to cannabinoids weight/weight ratio in the product is 0.05 to about 1.0, forming a terpene-enriched product with an enhanced therapeutic effect in treating conditions and/or symptoms associated with perimenopause, menopause, female urogenital or reproductive system infections and/or disorders, menstrual cycle, hormonal deficiency and/or hormonal imbalance, compared with that of a product comprising the same cannabinoids amounts and one half the amount of the primary terpene.

According to an aspect of some embodiments of the present invention, provided is a method for achieving a therapeutic effect in treating conditions and/or symptoms associated with perimenopause, menopause, female urogenital or reproductive system infections and/or disorders, menstrual cycle, hormonal deficiency and/or hormonal imbalance comprising administering to a subject in need a therapeutically effective amount of a product comprising (a) at least one cannabinoid in a specific amount, (b) a primary terpene in a specific amount, (c) at least 5% by weight of a non-cannabinoid, non-terpene carrier, (d) optionally at least three secondary terpenes, and (e) optionally at least one phytoestrogen, optionally combined with at least one herbal extract.

According to an additional aspect of some embodiments of the present invention, provided is a method for achieving, reaching and/or gaining a therapeutic effect in treating conditions and/or symptoms associated with perimenopause, menopause, female urogenital or reproductive system infections and/or disorders, menstrual cycle, hormonal deficiency and/or hormonal imbalance comprising administering to a subject in need a therapeutically effective amount of a composition comprising (i) a primary terpene in a specific amount; (ii) optionally at least three secondary terpenes; (iii) optionally at least one cannabinoid in a specific amount; and (iv) optionally at least one phytoestrogen. According to an embodiment, the composition comprises at least 5% by weight of a non-cannabinoid, non-terpene carrier.

According to an additional aspect of some embodiments of the present invention, provided is a method for treating women health comprising administering to the subject a product selected from the group consisting of tablets, gel capsules, medical patches, topicals, creams, varnishes, sublingual oils, sprays, edibles, suppositories, tampons, rectal candles, cigarettes, vaporizer liquids, nasal preparations, preparations containing micro and/or nano-emulsions, preparations containing micro and/or nano-particles and combinations thereof.

According to an additional aspect of some embodiments of the present invention, provided is a composition comprising five or less terpenes in a concentration of at least 10% of the total terpene content, forming a terpene composition with enhanced therapeutic effect for treating conditions and/or symptoms associated with perimenopause, menopause, female urogenital or reproductive system infections and/or disorders, menstrual cycle, hormonal deficiency and/or hormonal imbalance.

### DETAILED DESCRIPTION OF THE INVENTION

Unless indicated otherwise the term “menopausal” as used herein refers to peri-menopausal, menopausal, or post-menopausal.

As used herein the term “treat” and/or “treating” refers to treating, healing, relieving, decreasing, preventing and/or reducing the risk of.

Unless indicated otherwise, percent is weight percent and ratio is weight/weight ratio. Unless indicated otherwise, weight ratio means the ratio between weight content, e.g. in an aqueous solution containing 20% solute and 80% water, the solute to water weight ratio is 20:80 or 1:4.

Unless indicated otherwise, the term “cannabinoid” refers to a compound that affects the endocannabinoid system. Cannabinoids are agonists or antagonists to receptors in the endocannabinoid system.

Unless indicated otherwise, the term “female reproductive system” refers to at least one of ovaries, fallopian tubes, uterus, vagina, vulva, mammary glands and/or breasts. Unless indicated otherwise, reference to the female reproductive system refers to organs that are involved in the production and transportation of gametes and the production of sex hormones.

Unless indicated otherwise, the term “female urogential system” refers to at least one of kidneys and ureters, bladder, urethra, uterus, fallopian tubes, ovaries, and/or vagina.

Unless indicated otherwise, the terms “premenstrual syndrome (PMS)” and “premenstrual dysphoric disorder (PMDD)” refer to symptoms selected from the group consisting of bloating, swollen painful breasts, fatigue, constipation, headaches, clumsiness, anger, anxiety, confusion, mood swings, tension, depression, inability to concentrate and combinations thereof.

Unless indicated otherwise, the terms “hormonal deficiency/hormonal imbalance/hormonal balancing” refer to abnormal levels of estrogen and/or progesterone. Unless indicated otherwise, conditions and/or symptoms associated with female hormonal disfunction and/or imbalance include impaired fertility, Polycystic Ovary Syndrome (POCS), amenorrhea, oligomenorrhea, hypermenorrhea, metrorrhagia, premenstrual syndrome (PMS) and premenstrual dysphoric disorder (PMDD).

Unless indicated otherwise, conditions and/or symptoms associated with perimenopause and/or menopause include hot flashes, facial flushing, night sweat, osteoporosis, fatigue, irritability, insomnia and/or sleep disorder, difficulty in concentration, depression, memory loss, brain fog, headache, migraine, anxiety, nervousness, restless, stress, mood swing, affect disorder, general discomfort, muscle tension, myalgia, intermittent dizziness, paresthesia, indigestion, digestive problems, intestinal disorders, abdominal pain, irritability, palpitations, tachycardia, nausea, constipation, diarrhea, arthralgia and/or arthritis, cold hands and feet, weight gain, mental awareness problems, sexual pain disorder, dyspareunia, breast pain and/or mastalgia, menopausal skin, inflammation, oxidative stress, cardiovascular disease, loss of pelvic muscle tone, urinary incontinence, vaginal dryness, reduced libido, reduced sexual dysfunction, hair loss and/or thinning, age-related winkles, stretch marks, brittle nails, gam disorders, irregular body odor, conditions and/or symptoms induced by estrogen deficiency and combinations thereof.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an embodiment, provided is a product for treating conditions and/or symptoms associated with women health comprising (i) at least one cannabinoid in a specific amount, (ii) a primary terpene in a specific amount, (iii) at least 5% by weight of a non-cannabinoid, non-terpene, carrier, (iv) optionally at least three secondary terpenes, (v) optionally at least one phytoestrogen; and (a) wherein the non-cannabinoid, non-terpene carrier comprises cellulose and the terpenes to cannabinoids weight/weight ratio in the product is about 0.1 to about 1.0, or (b) wherein the non-cannabinoid, non-terpene carrier comprises less than 5% by weight cellulose and the terpenes to cannabinoids weight/weight ratio in the product is 0.05 to about 1.0, forming a terpene-enriched cannabinoid product with an enhanced therapeutic effect in treating conditions and/or symptoms associated with perimenopause, menopause, female urogenital or reproductive system infections and/or disorders, menstrual cycle, hormonal deficiency and/or hormonal imbalance, compared with that of a product comprising the same cannabinoids amounts and one half the amount of said primary terpene. According to an embodiment, said product is a composition or the terms “product” and “composition are used interchangeably. According to an embodiment, said product comprises a composition comprising (i) at least one cannabinoid in a specific amount, (ii) a primary terpene in a specific amount, (iii) at least 5% by weight of a non-cannabinoid, non-terpene, carrier, (iv) optionally at least three secondary terpenes, (v) optionally at least one phytoestrogen; and (a) wherein the non-cannabinoid, non-terpene carrier comprises cellulose and the terpenes to cannabinoids weight/weight ratio in the composition is about 0.1 to about 1.0, or (b) wherein the non-cannabinoid, non-terpene carrier comprises less than 5% by weight cellulose and the terpenes to cannabinoids weight/weight ratio in the composition is 0.05 to about 1.0. According to an embodiment, said product comprises a composition resulting from processing a plant material.

According to an embodiment, the enhanced therapeutic effect comprises a shortened onset time, increased magnitude, extended duration, reduced dosages, reduced secondary adverse symptoms, reduce frequency of conditions and/or symptoms, reduce severity of conditions and/or symptoms, reduce consumption of other drugs and combinations thereof.

According to an embodiment, the conditions and/or symptoms are selected from the group consisting of hot flashes, facial flushing, night sweat, osteoporosis, fatigue, irritability, insomnia and/or sleep disorder, difficulty in concentration, depression, memory loss, brain fog, headache, migraine, anxiety, nervousness, restless, stress, mood swing, affect disorder, general discomfort, muscle tension, myalgia, intermittent dizziness, paresthesia, indigestion, digestive problems, intestinal disorders, abdominal pain, irritability, palpitations, tachycardia, nausea, constipation, diarrhea, arthralgia and/or arthritis, dysmenorrheal, pelvic pain, spasms, cramps, labor contraction, endometriosis Polycystic Ovary Syndrome (POCS), amenorrhea, oligomenorrhea, hypermenorrhea, metrorrhagia, premenstrual syndrome (PMS), premenstrual tension, premenstrual dysphoric disorder (PMDD), cold hands and feet, weight gain, mental awareness problems, sexual pain disorder, dyspareunia, breast pain and/or mastalgia, menopausal skin, inflammation, oxidative stress, cardiovascular disease, loss of pelvic muscle tone, urinary incontinence, lower urinary tract disorders, vaginal dryness, women urogenital and/or reproductive system infection, fibroid, myomas and/or cysts in the female genital system, human papillomavirus (HPV), Uterine Fibroid, cystitis, urethritis, pyelonephritis, women hormonal dysfunction and/or imbalance, irregular periods, pre-eclampsia toxemia, reduced libido, reduced sexual dysfunction, hair loss and/or thinning, age-related winkles, stretch marks, brittle nails, gam disorders, irregular body odor, conditions and/or symptoms induced by estrogen deficiency and combinations thereof.

According to an embodiment, the product comprises at least two cannabinoids, at least three, at least four or at least five. According to an embodiment, the content of each cannabinoid in the product is at least 10 parts per million (ppm). As known in the art, cannabinoids have an acid form and a non-acid form (which is also referred to as decarboxylated form, since it can be generated by decarboxylating the acid form). The acid form is indicated herein by the letter (a) at the end of the cannabinoid acronym, e.g. tetrahydrocannabiniolic acid is indicated as THCa, while the decarboxylated form is THC. According to an embodiment, the cannabinoids are selected from the group consisting of tetrahydrocannabiniol in acid or decarboxylated form (THCa or THC, respectively), cannabidiol in acid or decarboxylated form (CBDa or CBD, respectively), cannabigerol in acid or decarboxylated form (CBGa or CBG, respectively), cannabichromene in acid or decarboxylated form (CBCa or CBC, respectively) tetrahydrocannabivarin in acid or decarboxylated form (THCVa or THCV, respectively), Cannabidivarin in acid or decarboxylated form (CBDVa or CBDV respectively), Cannabinol in acid or decarboxylated form (CBNa or CBN, respectively), Cannabicyclol in acid or decarboxylated form (CBLa or CBL, respectively). As used herein, the term THC refers to THCa (tetrahydrocannabiniolic acid) and/or to THC (tetrahydrocannabiniol) unless indicated otherwise. As used herein, the term CBD refers to CBDa (cannabidiolic acid) and/or to CBD (cannabidiol) unless indicated otherwise. As used herein, the term CBG refers to CBGa (cannabigerolic acid) and/or to CBG (cannabigerol) unless indicated otherwise. As used herein, the term CBN refers to CBNa (Cannabinolic acid) and/or to CBN (cannabinol) unless indicated otherwise. As used herein, the term CBC refers to CBCa (cannabichromenic acid) and/or to CBC (Cannabichromene) unless indicated otherwise. As used herein, the term CBL refers to CBLa (Cannabicycol acid) and/or to CBL (Cannabicyclol) unless indicated otherwise. As used herein, the term THCV refers to THCVa (tetrahydrocannabivarin acid) and/or to THCV (tetrahydrocannabivarin) unless indicated otherwise. As used herein, the term CBDV refers to CBDVa (cannabigerovarin acid) and/or to CBDV (cannabidivarin) unless indicated otherwise. Thus, the term “CBD to THC ratio” may mean “CBD to THC ratio”, “CBDa to THC ratio”, “CBD to THCa ratio”, “CBDa to THCa ratio”, “CBD to THC+THCa ratio”, “CBDa to THC+THCa ratio”, “CBD+CBDa to THC ratio”, “CBD+CBDa to THCa ratio” or “CBD+CBDa to THC+THCa ratio”.

According to an embodiment, at least one of the cannabinoids is in acid form. According to an embodiment, at least one of the cannabinoid is at least partially in decarboxylated form. According to an embodiment, at least 50% of the cannabinoid is in decarboxylated form, at least 60%, at least 70%, at least 80% or at least 90%.

According to an embodiment, the product comprises THC and/or THCa. According to an embodiment, the product comprises CBD and/or CBDa. According to an embodiment, the product comprises THC and/or THCa at a content of less than 1%, less than 0.8%, less than 0.6%, less than 0.4% or less than 0.2%. According to an embodiment, the product comprises both CBD and/or CBDa and THC and/or THCa and the weight/weight ratio between CBD and/or CBDa and THC and/or THCa ((CBD+CBDa)/(THC+THCa)) is at least 10, at least 15, at least 20, at least 25 or at least 30. According to an embodiment, the product comprises CBG and/or CBGa. According to an embodiment, the product comprises CBN and/or CBNa. According to an embodiment, the product comprises CBC and/or CBCa. According to an embodiment, the product comprises CBL and/or CBLa. According to an embodiment, the product comprises THCV and/or THCVa. According to an embodiment, the product comprises CBDV and/or CBDVa.

According to an embodiment, the product comprises at least 5% by weight carrier, at least 10%, at least 15%, at least 20%, at least 25%, at least 30%, at least 35% or at least 40% by weight. Any compound other than cannabinoids and terpenes is a suitable carrier. According to an embodiment, the carrier is selected from the group consisting of vegetable oils, e.g. coconut oil, olive oil or sesame oil, pharmaceutical excipients, honey, bees wax, cellulose and combinations thereof. As used herein, the term cellulose refers to cellulose, hemicellulose and their combinations.

According to an embodiment, the product comprises at least one phytoestrogen. According to an embodiment, the product comprises at least two, at least three, at least four, or at least five phytoestrogens.

According to an embodiment, the product comprises a primary terpene and optionally at least three secondary terpenes, at least four or at least five. The term “terpene”, as used herein, refers to both terpenes and terpenoids. As used here, the term “primary terpene” refers to a terpene that forms at least 20% by weight of the total amount of terpenes in the product, at least 30%, at least 40%, at least 50%, at least 60%, at least 70% or at least 80%. According to an embodiment, the product comprises multiple (e.g. two or three) terpenes, each one of which forms at least 20% by weight of the total amount of terpenes in the product and each one of these terpenes is considered a primary terpene. As used here, the term “secondary terpene” refers to a terpene that forms at least 10 parts per million (ppm) of the product. According to an embodiment, the content of the primary terpene in the product is at least 2 times greater than that of any secondary terpene, at least 4, at least 6, at least 8, at least 10, at least 15, at least 20, at least 25, or at least 30 times greater.

As used herein, “terpenes/cannabinoids (or terpenes to cannabinoids) weight/weight ratio” means the weight ratio between the combined amount of terpenes and the combined amount of cannabinoids. According to an embodiment, the non-cannabinoid, non-terpene carrier comprises cellulose and the terpenes to cannabinoids weight/weight ratio in the product is about 0.1 to about 1.0. According to an embodiment said non-cannabinoid, non-terpene, carrier comprises at least 5% by weight cellulose, at least 6%, at least 8%, at least 10%, at least 15%, or at least 20%. According to an embodiment, the ratio is greater than 0.1, greater than 0.15, greater than 0.2, greater than 0.25, greater than 0.3, greater than 0.35, greater than 0.4, greater than 0.45, greater than 0.5, greater than 0.55, greater than 0.6, greater than 0.65, greater than 0.7, greater than 0.75, greater than 0.8, greater than 0.85, greater than 0.9, greater than 0.95, greater than 1, greater than 1.2, greater than 1.5, greater than 2.0, greater than 3 or greater than 5. According to an embodiment, the ratio is less than 0.9, less than 0.8, less than 0.7, less than 0.6, less than 0.5, less than 0.4, less than 0.3, less than 0.2 or less than 0.15. According to an embodiment, the product comprising more than 5% cellulose is selected from the group consisting of cannabis plant material, e.g. cannabis buds or cannabis trim, ground forms thereof, plant material preparation for vaporizers and cannabis cigarette.

According to another embodiment, the non-cannabinoid, non-terpene carrier comprises less than 5% by weight cellulose and terpenes to cannabinoids weight/weight ratio in the product is in the range between about 0.05 and about 1.0. According to an embodiment said non-cannabinoid, non-terpene, carrier comprises less than 5% by weight cellulose, less than 4%, less than 3%, less than 2%, or less than 1% cellulose. According to an embodiment, the ratio is greater than 0.1, greater than 0.15, greater than 0.2, greater than 0.25, greater than 0.3, greater than 0.35, greater than 0.4, greater than 0.45, greater than 0.5, greater than 0.55, greater than 0.6, greater than 0.65, greater than 0.7, greater than 0.75, greater than 0.8, greater than 0.85, greater than 0.9, greater than 0.95, greater than 1, greater than 1.2, greater than 1.5, greater than 2.0, greater than 3 or greater than 5. According to an embodiment, the ratio is less than 0.9, less than 0.8, less than 0.7, less than 0.6, less than 0.5, less than 0.4, less than 0.3, less than 0.2, less than 0.15 or less than 0.1. According to an embodiment, the product comprising less than 5% cellulose is selected from the group consisting of cannabis trichomes, cannabis extracts and products thereof, such as tablets, gel capsules, medical patches, topicals, creams, varnishes, sublingual oils, sprays, edibles, suppositories, tampons, rectal candles, cigarettes, vaporizer liquids nasal preparation, preparations containing micro and nano-emulsions, preparations containing micro and nano-particles and combinations thereof.

According to an embodiment, the primary terpene, the secondary terpenes or both are selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thujone, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol, isomers thereof and combinations thereof. According to an embodiment, the primary terpene the secondary terpenes or both, are selected from the group consisting of pinene, limonene, linalool, caryophyllene, myrcene, terpineol, eucalyptol, geraniol and combinations thereof. According to an embodiment, the primary terpene the secondary terpenes or both, are selected from the group consisting of pinene, limonene, linalool, caryophyllene, myrcene, eucalyptol and combinations thereof. According to an embodiment, at least one of the terpenes is a-cyclic. According to an embodiment, at least one of the terpenes is cyclic. According to an embodiment, at least one of the terpenes is not found in cannabis buds or is present there at less than 0.2%, less than 0.1%, less than 0.05% or less than 0.02%. Such terpene is referred to as “non-cannabis terpene”. According to an embodiment, the terpene-enriched cannabinoid product comprises the non-cannabis terpene at a concentration of at least 0.2%, at least 0.5%, least 0.8%, at least 1%, least 1.5%, at least 2%, at least 3%, least 4%, at least 5%, at least 8% or at least 12%.

According to an embodiment, the primary terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thujone, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol and combinations thereof. According to an embodiment, the primary terpene is selected from the group consisting of pinene, limonene, linalool, caryophyllene, myrcene, terpineol, eucalyptol, geraniol and combinations thereof. According to an embodiment, the primary terpene is selected from the group consisting of pinene, limonene, linalool, caryophyllene, myrcene, eucalyptol and combinations thereof. According to an embodiment, the primary terpene is a non-cannabis terpene.

According to an embodiment, the terpenes comprise at least one monoterpene selected from the group consisting of limonene, myrcene, pinene, linalool, geraniol, terpinene camphene and isomers thereof. According to an embodiment, the terpenes comprise at least one sesquiterpene selected from the group consisting of nerolidol, caryophyllene, farnesene, zingiberene, vetivazulene, guaiazulene, longifolene, copaene, patchoulol humulene and isomers thereof. According to an embodiment, the terpenes comprise at least one diterpene selected from the group consisting of phytol, retinal, retinol, phytane, cembrene, sclarene, labdane, abietane, texadiene, stemarene, stemoden and isomers thereof. According to an embodiment, the terpenes comprise at least one hydroxy-terpene selected from the group consisting of nerolidol, geraniol, linalool, phytol and isomers thereof. As used herein “hydroxy-terpene” refers to a terpene carrying a hydroxyl function.

According to an embodiment, at least one of the terpenes is a monoterpene, at least one of the terpenes is a sesquiterpene and the monoterpenes to sesquiterpenes weight/weight ratio (i.e. the weight ratio between the total amount of monoterpenes and the total amount of the sesquiterpenes) is greater than 1.5 greater than 2, greater than 2.5, greater than 3, greater than 3.5, greater than 4, greater than 4.5, greater than 5, greater than 6, greater than 7, greater than 8, greater than 9, greater than 10, greater than 15, or greater than 20.

According to an embodiment, at least one of the terpenes is a monoterpene, at least one of the terpenes is a diterpene and the monoterpenes to diterpenes weight/weight ratio (i.e. the weight ratio between the total amount of monoterpenes and the total amount of the diterpenes) is greater than 5, greater than 6, greater than 7, greater than 8, greater than 9, greater than 10, greater than 12, greater than 14, greater than 16, greater than 18, greater than 20, greater than 25, or greater than 30.

According to an embodiment, at least one of the terpenes carries no hydroxyl group, at least one of the terpenes carries hydroxyl group and the non-hydroxy-terpenes to hydroxyl-terpenes weight/weight ratio (i.e. the weight ratio between the total amount of non-hydroxy-terpenes and the total amount of the hydroxy-terpenes) is greater than 1.5, greater than 2, greater than 2.5, greater than 3, greater than 3.5, greater than 4, greater than 4.5, greater than 5, greater than 6, greater than 7, greater than 8, greater than 9, greater than 10, greater than 15, or greater than 20.

According to an embodiment, terpenes form at least 0.5% by weight of the product, at least 1%, at least 2%, at least 3%, at least 5%, at least 10%, at least 20%, at least 30%, at least 40%, at least 50%, at least 60% or at least 70%.

According to an embodiment, the product comprises a composition, which is liquid at 300° C. According to an embodiment, the product comprises a composition, which is a suspension at 30° C. According to an embodiment, the product comprises a composition, which is essentially clear of haze or suspended solids at 300° C.

According to an embodiment, the product comprises cannabis plant material. According to an embodiment, the product comprises cannabis bud. According to an embodiment, the cannabis bud forms at least 20% of the product, at least 30%, at least 40%, at least 50%, at least 60%, at least 70% or at least 80%.

According to an embodiment, the non-cannabinoid, non-terpene, carrier comprises cellulose, and the product comprises (a) tetrahydrocannabinol (THC) and/or tetrahydrocannabinolic acid (THCa) in a total concentration of at least 5% by weight; (b) cannabidiol (CBD) and/or cannabidiolic acid (CBDa) in a total concentration of at least 5% by weight; (c) tetrahydrocannabinol (THC) and/or tetrahydrocannabinolic acid (THCa) in a total concentration of at least 2.5% by weight, and cannabidiol (CBD) and/or cannabidiolic acid (CBDa) in a total concentration of at least 2.5% by weight; or (d) at least one of cannabigerol (CBG), cannabinol (CBN), cannabichromene (CBC), and cannabicyclol (CBL), tetrahydrocannabivarin (THCV), cannabidivarin (CBDV), and non-decarboxylated form thereof in a concentration of at least 0.5% by weight; and (i) the primary terpene forms at least 40% by weight of the total terpene content, and (ii) the primary terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thujone, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

According to an embodiment, the non-cannabinoid, non-terpene, carrier comprises cellulose, and the product comprises (a) tetrahydrocannabinol (THC) and/or tetrahydrocannabinolic acid (THCa) in a total concentration of at least 5% by weight; (b) cannabidiol (CBD) and/or cannabidiolic acid (CBDa) in a total concentration of at least 5% by weight; or (c) tetrahydrocannabinol (THC) and/or tetrahydrocannabinolic acid (THCa) in a total concentration of at least 2.5% by weight, and cannabidiol (CBD) and/or cannabidiolic acid (CBDa) in a total concentration of at least 2.5% by weight; and optionally (d) at least one of cannabigerol (CBG), cannabinol (CBN), cannabichromene (CBC), and cannabicyclol (CBL), tetrahydrocannabivarin (THCV), cannabidivarin (CBDV), and non-decarboxylated form thereof in a concentration of at least 0.5% by weight; wherein (i) the primary terpene forms at least 40% by weight of the total terpene content, and (ii) the primary terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thujone, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

According to a related embodiment said non-cannabinoid, non-terpene, carrier comprises at least 5% by weight cellulose, at least 6%, at least 8%, at least 10%, at least 15%, or at least 20%.

According to a related embodiment said tetrahydrocannabinol (THC) and/or tetrahydrocannabinolic acid (THCa) is in a total concentration of at least 4% by weight, at least 5%, at least 6%, at least 8%, at least 10%, at least 12%, at least 14%, at least 16%, at least 18%, or at least 20% by weight.

According to a related embodiment said cannabidiol (CBD) and/or cannabidiolic acid (CBDa) is in a total concentration of at least 4% by weight, at least 5%, at least 6%, at least 8%, at least 10%, at least 12%, at least 14%, at least 16%, at least 18% or at least 20% by weight.

According to a related embodiment, said product comprises tetrahydrocannabinol (THC) and/or tetrahydrocannabinolic acid (THCa) in a total concentration of at least 2.5% by weight, and cannabidiol (CBD) and/or cannabidiolic acid (CBDa) in a total concentration of at least 2.5% by weight; tetrahydrocannabinol (THC) and/or tetrahydrocannabinolic acid (THCa) in a total concentration of at least 3% by weight, and cannabidiol (CBD) and/or cannabidiolic acid (CBDa) in a total concentration of at least 3% by weight; tetrahydrocannabinol (THC) and/or tetrahydrocannabinolic acid (THCa) in a total concentration of at least 4% by weight, and cannabidiol (CBD) and/or cannabidiolic acid (CBDa) in a total concentration of at least 4% by weight; tetrahydrocannabinol (THC) and/or tetrahydrocannabinolic acid (THCa) in a total concentration of at least 5% by weight, and cannabidiol (CBD) and/or cannabidiolic acid (CBDa) in a total concentration of at least 5% by weight; tetrahydrocannabinol (THC) and/or tetrahydrocannabinolic acid (THCa) in a total concentration of at least 8% by weight, and cannabidiol (CBD) and/or cannabidiolic acid (CBDa) in a total concentration of at least 8% by weight; tetrahydrocannabinol (THC) and/or tetrahydrocannabinolic acid (THCa) in a total concentration of at least 10% by weight, and cannabidiol (CBD) and/or cannabidiolic acid (CBDa) in a total concentration of at least 10% by weight.

According to a related embodiment, said product comprises cannabigerol (CBG) and/or cannabigerol acid (CBGa) in a total concentration of at least 0.1% by weight, at least 0.5%, at least 1%, at least 2%, at least 3%, at least 4%, or at least 5% by weight. According to a related embodiment, said product comprises cannabinol (CBN) and/or cannabinol acid (CBNa) in a total concentration of at least 0.1% by weight, at least 0.5%, at least 1%, at least 2%, at least 3%, at least 4%, or at least 5% by weight. According to a related embodiment, said product comprises cannabichromene (CBC) and/or cannabichromenic acid (CBCa) in a total concentration of at least 0.1% by weight, at least 0.5%, at least 1%, at least 2%, at least 3%, at least 4%, or at least 5% by weight. According to a related embodiment, said product comprises cannabicyclol (CBL) and/or cannabicyclol acid (CBLa) in a total concentration at least 0.1% by weight, at least 0.5%, at least 1%, at least 2%, at least 3%, at least 4%, or at least 5% by weight. According to a related embodiment, said product comprises tetrahydrocannabivarin (THCV), and/or tetrahydrocannabivarin acid (THCVA) in a total concentration at least 0.1% by weight, at least 0.5%, at least 1%, at least 2%, at least 3%, at least 4%, or at least 5% by weight. According to a related embodiment, said product comprises cannabidivarin (CBDV) and/or cannabigerovarin acid (CBGVA) in a total concentration at least 0.1% by weight, at least 0.5%, at least 1%, at least 2%, at least 3%, at least 4%, or at least 5% by weight.

According to a related embodiment, said primary terpene forms at least 20% by weight of the total terpene content, at least 30%, at least 40%, at least 50%, at least 60%, at least 70%, at least 80% or at least 90% by weight of the total terpene content. According to a related embodiment, said primary terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thujone, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol and combinations thereof. According to a related embodiment, said product comprises a dried cannabis plant material. According to a related embodiment, said product comprises ground cannabis plant material, is a vaporizer cannabis filling material or is a cannabis cigarette.

According to an embodiment, the product comprises (a) at least one cannabinoid from the group consisting of THC, CBD, CBG, CBC, CBN, CBL THCV, CBDV and their non-decarboxylated form thereof; (b) a non-cannabinoid, non-terpene carrier, comprising less than 5% by weight cellulose; (c) a primary terpene, forming at least 40% by weight of the total terpene content, wherein the primary terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thujone, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

According to a related embodiment said THC and/or THCa is in a total concentration of at least 1% by weight, at least 2%, at least 3%, at least 4%, at least 5%, at least 6%, at least 8%, at least 10%, at least 12%, at least 14%, at least 16%, at least 18% or at least 20%, at least 30%, at least 40%, at least 50%, at least 600%, at least 70%, at least 80%, or at least 900% by weight.

According to a related embodiment said CBD and/or CBDa is in a total concentration of at least 1% by weight, at least 2%, at least 3%, at least 4%, at least 5%, at least 6%, at least 8%, at least 10%, at least 12%, at least 14%, at least 16%, at least 18% or at least 20%, at least 30%, at least 40%, at least 50%, at least 600%, at least 70%, at least 80%, or at least 900% by weight.

According to a related embodiment said CBG and/or CBGa is in a total concentration of at least 0.1% by weight, at least 0.5%, at least 1%, at least 2%, at least 3%, at least 4%, at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 900% by weight.

According to a related embodiment said CBC and/or CBCa is in a total concentration of at least 0.1% by weight, at least 0.5%, at least 1%, at least 2%, at least 3%, at least 4%, at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to a related embodiment said CBN and/or CBNa is in a total concentration of at least 0.1% by weight, at least 0.5%, at least 1%, at least 2%, at least 3%, at least 4%, at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to a related embodiment said CBL and/or CBLa is in a total concentration of at least 0.1% by weight, at least 0.5%, at least 1%, at least 2%, at least 3%, at least 4%, at least 5%, at least 6%, at least 8%, at least 10%, at least 12%, at least 14%, at least 16%, at least 18% or at least 20%, at least 30%, at least 40%, at least 50%, at least 600%, at least 70%, at least 800%, or at least 90% by weight.

According to a related embodiment said THCV and/or THCVa is in a total concentration of at least 0.1% by weight, at least 0.5%, at least 1%, at least 2%, at least 3%, at least 4%, at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to a related embodiment said CBDV and/or CBDVa is in a total concentration of at least 0.1% by weight, at least 0.5%, at least 1%, at least 2%, at least 3%, at least 4%, at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to a related embodiment said non-cannabinoid non-terpene carrier comprises less than 5% by weight cellulose, less than 4%, less than 3%, less than 2%, or less than 1%, or less than 0.5% by weight cellulose.

According to a related embodiment, said primary terpene forms at least 20% by weight of the total terpene content, at least 30%, at least 40%, at least 50%, at least 60%, at least 70%, at least 80% or at least 90% by weight of the total terpene content. According to a related embodiment, said primary terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thujone, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol and combinations thereof. According to an embodiment, said product comprises a liquid. According to an embodiment, said product is selected from the group consisting of tablets, gel capsules, medical patches, topicals, creams, varnish, sublingual oil, sprays, edibles, cannabis candies, cannabis drinks, cannabis baked products, suppositories, tampons, rectal candle, cigarettes, vaporizer liquid, nasal preparation, preparations containing micro and nano-emulsions, preparations containing micro and nano-particles and combinations thereof.

According to an embodiment, the product comprises less than 5% by weight glycol, less than 4%, less than 3%, less than 2%, or less than 1%, by weight glycol. As used herein, the term glycol refers to any glycol, including ethylene glycol, polyethylene glycol, propylene glycol and polypropylene glycol.

According to an embodiment, the product comprises water and water content is less than 30% by weight, less than 25%, less than 20%, less than 15%, less than 12%, less than 10%, or less than 8%. According to an embodiment, water content is at least 1% by weight, at least 2%, at least 3%, at least 4% or at least 5% by weight.

According to an embodiment, the product comprises chlorophyll. According to an embodiment, the product comprises at least 0.5% chlorophyll, at least 1, % at least 5%, at least 10%, at least 15%, at least 20% chlorophyll. According to an embodiment, the product comprises at least one flavonoid. According to an embodiment, the product comprises at least two, at least three, at least four, or at least five flavonoids. According to an embodiment, the product comprises at least one of isoflavones, coumestans, prenylflavonoids, lignans and saponins. According to an embodiment, the product comprises isoflavones. According to an embodiment, the product comprises coumestans. According to an embodiment, the product comprises prenylflavonoids. According to an embodiment, the product comprises lignans. According to an embodiment, the product comprises saponins. According to an embodiment, the product comprises at least one of biochanin A, formonenetin, genistein, daidzein, glycitein, prenylnaringenin, diosgenin, acetin and/or cimicifugoside. According to an embodiment, the product comprises biochanin A. According to an embodiment, the product comprises formonenetin. According to an embodiment, the product comprises genistein. According to an embodiment, the product comprises daidzein. According to an embodiment, the product comprises glycitein. According to an embodiment, the product comprises prenylnaringenin. According to an embodiment, the product comprises diosgenin. According to an embodiment, the product comprises acetin. According to an embodiment, the product comprises cimicifugoside.

According to an embodiment, the primary terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thujone, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

According to an embodiment, the product results in an increased therapeutic effect compared with that of a product comprising the same cannabinoids amounts and a smaller amount of the primary terpene, e.g. one half of that amount. According to various embodiment, the increased therapeutic effect has various forms, e.g. a shorter onset time, increased magnitude, extended duration, reduced dosages, reduced secondary adverse symptoms, reduced frequency of conditions and/or symptoms, reduced severity of conditions and/or symptoms, reduced consumption of other drugs and combinations thereof. According to an embodiment, the increased therapeutic effect comprises a shorter onset time, or differently put an earlier effect, which is important particularly in cases of sublingual and topical delivery and in cases where a rapid effect is desired, as in treating pain. According to an embodiment, the increased therapeutic effect comprises extended duration of the therapeutic effect, for example an extended time of pain relief. According to an embodiment, the increased therapeutic effect comprises increased magnitude of the therapeutic effect, enabling achieving a desired therapeutic effect on administering smaller doses of cannabinoids, saving thereby on cost. According to an embodiment, the increased therapeutic effect comprises using smaller doses of cannabinoids and still achieving at least the same beneficial result. According to an embodiment, the increased therapeutic effect comprises reduction of secondary adverse symptoms, e.g. adverse symptoms of the main illness, of ones of another illness and/or ones related to administered the product or other drugs. According to an embodiment, the increased therapeutic effect comprises reduced frequency of the conditions and/or symptoms. According to an embodiment, the increased therapeutic effect comprises reduced severity of the conditions and/or symptoms.

According to an embodiment, the primary terpene and the cannabinoids are present in specific amounts, and the onset time of the therapeutic effect, as measured by methods known in the art, is at least 20% shorter than that of a product comprising the same cannabinoids amounts and one half the amount of the primary terpene, at least 30%, at least 40%, at least 50%, at least 60%, or at least 70% shorter. According to an embodiment, the primary terpene, secondary terpenes and the cannabinoids are present in specific amounts, and the onset time of the therapeutic effect is at least 20% shorter than that of a product comprising the same cannabinoids amounts, same secondary terpene amounts and one half the amount of the primary terpene, at least 30%, at least 40%, at least 50%, at least 60%, or at least 70% short. Shorter onset time, or differently put an earlier effect, is important particularly in cases of sublingual and topical delivery and in cases where a rapid effect is desired, as in treating pain.

According to an embodiment, the primary terpene and the cannabinoids are present in specific amounts, and the onset time of the therapeutic effect is at least 20% longer than that of a product comprising the same cannabinoids amounts and one half the amount of the primary terpene, at least 30%, at least 40%, at least 50%, at least 60%, or at least 70% longer. According to an embodiment, the primary terpene, secondary terpenes and the cannabinoids are present in specific amounts, and the onset time of the therapeutic effect is at least 20% longer than that of a product comprising the same cannabinoids amounts, same secondary terpene amounts and one half the amount of the primary terpene, at least 30%, at least 40%, at least 50%, at least 60%, or at least 70% longer. According to an embodiment, products of delayed onset time are used in combination with shorter onset time to reach a sustained release effect.

According to an embodiment, the primary terpene and the cannabinoids are present in specific amounts, and the magnitude of the therapeutic effect, as measured by methods known in the art, is at least 20% greater compared with that of a product comprising the same cannabinoids amounts and one half the amount of the primary terpene, at least 30%, at least 40%, at least 50%, at least 60%, or at least 70% greater. According to an embodiment, the primary terpene, secondary terpenes and the cannabinoids are present in specific amounts, and the magnitude of the therapeutic effect is at least 20% greater than that of a product comprising the same cannabinoids amounts, same secondary terpene amounts and one half the amount of the primary terpene, at least 30%, at least 40%, at least 50%, at least 60%, or at least 70% greater. Without wishing to be limited by any particular theory, such increased magnitude of the therapeutic effect may indicate increased bioavailability. Such increased magnitude enables achieving a desired therapeutic effect on administering smaller doses of cannabinoids, saving thereby on cost.

According to an embodiment, the primary terpene and the cannabinoids are present in specific amounts, and the duration of the therapeutic effect, as measured by methods known in the art, is at least 20% longer compared with that of a product comprising the same cannabinoids amounts and one half the amount of the primary terpene, at least 30%, at least 40%, at least 50%, at least 60%, at least 70%, at least 80%, at least 90% or at least 100% longer. According to an embodiment, the primary terpene, secondary terpenes and the cannabinoids are present in specific amounts, and the duration of the therapeutic effect is at least 20% longer than that of a product comprising the same cannabinoids amounts, same secondary terpene amounts and one half the amount of the primary terpene, at least 30%, at least 40%, at least 50%, at least 60%, at least 70%, at least 80%, at least 90% or at least 100% longer.

According to an embodiment, the primary terpene and the cannabinoids are present in specific amounts, and the frequency of the conditions and/or symptoms, as measured by methods known in the art, is at least 20% smaller compared with that of a product comprising the same cannabinoids amounts and one half the amount of the primary terpene, at least 30%, at least 40%, at least 50% at least 60%, at least 70%, at least 80%, or at least 90% smaller. According to an embodiment, the primary terpene, secondary terpenes and the cannabinoids are present in specific amounts, and the frequency of the conditions and/or symptoms, as measured by methods known in the art, is at least 20% smaller than that of a product comprising the same cannabinoids amounts, same secondary terpene amounts and one half the amount of the primary terpene, at least 30%, at least 40%, at least 50%, at least 60%, at least 70%, at least 80%, at least 90% or at least 100% longer.

According to an embodiment, the primary terpene and the cannabinoids are present in specific amounts, and the severity of the conditions and/or symptoms, as measured by methods known in the art, is at least 20% smaller compared with that of a product comprising the same cannabinoids amounts and one half the amount of the primary terpene, at least 30%, at least 40%, at least 50% at least 60%, at least 70%, at least 80%, or at least 90% smaller. According to an embodiment, the primary terpene, secondary terpenes and the cannabinoids are present in specific amounts, and the severity of the conditions and/or symptoms, as measured by methods known in the art, is at least 20% smaller than that of a product comprising the same cannabinoids amounts, same secondary terpene amounts and one half the amount of the primary terpene, at least 30%, at least 40%, at least 50%, at least 60%, at least 70%, at least 80%, at least 90% or at least 100% smaller.

According to an embodiment, the primary terpene and the cannabinoids are present in specific amounts, and the consumption of other drugs is reduced by at least 20% compared with that of a product comprising the same cannabinoids amounts and one half the amount of said primary terpene, reduced by at least 30%, by at least 40%, by at least 50%, by at least 60%, by at least 70%, by at least 80%, or by at least 900%. According to an embodiment, the primary terpene, secondary terpenes and the cannabinoids are present in specific amounts, and the consumption of other drugs, as measured by methods known in the art, is reduced by at least 20% compared with a product comprising the same cannabinoids amounts, same secondary terpene amounts and one half the amount of the primary terpene, reduced by at least 30%, by at least 40%, by at least 50%, by at least 60%, by at least 70%, by at least 80%, or by at least 900%.

According to an embodiment, the therapeutic effect is generated while administering the product in a vaporizer. According to an embodiment, the therapeutic effect is generated while administering the product by inhaling. According to an embodiment, the therapeutic effect is generated while administering the product by smoking. According to an embodiment, the therapeutic effect is generated while administering the product sublingually. According to an embodiment, the therapeutic effect is generated while applying the product, topically. According to an embodiment, the therapeutic effect is generated while administering the product in suppositories, including tampons and/or rectal candles. According to an embodiment, the therapeutic effect is generated while administering the product in a spray. According to an embodiment, the therapeutic effect is generated while administering the product as an edible, as a candy, as baked good, as a drink. According to an embodiment, the therapeutic effect is generated while administering the product as vaporizer liquid nasal preparation. According to an embodiment, the therapeutic effect is generated while administering the product as micro and nano-emulsions. According to an embodiment, the therapeutic effect is generated while administering the product as micro and nano-emulsions.

According to an embodiment said therapeutic effect treats osteoporosis and said product comprises at least one of CBD, THC, CBC, CBG, CBN, CBL, THCV and CBDV. According to an embodiment said therapeutic effect treats osteoporosis and said product comprises CBD and optionally THC at CBD to THC weight/weight ratio greater than 1, greater than 2, greater than 3, greater than 4 or greater than 5. According to an embodiment said therapeutic effect treats osteoporosis and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats osteoporosis and said the primary terpene is selected from the group consisting of caryophyllene, caryophyllene oxide, carene, eucalyptol, sabinene, pinene, limonene, borneol, thymol, camphor and combinations thereof. According to an embodiment, the product comprises at least 2 of these terpenes, at least 3, at least 4, or at least 5 of the terpenes. According to an embodiment, the primary terpene is selected from the group consisting of caryophyllene, carene, borneol and combinations thereof. According to various embodiments said primary terpene comprises caryophyllene oxide, eucalyptol, sabinene, pinene, limonene, thymol and/or camphor. According to an embodiment, said therapeutic effect treats osteoporosis and the product further comprises a compound selected from the group consisting of biochanin A, formonenetin, genistein, daidzein, glycitein, prenylnaringenin, diosgenin, acetin, cimicifugoside and combinations thereof.

According to an embodiment said therapeutic effect treats sleep disorder and/or insomnia and said product comprises at least one of CBD, THC, CBC, CBG, CBN, CBL, THCV and CBDV. According to an embodiment said therapeutic effect treats sleep disorder and/or insomnia and said product comprises CBD and optionally THC at CBD to THC weight/weight ratio greater than 1, greater than 2, greater than 3, greater than 4 or greater than 5. According to an embodiment said therapeutic effect treats sleep disorder and/or insomnia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats sleep disorder and/or insomnia and said the primary terpene is selected from the group consisting of linalool, caryophyllene, myrcene, terpineol, terpinene, citronellol, eucalyptol, nerolidol, sabinene, pinene, limonene, borneol, fenchol, bisabolol humulene, phytol and combinations thereof. According to an embodiment, the product comprises at least 2 of these terpenes, at least 3, at least 4, or at least 5 of the terpenes. According to an embodiment, the primary terpene is selected from the group consisting of linalool, caryophyllene, myrcene and combinations thereof. According to an embodiment, the primary terpene is selected from the group consisting of linalool, nerolidol, myrcene and combinations thereof. According to various embodiments said primary terpene comprises terpineol, terpinene, citronellol, eucalyptol, nerolidol, sabinene, pinene, limonene, borneol, fenchol, bisabolol, phytol and/or humulene. According to an embodiment, said therapeutic effect treats sleep disorder and/or insomnia and the product further comprises a compound selected from the group consisting of linalyl acetate, chamazulene, biochanin A, formonenetin, genistein, daidzein, glycitein, prenylnaringenin, diosgenin, acetin, cimicifugoside and combinations thereof. According to an embodiment, the product includes caryophyllene, and caryophyllene forms less than 5% by weight of the total terpene content.

According to an embodiment said therapeutic effect treats anxiety, irritability, nervousness, restless, stress, depression, mood swing and/or affect disorder and said product comprises at least one of CBD, THC, CBC, CBG, CBN, CBL, THCV and CBDV.

According to an embodiment said therapeutic effect treats anxiety, irritability, nervousness, restless, stress, depression, mood swing and/or affect disorder and said product comprises CBD and optionally THC at CBD to THC weight/weight ratio greater than 1, greater than 2, greater than 3, greater than 4 or greater than 5.

According to an embodiment said therapeutic effect treats anxiety, irritability, nervousness, restless, stress, depression, mood swing and/or affect disorder and said product comprises THC and optionally CBD at THC to CBD weight/weight ratio greater than 0.9, greater than 2, greater than 3, greater than 4 or greater than 5.

According to various embodiments, said product comprises CBD, comprises THC, comprises CBC, comprises CBG, comprises CBN, comprises CBL, comprises THCV and/or comprises CBDV.

According to an embodiment said therapeutic effect treats anxiety, irritability, nervousness, restless, stress, depression, mood swing and/or affect disorder and said the primary terpene is selected from the group consisting of limonene, linalool, terpineol, citronellol, eucalyptol, caryophyllene, geraniol, menthol, iso-menthone, germacrene, pinene, citronellol, cardinol, neral, geranial, myrcene, terpinene, menth-2-en-1-ol, humulene, bisabolol, borneol, fenchol, nerolidol and combinations thereof.

According to an embodiment, the product comprises at least 2 of these terpenes, at least 3, at least 4, or at least 5 of the terpenes. According to an embodiment, the primary terpene is selected from the group consisting of limonene, linalool, eucalyptol and combinations thereof. According to an embodiment, the primary terpene is selected from the group consisting of limonene, linalool, caryophyllene, bisabolol and combinations thereof. According to an embodiment, the primary terpene is selected from the group consisting of limonene, linalool, pinene, humulene and combinations thereof. According to various embodiments said primary terpene comprises terpineol, citronellol, caryophyllene, geraniol, menthol, iso-menthone, germacrene, pinene, citronellol, cardinol, neral, geranial, myrcene, terpinene, menth-2-en-1-ol, humulene, bisabolol, borneol, nerolidol or fenchol. According to an embodiment, said therapeutic effect treats anxiety, irritability, nervousness, restless, stress, depression, mood swing and/or affect disorder and the product further comprises a compound selected from the group consisting of linalyl acetate, geranyl acetate, menthol, piperine, eugenol, citronellyl formate, jasmone, benzy acetate, benzyl alcohol, biochanin A, formonenetin, genistein, daidzein, glycitein, prenylnaringenin, diosgenin, acetin, cimicifugoside and combinations thereof. According to an embodiment, the product includes pinene, terpineol, bisabolol, caryophyllene, myrcene and/or limonene, and pinene, terpineol, bisabolol, caryophyllene, myrcene and/or limonene forms less than 5% by weight of the total terpene content.

According to an embodiment said therapeutic effect treats difficulty in concentrating, brain fog and/or memory loss and said product comprises at least one of CBD, THC, CBC, CBG, CBN, CBL, THCV and CBDV. According to an embodiment said therapeutic effect treats difficulty in concentrating, brain fog and/or memory loss and said product comprises CBD and optionally THC at CBD to THC weight/weight ratio greater than 1, greater than 2, greater than 3, greater than 4 or greater than 5. According to an embodiment said therapeutic effect treats difficulty in concentrating, brain fog and/or memory loss and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats difficulty in concentrating, brain fog and/or memory loss and said the primary terpene is selected from the group consisting of eucalyptol, pinene, carene, limonene, pulegone, terpineol, cymene, linalool, caryophyllene and combinations thereof. According to an embodiment, the product comprises at least 2 of these terpenes, at least 3, at least 4, or at least 5 of the terpenes. According to an embodiment, the primary terpene is selected from the group consisting of eucalyptol, pinene, limonene and combinations thereof. According to various embodiments said primary terpene comprises carene, pulegone, terpineol, linalool, caryophyllene and/or cymene According to an embodiment, said therapeutic effect treats difficulty in concentrating, brain fog and/or memory loss and the product further comprises a compound selected from the group consisting of biochanin A, formonenetin, genistein, daidzein, glycitein, prenylnaringenin, diosgenin, acetin, cimicifugoside and combinations thereof.

According to an embodiment said therapeutic effect treats general discomfort and said product comprises at least one of CBD, THC, CBC, CBG, CBN, CBL, THCV and CBDV. According to an embodiment said therapeutic effect treats general discomfort and said product comprises CBD and optionally THC at CBD to THC weight/weight ratio greater than 1, greater than 2, greater than 3, greater than 4 or greater than 5. According to an embodiment said therapeutic effect treats general discomfort and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV.

According to an embodiment said therapeutic effect treats general discomfort and said the primary terpene is selected from the group consisting of limonene, linalool humulene, terpinene, borneol, caryophyllene and combinations thereof. According to an embodiment, the product comprises at least 2 of these terpenes, at least 3, at least 4, or at least 5 of the terpenes. According to an embodiment, the primary terpene is selected from the group consisting of linalool, humulene, terpinene and combinations thereof. According to various embodiments said primary terpene comprises limonene, borneol and/or caryophyllene. According to an embodiment, the product includes myrcene, pinene and/or bisabolol, and myrcene, pinene and/or bisabolol forms less than 5% by weight of the total terpene content.

According to an embodiment said therapeutic effect treats muscle tension and/or myalgia and said product comprises at least one of CBD, THC, CBC, CBG, CBN, CBL, THCV and CBDV. According to an embodiment said therapeutic effect treats muscle tension and/or myalgia and said product comprises CBD and optionally THC at CBD to THC weight/weight ratio greater than 1, greater than 2, greater than 3, greater than 4 or greater than 5. According to an embodiment said therapeutic effect treats muscle tension and/or myalgia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats muscle tension and/or myalgia and said the primary terpene is selected from the group consisting of myrcene, pinene, nerolidol, linalool, humulene, caryophyllene, limonene, bisabolol and combinations thereof. According to an embodiment, the product comprises at least 2 of these terpenes, at least 3, at least 4, or at least 5 of the terpenes. According to an embodiment, the primary terpene is selected from the group consisting of myrcene, pinene, nerolidol and combinations thereof. According to an embodiment, the primary terpene is selected from the group consisting of myrcene, pinene, nerolidol, linalool, humulene and combinations thereof. According to various embodiments said primary terpene comprises linalool, humulene, caryophyllene, limonene and/or bisabolol.

According to an embodiment said therapeutic effect treats fatigue and/or intermittent dizziness and said product comprises at least one of CBD, THC, CBC, CBG, CBN, CBL, THCV and CBDV. According to an embodiment said therapeutic effect treats fatigue and/or intermittent dizziness and said product comprises CBD and optionally THC at CBD to THC weight/weight ratio greater than 1, greater than 2, greater than 3, greater than 4 or greater than 5. According to an embodiment said therapeutic effect treats fatigue and/or intermittent dizziness and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats fatigue and/or intermittent dizziness and said the primary terpene is selected from the group consisting of pinene, limonene, terpinene, eucalyptol, myrcene, terpineol, linalool, caryophyllene and combinations thereof. According to an embodiment, the product comprises at least 2 of these terpenes, at least 3, at least 4, or at least 5 of the terpenes. According to an embodiment, the primary terpene is selected from the group consisting of pinene, limonene, eucalyptol and combinations thereof. According to various embodiments said primary terpene comprises terpinene, myrcene and/or caryophyllene.

According to an embodiment said therapeutic effect treats menopausal skin, itchy skin, age-related wrinkles and/or stretch marks and said product comprises at least one of CBD, THC, CBC, CBG, CBN, CBL, THCV and CBDV. According to an embodiment said therapeutic effect treats menopausal skin, itchy skin, age-related wrinkles and/or stretch marks and said product comprises CBD and optionally THC at CBD to THC weight/weight ratio greater than 1, greater than 2, greater than 3, greater than 4 or greater than 5. According to an embodiment said therapeutic effect treats menopausal skin, itchy skin, age-related wrinkles and/or stretch marks and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats menopausal skin, itchy skin, age-related wrinkles and/or stretch marks and said the primary terpene is selected from the group consisting of linalool limonene, nerolidol, pinene terpineol, myrcene, geraniol, nerol, citronellol, farnesol, carotol and combinations thereof. According to an embodiment, the product comprises at least 2 of these terpenes, at least 3, at least 4, or at least 5 of the terpenes. According to an embodiment, the primary terpene is selected from the group consisting of linalool, terpineol, geraniol and combinations thereof. According to various embodiments said primary terpene comprises limonene, nerolidol, pinene myrcene, nerol, citronellol, farnesol and/or carotol. According to an embodiment, said therapeutic effect treats menopausal skin, itchy skin, age-related wrinkles and/or stretch marks and the product further comprises a compound selected from the group consisting of linalyl acetate, citronellyl formate, eugenol, benzy acetate, benzyl alcohol, biochanin A, formonenetin, genistein, daidzein, glycitein, prenylnaringenin, diosgenin, acetin, cimicifugoside and combinations thereof.

According to an embodiment said therapeutic effect treats headache and/or migraine and said product comprises at least one of CBD, THC, CBC, CBG, CBN, CBL, THCV and CBDV. According to an embodiment said therapeutic effect treats headache and/or migraine and said product comprises CBD and optionally THC at CBD to THC weight/weight ratio greater than 1, greater than 2, greater than 3, greater than 4 or greater than 5. According to an embodiment said therapeutic effect treats headache and/or migraine and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats headache and/or migraine and said the primary terpene is selected from the group consisting of myrcene, linalool, caryophyllene, eucalyptol, limonene, nerolidol, fenchol, humulene, guaiol, borneol, pinene, bisabolol, terpinene, terpineol and combinations thereof. According to an embodiment, the product comprises at least 2 of these terpenes, at least 3, at least 4, or at least 5 of the terpenes. According to an embodiment, the primary terpene is selected from the group consisting of myrcene, linalool, caryophyllene and combinations thereof. According to various embodiments said primary terpene comprises eucalyptol, limonene, nerolidol, fenchol, humulene, guaiol, linalool, limonene, borneol, pinene and/or bisabolol. According to an embodiment, the product includes bisabolol, terpinene, humulene and/or linalool wherein bisabolol, terpinene, humulene and/or linalool forms less than 5% by weight of the total terpene content.

According to an embodiment said therapeutic effect treats weight gain and said product comprises at least one of CBD, THC, CBC, CBG, CBN, CBL, THCV and CBDV. According to an embodiment said therapeutic effect treats weight gain and said product comprises CBD and optionally THC at CBD to THC weight/weight ratio greater than 1, greater than 2, greater than 3, greater than 4 or greater than 5. According to an embodiment said therapeutic effect treats weight gain and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats weight gain and said the primary terpene is selected from the group consisting of eucalyptol, caryophyllene, limonene, humulene, phytol and combinations thereof. According to an embodiment, the product comprises at least 2 of these terpenes, at least 3, at least 4, or at least 5 of the terpenes. According to an embodiment, the primary terpene is selected from the group consisting of eucalyptol, limonene, humulene and combinations thereof. According to various embodiments said primary terpene comprises caryophyllene and/or phytol.

According to an embodiment said therapeutic effect treats digestive problems, intestinal disorders, abdominal pain (colic), nausea, constipation and/or diarrhea and said product comprises at least one of CBD, THC, CBC, CBG, CBN, CBL, THCV and CBDV. According to an embodiment said therapeutic effect treats digestive problems, intestinal disorders, abdominal pain (colic), nausea, constipation and/or diarrhea and said product comprises CBD and optionally THC at CBD to THC weight/weight ratio greater than 1, greater than 2, greater than 3, greater than 4 or greater than 5. According to an embodiment said therapeutic effect treats digestive problems, intestinal disorders, abdominal pain (colic), nausea, constipation and/or diarrhea and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats digestive problems, intestinal disorders, abdominal pain (colic), nausea, constipation and/or diarrhea and said the primary terpene is selected from the group consisting of limonene, cymene, terpinene, caryophyllene, citronellol, sabinene, myrcene, linalool, germacrene, geraniol, pinene, terpineol and combinations thereof. According to an embodiment, the product comprises at least 2 of these terpenes, at least 3, at least 4, or at least 5 of the terpenes. According to an embodiment, the primary terpene is selected from the group consisting of limonene, sabinene, myrcene and combinations thereof. According to various embodiments said primary terpene comprises cymene, terpinene, caryophyllene, citronellol, linalool, germacrene, geraniol, terpineol and/or pinene. According to an embodiment, said therapeutic effect treats digestive problems, intestinal disorders, abdominal pain (colic), nausea, constipation and/or diarrhea and the product further comprises piperine. According to an embodiment, the product includes pinene, bisabolol, limonene and/or terpineol, wherein pinene, bisabolol, limonene and/or terpineol forms less than 5% by weight of the total terpene content

According to an embodiment said therapeutic effect treats breast pain and/or Mastalgia and said product comprises at least one of CBD, THC, CBC, CBG, CBN, CBL, THCV and CBDV. According to an embodiment said therapeutic effect treats breast pain and/or Mastalgia and said product comprises CBD and optionally THC at CBD to THC weight/weight ratio greater than 1, greater than 2, greater than 3, greater than 4 or greater than 5. According to an embodiment said therapeutic effect treats breast pain and/or Mastalgia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats pain and/or Mastalgia and said the primary terpene is selected from the group consisting of pinene, limonene, caryophyllene, myrcene, linalool, geraniol, eucalyptol, terpineol, sabinene and combinations thereof. According to an embodiment, the product comprises at least 2 of these terpenes, at least 3, at least 4, or at least 5 of the terpenes. According to an embodiment, the primary terpene is selected from the group consisting of pinene, linalool, eucalyptol and combinations thereof. According to various embodiments said primary terpene comprises limonene, caryophyllene, myrcene, geraniol, terpineol and/or sabinene. According to an embodiment, said therapeutic effect treats pain and/or Mastalgia and the product further comprises a compound selected from the group consisting of biochanin A, formonenetin, genistein, daidzein, glycitein, prenylnaringenin, diosgenin, acetin, cimicifugoside and combinations thereof.

According to an embodiment said therapeutic effect treats dysmenorrhea, pelvic pain, spasms, cramps, labor contraction and/or endometriosis and said product comprises at least one of CBD, THC, CBC, CBG, CBN, CBL, THCV and CBDV. According to an embodiment said therapeutic effect treats dysmenorrhea, pelvic pain, spasms, cramps, labor contraction and/or endometriosis and said product comprises CBD and optionally THC at CBD to THC weight/weight ratio greater than 1, greater than 2, greater than 3, greater than 4 or greater than 5. According to an embodiment said therapeutic effect treats dysmenorrhea, pelvic pain, spasms, cramps, labor contraction and/or endometriosis and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats dysmenorrhea, pelvic pain, spasms, cramps, labor contraction and/or endometriosis and said the primary terpene is selected from the group consisting of myrcene, linalool, caryophyllene, eucalyptol, nerolidol, limonene, nerolidol, sabinene, humulene, terpineol, citronellol, geraniol, pinene, limonene, camphene, terpinene, borneol, camphor, menthone, iso-menthone, carvacrol, thymol, carene, nerol, farnesol, menth-2-en-1-ol and combinations thereof. According to an embodiment, the product comprises at least 2 of these terpenes, at least 3, at least 4, or at least 5 of the terpenes. According to an embodiment, the primary terpene is selected from the group consisting of linalool, eucalyptol, nerolidol and combinations thereof. According to an embodiment, the primary terpene is selected from the group consisting of linalool, myrcene, nerolidol and combinations thereof. According to various embodiments said primary terpene comprises myrcene, caryophyllene, limonene, nerolidol, sabinene, humulene, terpineol, citronellol, geraniol, pinene, limonene, camphene, terpinene, borneol, camphor, menthone, iso-menthone, carvacrol, thymol, carene, nerol, farnesol and/or menth-2-en-ol. According to an embodiment, said therapeutic effect treats dysmenorrhea, pelvic pain, spasms, cramps, labor contraction and/or endometriosis and the product further comprises a compound selected from the group consisting of linalyl acetate, geranyl acetate, eugenol, methylis-oeugenol, menthol, eugenyl acetate, piperine, chamazulene, citronllyl formate, benzyl acetate, benzyl alcohol, biochanin A, formonenetin, genistein, daidzein, glycitein, prenylnaringenin, diosgenin, acetin, cimicifugoside and combinations thereof.

According to an embodiment said therapeutic effect treats arthralgia and/or arthritis and said product comprises at least one of CBD, THC, CBC, CBG, CBN, CBL, THCV and CBDV. According to an embodiment said therapeutic effect treats arthralgia and/or arthritis and said product comprises CBD and optionally THC at CBD to THC weight/weight ratio greater than 1, greater than 2, greater than 3, greater than 4 or greater than 5. According to an embodiment said therapeutic effect treats arthralgia and/or arthritis and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats arthralgia and/or arthritis and said the primary terpene is selected from the group consisting of myrcene, linalool, guaiol, caryophyllene, eucalyptol, pinene, nerolidol, sabinene, limonene, terpinene, menth-2-en-1-ol, geraniol, guaiol, humulene, bisabolol, borneol, fenchol, terpineol and combinations thereof. According to an embodiment, the product comprises at least 2 of these terpenes, at least 3, at least 4, or at least 5 of the terpenes. According to an embodiment, the primary terpene is selected from the group consisting of myrcene, pinene, sabinene and combinations thereof. According to an embodiment, the primary terpene is selected from the group consisting of terpineol, linalool, sabinene and combinations thereof. According to various embodiments said primary terpene comprises linalool, guaiol, caryophyllene, eucalyptol, nerolidol, limonene, terpinene, menth-2-en-1-ol, geraniol, guaiol, humulene, bisabolol, borneol, fenchol and/or terpineol. According to an embodiment, said therapeutic effect treats arthralgia and/or arthritis and the product further comprises a compound selected from the group consisting of linalyl acetate, geranyl acetate, eugenol, methylis-oeugenol, menthol, eugenyl acetate, piperine and combinations thereof. According to an embodiment, the product optionally includes humulene, pinene, bisabolol, borneol, fenchol, guaiol, linalool, ocimene, terpineol, terpinene and/or caryophyllene, wherein humulene, pinene, bisabolol, borneol, fenchol, guaiol, linalool, ocimene, terpineol, terpinene and/or caryophyllene forms less than 5% by weight of the total terpene content.

According to an embodiment said therapeutic effect treats hot flashes, facial flushing, night sweat, irritability, palpitation and/or tachycardia and said product comprises at least one of CBD, THC, CBC, CBG, CBN, CBL, THCV and CBDV. According to an embodiment said therapeutic effect treats hot flashes, facial flushing, night sweat, irritability, palpitation and/or tachycardia and said product comprises CBD and optionally THC at CBD to THC weight/weight ratio greater than 1, greater than 2, greater than 3, greater than 4 or greater than 5. According to an embodiment said therapeutic effect treats hot flashes, facial flushing, night sweat, irritability, palpitation and/or tachycardia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats hot flashes, facial flushing, night sweat, irritability, palpitation and/or tachycardia and said the primary terpene is selected from the group consisting of caryophyllene, myrcene, humulene, linalool, terpineol, geraniol, pinene, carene, citronellol, limonene, eucalyptol, sabinene, myrtenol, terpinene, champhene, borneol and combinations thereof. According to an embodiment, the product comprises at least 2 of these terpenes, at least 3, at least 4, or at least 5 of the terpenes. According to an embodiment, the primary terpene is selected from the group consisting of linalool, pinene, eucalyptol and combinations thereof. According to an embodiment, the primary terpene is selected from the group consisting of linalool, geraniol, limonene, eucalyptol and combinations thereof. According to various embodiments said primary terpene comprises caryophyllene, myrcene, humulene, terpineol, geraniol, pinene, carene, citronellol, limonene, sabinene, myrtenol, champhene, borneol and/or terpinene. According to an embodiment, said therapeutic effect treats hot flashes, facial flushing, night sweat, irritability, palpitation and/or tachycardia and the product further comprises a compound selected from the group consisting of linalyl acetate, geranyl acetate, citronellyl formate, biochanin A, formonenetin, genistein, daidzein, glycitein, prenylnaringenin, diosgenin, acetin, cimicifugoside and combinations thereof.

According to an embodiment said therapeutic effect treats inflammation, oxidative stress and/or paresthesia and said product comprises at least one of CBD, THC, CBC, CBG, CBN, CBL, THCV and CBDV. According to an embodiment said therapeutic effect treats inflammation, oxidative stress and/or paresthesia and said product comprises CBD and optionally THC at CBD to THC weight/weight ratio greater than 1, greater than 2, greater than 3, greater than 4 or greater than 5. According to an embodiment said therapeutic effect treats inflammation, oxidative stress and/or paresthesia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats inflammation, oxidative stress and/or paresthesia and said the primary terpene is selected from the group consisting of caryophyllene, pinene, myrcene, limonene, humulene, citronellol, eucalyptol, linalool, beta amyrin, cycloartenol, terpinene, terpineol, geraniol, carvacrol, thymol, cardinol, terpinene, menth-2-en-1-ol, carotol, humulene, nerolidol and combinations thereof. According to an embodiment, the product comprises at least 2 of these terpenes, at least 3, at least 4, or at least 5 of the terpenes. According to an embodiment, the primary terpene is selected from the group consisting of caryophyllene, pinene, eucalyptol and combinations thereof. According to various embodiments said primary terpene comprises myrcene, limonene, humulene, citronellol, linalool, beta amyrin, cycloartenol, terpinene, terpineol, geraniol, carvacrol, thymol, cardinol, terpinene, menth-2-en-1-ol, carotol, humulene and/or nerolidol. According to an embodiment, said therapeutic effect treats inflammation, oxidative stress and/or paresthesia and the product further comprises a compound selected from the group consisting of linalyl acetate, geranyl acetate, eugenol, eugenyl acetate, piperine and combinations thereof.

According to an embodiment said therapeutic effect treats cardiovascular disease, palpitation and tachycardia and said product comprises at least one of CBD, THC, CBC, CBG, CBN, CBL, THCV and CBDV. According to an embodiment said therapeutic effect treats cardiovascular disease, palpitation and tachycardia and said product comprises CBD and optionally THC at CBD to THC weight/weight ratio greater than 1, greater than 2, greater than 3, greater than 4 or greater than 5. According to an embodiment said therapeutic effect treats cardiovascular disease, palpitation and tachycardia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats cardiovascular disease, palpitation and tachycardia and said the primary terpene is selected from the group consisting of linalool, camphene, limonene, pinene, borneol and combinations thereof. According to an embodiment, the product comprises at least 2 of these terpenes, at least 3, at least 4, or all 5 of the terpenes. According to an embodiment, the primary terpene is selected from the group consisting of linalool, limonene, pinene and combinations thereof. According to various embodiments said primary terpene comprises camphene and/or borneol.

According to an embodiment said therapeutic effect treats urine incontinence and/or lower urinary tract disorders and said product comprises at least one of CBD, THC, CBC, CBG, CBN, CBL, THCV and CBDV. According to an embodiment said therapeutic effect treats urine incontinence and/or lower urinary tract disorders and said product comprises CBD and optionally THC at CBD to THC weight/weight ratio greater than 1, greater than 2, greater than 3, greater than 4 or greater than 5. According to an embodiment said therapeutic effect treats urine incontinence and/or lower urinary tract disorders and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats urine incontinence and/or lower urinary tract disorders and said the primary terpene is selected from the group consisting of linalool, terpineol, pinene, carene, geraniol, thujone, camphor, viridiflorol, caryophyllene, germacrene, cirtonellol, eucalyptol, myrtenol, sanatalol, sabinene, limonene and combinations thereof. According to an embodiment, the product comprises at least 2 of these terpenes, at least 3, at least 4, or at least 5 of the terpenes. According to an embodiment, the primary terpene is selected from the group consisting of linalool, geraniol, pinene and combinations thereof. According to various embodiments said primary terpene comprises terpineol, carene, thujone, camphor, viridiflorol, caryophyllene, germacrene, cirtonellol, eucalyptol, myrtenol, sanatalol, sabinene and/or limonene According to an embodiment, said therapeutic effect treats urine incontinence and/or lower urinary tract disorders and the product further comprises a compound selected from the group consisting of linalyl acetate, geranyl acetate, citronellyl formate and combinations thereof.

According to an embodiment said therapeutic effect treats vaginal dryness and said product comprises at least one of CBD, THC, CBC, CBG, CBN, CBL, THCV and CBDV. According to an embodiment said therapeutic effect treats vaginal dryness and said product comprises CBD and optionally THC at CBD to THC weight/weight ratio greater than 1, greater than 2, greater than 3, greater than 4 or greater than 5. According to an embodiment said therapeutic effect treats vaginal dryness and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV.

According to an embodiment said therapeutic effect treats vaginal dryness and said the primary terpene is selected from the group consisting of myrcene, humulene, caryophyllene, linalool, terpineol, geraniol, pinene, germacrene, cirtonellol, limonene, myrcene, terpinene, menth-2-en-1-ol, eucalyptol, myrtenol and combinations thereof. According to an embodiment, the product comprises at least 2 of these terpenes, at least 3, at least 4, or at least 5 of the terpenes. According to an embodiment, the primary terpene is selected from the group consisting of linalool, geraniol, pinene and combinations thereof. According to various embodiments said primary terpene myrcene, humulene, caryophyllene, terpineol, germacrene, cirtonellol, limonene, myrcene, terpinene, menth-2-en-1-ol, eucalyptol and/or myrtenol. According to an embodiment, said therapeutic effect treats vaginal dryness and the product further comprises a compound selected from the group consisting of linalyl acetate, geranyl acetate, citronellyl formate, biochanin A, formonenetin, genistein, daidzein, glycitein, prenylnaringenin, diosgenin, acetin, cimicifugoside and combinations thereof.

According to an embodiment said therapeutic effect treats female urogenital and/or reproductive system infection and said product comprises at least one of CBD, THC, CBC, CBG, CBN, CBL, THCV and CBDV. According to an embodiment said therapeutic effect treats female urogenital and/or reproductive system infection and said product comprises CBD and optionally THC at CBD to THC weight/weight ratio greater than 1, greater than 2, greater than 3, greater than 4 or greater than 5. According to an embodiment said therapeutic effect treats female urogenital and/or reproductive system infection and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats female urogenital and/or reproductive system infection and said the primary terpene is selected from the group consisting of pinene, limonene, cymene, caryophyllene oxide, ocimene, nerolidol, eucalyptol, terpinene, terpineol, camphor, linalool, sabinene, thujone, camphor, viridiflorol, menthol, iso-menthone, caryophyllene, germacrene, geraniol, nerol, neral, geranial, citronellol, carvacol, thymol, carene, myrtenol, carene, myrcene, menth-2-en-1-ol and combinations thereof. According to an embodiment, the product comprises at least 2 of these terpenes, at least 3, at least 4, or at least 5 of the terpenes. According to an embodiment, the primary terpene is selected from the group consisting of caryophyllene oxide, pinene, limonene, geraniol and combinations thereof. According to an embodiment, the primary terpene is selected from the group consisting of caryophyllene oxide, pinene, limonene and combinations thereof. According to various embodiments said primary terpene comprises cymene, ocimene, nerolidol, eucalyptol, terpinene, terpineol, camphor, linalool, sabinene, thujone, camphor, viridiflorol, menthol, iso-menthone, caryophyllene, germacrene, geraniol, nerol, neral, geranial, citronellol, carvacol, thymol, carene, myrtenol, carene, myrcene and/or menth-2-en-1-ol. According to an embodiment, said therapeutic effect treats female urogenital and/or reproductive system infection and the product further comprises a compound selected from the group consisting of linalyl acetate, geranyl acetate, eugenol, eugenyl acetate, piperine, menthol, citronellyl formate and combinations thereof. According to an embodiment the therapeutic effect treats human papillomavirus (HPV).

According to an embodiment said therapeutic effect treats fibroid, myomas and/or cysts in the female genital system and said product comprises at least one of CBD, THC, CBC, CBG, CBN, CBL, THCV and CBDV. According to an embodiment said therapeutic effect treats fibroid, myomas and/or cysts in the female genital system and said product comprises CBD and optionally THC at CBD to THC weight/weight ratio greater than 1, greater than 2, greater than 3, greater than 4 or greater than 5. According to an embodiment said therapeutic effect treats fibroid, myomas and/or cysts in the female genital system and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats fibroid, myomas and/or cysts in the female genital system and said the primary terpene is selected from the group consisting of linalool, terpineol, limonene, pinene, caryophyllene, caryophyllene oxide, terpinene, citronellol and combinations thereof. According to an embodiment, the product comprises at least 2 of these terpenes, at least 3, at least 4, or at least 5 of the terpenes. According to an embodiment, the primary terpene is selected from the group consisting of limonene, pinene, terpinene and combinations thereof. According to various embodiments said primary terpene comprises linalool, terpineol, caryophyllene, caryophyllene oxide and/or citronellol.

According to an embodiment said therapeutic effect treats cystitis, urethritis and/or pyelonephritis and said product comprises at least one of CBD, THC, CBC, CBG, CBN, CBL, THCV and CBDV. According to an embodiment said therapeutic effect treats cystitis, urethritis and/or pyelonephritis and said product comprises CBD and optionally THC at CBD to THC weight/weight ratio greater than 1, greater than 2, greater than 3, greater than 4 or greater than 5. According to an embodiment said therapeutic effect treats cystitis, urethritis and/or pyelonephritis and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats cystitis, urethritis and/or pyelonephritis and said the primary terpene is selected from the group consisting of thujone, pinene, limonene, cymene, caryophyllene, caryophyllene oxide, eucalyptol, terpineol, sabinene, linalool, cardinol and combinations thereof. According to an embodiment, the product comprises at least 2 of these terpenes, at least 3, at least 4, or at least 5 of the terpenes. According to an embodiment, the primary terpene is selected from the group consisting of limonene, eucalyptol, linalool and combinations thereof. According to an embodiment, the primary terpene is selected from the group consisting of limonene, pinene, linalool and combinations thereof. According to various embodiments said primary terpene comprises thujone, pinene, cymene, caryophyllene, caryophyllene oxide, terpineol, sabinene and/or cardinol.

According to an embodiment said therapeutic effect treats libido and/or sexual disfunction and said product comprises at least one of CBD, THC, CBC, CBG, CBN, CBL, THCV and CBDV. According to an embodiment said therapeutic effect treats libido and/or sexual disfunction and said product comprises CBD and optionally THC at CBD to THC weight/weight ratio greater than 1, greater than 2, greater than 3, greater than 4 or greater than 5. According to an embodiment said therapeutic effect treats libido and/or sexual disfunction and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats libido and/or sexual disfunction and said the primary terpene is selected from the group consisting of limonene, linalool, terpineol, geraniol, eucalyptol, pinene, citronellol and combinations thereof. According to an embodiment, the product comprises at least 2 of these terpenes, at least 3, at least 4, or at least 5 of the terpenes. According to an embodiment, the primary terpene is selected from the group consisting of limonene, linalool, ecualyptol and combinations thereof. According to various embodiments said primary terpene comprises terpineol, geraniol and/or pinene. According to an embodiment, said therapeutic effect treats libido and/or sexual disfunction and the product further comprises a compound selected from the group consisting of biochanin A, formonenetin, genistein, daidzein, glycitein, prenylnaringenin, diosgenin, acetin, cimicifugoside and combinations thereof.

According to an embodiment said therapeutic effect treats hair loss and/or thinning and said product comprises at least one of CBD, THC, CBC, CBG, CBN, CBL, THCV and CBDV. According to an embodiment said therapeutic effect treats hair loss and/or thinning and said product comprises CBD and optionally THC at CBD to THC weight/weight ratio greater than 1, greater than 2, greater than 3, greater than 4 or greater than 5. According to an embodiment said therapeutic effect treats hair loss and/or thinning and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats hair loss and/or thinning and said the primary terpene is selected from the group consisting of linalool, terpineol, eucalyptol, borneol, camphor, pinene, geraniol and combinations thereof. According to an embodiment, the product comprises at least 2 of these terpenes, at least 3, at least 4, or at least 5 of the terpenes. According to an embodiment, the primary terpene is selected from the group consisting of geraniol, eucalyptol, terpineol and combinations thereof. According to various embodiments said primary terpene comprises linalool, borneol, camphor and/or pinene.

According to an embodiment said therapeutic effect treats brittle nails and said product comprises at least one of CBD, THC, CBC, CBG, CBN, CBL, THCV and CBDV. According to an embodiment said therapeutic effect treats brittle nails and said product comprises CBD and optionally THC at CBD to THC weight/weight ratio greater than 1, greater than 2, greater than 3, greater than 4 or greater than 5. According to an embodiment said therapeutic effect treats brittle nails and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats brittle nails and said the primary terpene is selected from the group consisting of linalool, eucalyptol, geraniol, pinene, limonene and combinations thereof. According to an embodiment, the product comprises at least 2 of these terpenes, at least 3, at least 4, or all 5 of the terpenes. According to an embodiment, the primary terpene is selected from the group consisting of linalool, eucalyptol, limonene and combinations thereof. According to various embodiments said primary terpene comprises geraniol and/or pinene.

According to an embodiment said therapeutic effect treats irregular body odor and said product comprises at least one of CBD, THC, CBC, CBG, CBN, CBL, THCV and CBDV. According to an embodiment said therapeutic effect treats irregular body odor and said product comprises CBD and optionally THC at CBD to THC weight/weight ratio greater than 1, greater than 2, greater than 3, greater than 4 or greater than 5. According to an embodiment said therapeutic effect treats irregular body odor and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV.

According to an embodiment said therapeutic effect treats irregular body odor and said the primary terpene is selected from the group consisting of linalool, terpineol, geraniol, eucalyptol and combinations thereof. According to an embodiment, the product comprises at least 2 of these terpenes, at least 3, or all 4 of the terpenes. According to an embodiment, the primary terpene is selected from the group consisting of linalool, terpineol, geraniol and combinations thereof.

According to an embodiment said therapeutic effect treats Polycystic Ovary Syndrome (POCS), amenorrhea, oligomenorrhea, hypermenorrhea, metrorrhagia and said product comprises at least one of CBD, THC, CBC, CBG, CBN, CBL, THCV and CBDV. According to an embodiment said therapeutic effect treats Polycystic Ovary Syndrome (POCS), amenorrhea, oligomenorrhea, hypermenorrhea, metrorrhagia and said product comprises CBD and optionally THC at CBD to THC weight/weight ratio greater than 1, greater than 2, greater than 3, greater than 4 or greater than 5. According to an embodiment said therapeutic effect treats Polycystic Ovary Syndrome (POCS), amenorrhea, oligomenorrhea, hypermenorrhea, metrorrhagia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats Polycystic Ovary Syndrome (POCS), amenorrhea, oligomenorrhea, hypermenorrhea, metrorrhagia and said the primary terpene is selected from the group consisting of humulene, linalool, terpineol, caryophyllene, caryophyllene oxide, terpinene, citronellol, geraniol, thujone, camphor, viridiflorol, pinene, camphene, sabinene, myrcene, eucalyptol, carene, carotol, limonene, sabinene, cardinal, pinene and combinations thereof. According to an embodiment, the product comprises at least 2 of these terpenes, at least 3, at least 4, or at least 5 of the terpenes. According to an embodiment, the primary terpene is selected from the group consisting of linalool, eucalyptol, geraniol and combinations thereof. According to an embodiment, the primary terpene is selected from the group consisting of linalool, eucalyptol, caryophyllene, pinene and combinations thereof. According to various embodiments said primary terpene comprises humulene, terpineol, caryophyllene, caryophyllene oxide, terpinene, citronellol, thujone, camphor, viridiflorol, pinene, camphene, sabinene, myrcene, carene, carotol, limonene, sabinene, cardinal and/or pinene. According to an embodiment, said therapeutic effect treats Polycystic Ovary Syndrome (POCS), amenorrhea, oligomenorrhea, hypermenorrhea, metrorrhagia and the product further comprises a compound selected from the group consisting of linalyl acetate, geranyl acetate, eugenol, chamazulene, citronellyl formate and combinations thereof.

According to an embodiment said therapeutic effect treats premenstrual syndrome (PMS), premenstrual tension, and/or premenstrual dysphoric disorder (PMDD) and said product comprises at least one of CBD, THC, CBC, CBG, CBN, CBL, THCV and CBDV. According to an embodiment said therapeutic effect treats premenstrual syndrome (PMS), premenstrual tension, and/or premenstrual dysphoric disorder (PMDD) and said product comprises CBD and optionally THC at CBD to THC weight/weight ratio greater than 1, greater than 2, greater than 3, greater than 4 or greater than 5. According to an embodiment said therapeutic effect treats premenstrual syndrome (PMS), premenstrual tension, and/or premenstrual dysphoric disorder (PMDD) and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats premenstrual syndrome (PMS), premenstrual tension, and/or premenstrual dysphoric disorder (PMDD) and said the primary terpene is selected from the group consisting of limonene, linalool, eucalyptol, nerolidol, sabinene, pinene, caryophyllene, terpineol, geraniol, thujone, camphor, viridiflorol, champhene, myrcene, caryophyllene, terpinene, carene, cirtonellol, carotol, neral, geranial and combinations thereof. According to an embodiment, the product comprises at least 2 of these terpenes, at least 3, at least 4, or at least 5 of the terpenes. According to an embodiment, the primary terpene is selected from the group consisting of linalool, eucalyptol, geraniol and combinations thereof. According to an embodiment, the primary terpene is selected from the group consisting of linalool, caryophyllene, limonene and combinations thereof. According to an embodiment, the primary terpene is selected from the group consisting of linalool, caryophyllene, limonene, eucalyptol, geraniol and combinations thereof. According to various embodiments said primary terpene comprises limonene, nerolidol, sabinene, pinene, caryophyllene, terpineol, thujone, camphor, viridiflorol, champhene, myrcene, caryophyllene, terpinene, carene, cirtonellol, carotol, neral and/or geranial. According to an embodiment, said therapeutic effect treats premenstrual syndrome (PMS), premenstrual tension, and/or premenstrual dysphoric disorder (PMDD) and the product further comprises a compound selected from the group consisting of linalyl acetate, geranyl acetate, citronellyl formate, menthol, eugenol piperine, jasmone and combinations thereof.

According to an embodiment said therapeutic effect hormonal disfunction and/or imbalance and/or fertility and said product comprises at least one of CBD, THC, CBC, CBG, CBN, CBL, THCV and CBDV. According to an embodiment said therapeutic effect treats hormonal disfunction and/or imbalance and/or fertility and said product comprises CBD and optionally THC at CBD to THC weight/weight ratio greater than 1, greater than 2, greater than 3, greater than 4 or greater than 5. According to an embodiment said therapeutic effect treats hormonal disfunction and/or imbalance and/or fertility and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats hormonal disfunction and/or imbalance and/or fertility and said the primary terpene is selected from the group consisting of linalool, terpineol, geraniol, thujone, camphor, viridiflorol, borneol, champhene, pinene, eucalyptol, caryophyllene, germacrene, pinene, carene, cirtonellol, nerol, farnesol, limonene, myrcene, terpinene, menth-2-en-1-ol, carotol, sabinene, neral, geranial and combinations thereof. According to an embodiment, the product comprises at least 2 of these terpenes, at least 3, at least 4, or at least 5 of the terpenes. According to an embodiment, the primary terpene is selected from the group consisting of linalool, eucalyptol, geraniol and combinations thereof. According to an embodiment, the primary terpene is selected from the group consisting of linalool, eucalyptol, geraniol, terpineol and combinations thereof. According to an embodiment, the primary terpene is selected from the group consisting of linalool, eucalyptol, geraniol, pinene, caryophyllene and combinations thereof. According to various embodiments said primary terpene comprises terpineol, geraniol, thujone, camphor, viridiflorol, borneol, champhene, pinene, caryophyllene, germacrene, pinene, carene, cirtonellol, nerol, farnesol, limonene, myrcene, terpinene, menth-2-en-1-ol, carotol, sabinene, neral and/or geranial. According to an embodiment, said therapeutic effect treats hormonal disfunction and/or imbalance and/or fertility and the product further comprises a compound selected from the group consisting of linalyl acetate, geranyl acetate, chamazulene, citronellyl formate, biochanin A, formonenetin, genistein, daidzein, glycitein, prenylnaringenin, sclareol, diosgenin, acetin, cimicifugoside and combinations thereof.

According to an embodiment said therapeutic effect treats pre-eclampsia toxemia and said product comprises at least one of CBD, THC, CBC, CBG, CBN, CBL, THCV and CBDV. According to an embodiment said therapeutic effect treats pre-eclampsia toxemia and said product comprises CBD and optionally THC at CBD to THC weight/weight ratio greater than 1, greater than 2, greater than 3, greater than 4 or greater than 5. According to an embodiment said therapeutic effect treats pre-eclampsia toxemia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats pre-eclampsia toxemia and said the primary terpene is selected from the group consisting of limonene, neral, geranial, caryophyllene, linalool, myrcene, nerolidol, fenchol, humulene, guaiol, borneol, cymene, terpinene and combinations thereof. According to an embodiment, the product comprises at least 2 of these terpenes, at least 3, at least 4, or at least 5 of the terpenes. According to an embodiment, the primary terpene is selected from the group consisting of linalool, myrcene, limonene and combinations thereof. According to an embodiment, the primary terpene is selected from the group consisting of linalool, myrcene, limonene, caryophyllene and combinations thereof. According to various embodiments said primary terpene comprises neral, geranial, caryophyllene, nerolidol, fenchol, humulene, guaiol, borneol, cymene and/or terpinene.

According to an embodiment said therapeutic effect treats conditions and/or symptoms associated with female hormonal disfunction and/or imbalance and said product comprises at least one of CBD, THC, CBC, CBG, CBN, CBL, THCV and CBDV. According to an embodiment said therapeutic effect treats conditions and/or symptoms associated with female hormonal disfunction and/or imbalance and said product comprises CBD and optionally THC at CBD to THC weight/weight ratio greater than 1, greater than 2, greater than 3, greater than 4 or greater than 5. According to an embodiment said therapeutic effect treats conditions and/or symptoms associated with female hormonal disfunction and/or imbalance and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats conditions and/or symptoms associated with female hormonal disfunction and/or imbalance and said the primary terpene is selected from the group consisting of linalool, terpineol, geraniol, thujone, camphor, viridiflorol, borneol, champhene, pinene, eucalyptol, caryophyllene, germacrene, pinene, carene, cirtonellol, nerol, farnesol, limonene, myrcene, terpinene, menth-2-en-1-ol, carotol, sabinene, neral, geranial, nerolidol, terpineol, carene and combinations thereof. According to an embodiment, the product comprises at least 2 of these terpenes, at least 3, at least 4, or at least 5 of the terpenes. According to an embodiment, the primary terpene is selected from the group consisting of geraniol, linalool, eucalyptol and combinations thereof. According to an embodiment, the primary terpene is selected from the group consisting of terpineol, linalool, caryophyllene, pinene and combinations thereof. According to an embodiment, the primary terpene is selected from the group consisting of geraniol, linalool, eucalyptol, caryophyllene, terpineol, pinene, limonene and combinations thereof. According to various embodiments said primary terpene comprises terpineol, geraniol, thujone, camphor, viridiflorol, borneol, champhene, pinene, eucalyptol, caryophyllene, germacrene, pinene, carene, cirtonellol, nerol, farnesol, limonene, myrcene, terpinene, menth-2-en-1-ol, carotol, sabinene, neral, geranial, nerolidol, terpineol and/or carene. According to an embodiment, said therapeutic effect treats conditions and/or symptoms associated with female hormonal disfunction and/or imbalance and the product further comprises a compound selected from the group consisting of biochanin A, formonenetin, genistein, daidzein, glycitein, prenylnaringenin, diosgenin, acetin, cimicifugoside and combinations thereof.

According to an embodiment said therapeutic effect treats conditions and/or symptoms associated with perimenopause and/or menopause and said product comprises at least one of CBD, THC, CBC, CBG, CBN, CBL, THCV and CBDV. According to an embodiment said therapeutic effect treats conditions and/or symptoms associated with perimenopause and/or menopause and said product comprises CBD and optionally THC at CBD to THC weight/weight ratio greater than 1, greater than 2, greater than 3, greater than 4 or greater than 5. According to an embodiment said therapeutic effect treats conditions and/or symptoms associated with perimenopause and/or menopause and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats conditions and/or symptoms associated with perimenopause and/or menopause and said the primary terpene is selected from the group consisting of caryophyllene, caryophyllene oxide, carene, linalool, myrcene, terpinene, terpineol, citronellol, eucalyptol, nerolidol, limonene, geraniol, menthol, iso-menthone, germacrene, pinene, cardinol, neral, geranial, menth-2-en-1-ol, pulegone, cymene, fenchol, humulene, guaiol, borneol, carene, sabinene, myrtenol, camphene, nerol, farnesol, carotol and combinations thereof. According to an embodiment, the product comprises at least 2 of these terpenes, at least 3, at least 4, or at least 5 of the terpenes. According to an embodiment, the primary terpene is selected from the group consisting of caryophyllene, linalool, carene and combinations thereof. According to an embodiment, the primary terpene is selected from the group consisting of caryophyllene, linalool, myrcene and combinations thereof. According to an embodiment, the primary terpene is selected from the group consisting of nerolidol, limonene, linalool and combinations thereof. According to an embodiment, the primary terpene is selected from the group consisting of caryophyllene, linalool, carene, nerolidol, limonene and combinations thereof. According to various embodiments said primary terpene comprises caryophyllene, caryophyllene oxide, carene, myrcene, terpinene, terpineol, citronellol, eucalyptol, nerolidol, limonene, geraniol, menthol, iso-menthone, germacrene, pinene, cardinol, neral, geranial, menth-2-en-1-ol, pulegone, cymene, fenchol, humulene, guaiol, borneol, carene, sabinene, myrtenol, camphene, nerol, farnesol and/or carotol. According to an embodiment, said therapeutic effect treats conditions and/or symptoms associated with perimenopause and/or menopause and the product further comprises a compound selected from the group consisting of biochanin A, formonenetin, genistein, daidzein, glycitein, prenylnaringenin, diosgenin, acetin, cimicifugoside and combinations thereof.

According to an embodiment said therapeutic effect treats pain and/or cramps and said product comprises at least one of CBD, THC, CBC, CBG, CBN, CBL, THCV and CBDV. According to an embodiment said therapeutic effect treats pain and/or cramps and said product comprises CBD and optionally THC at CBD to THC weight/weight ratio greater than 1, greater than 2, greater than 3, greater than 4 or greater than 5. According to an embodiment said therapeutic effect treats pain and/or cramps and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats pain and/or cramps and said the primary terpene is selected from the group consisting of myrcene, linalool, caryophyllene, eucalyptol, nerolidol, sabinene, humulene, terpineol, citronellol, geraniol, pinene, limonene, camphene, terpinene, borneol, camphor, menthone, iso-menthone, carvacrol, thymol, carene, nerol, farnesol, menth-2-en-1-ol, guaiol and combinations thereof. According to an embodiment, the product comprises at least 2 of these terpenes, at least 3, at least 4, or at least 5 of the terpenes. According to an embodiment, the primary terpene is selected from the group consisting of limonene, linalool, eucalyptol and combinations thereof. According to an embodiment, the primary terpene is selected from the group consisting of limonene, linalool, eucalyptol, myrcene and combinations thereof. According to various embodiments said primary terpene comprises myrcene, caryophyllene, nerolidol, sabinene, humulene, terpineol, citronellol, geraniol, pinene, camphene, terpinene, borneol, camphor, menthone, iso-menthone, carvacrol, thymol, carene, nerol, farnesol, menth-2-en-1-ol and/or guaiol.

According to an embodiment said therapeutic effect treats female urogenital and/or reproductive system infections and/or disorders and said product comprises at least one of CBD, THC, CBC, CBG, CBN, CBL, THCV and CBDV. According to an embodiment said therapeutic effect treats female urogenital and/or reproductive system infections and/or disorders and said product comprises CBD and optionally THC at CBD to THC weight/weight ratio greater than 1, greater than 2, greater than 3, greater than 4 or greater than 5. According to an embodiment said therapeutic effect treats female urogenital and/or reproductive system infections and/or disorders and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats female urogenital and/or reproductive system infections and/or disorders and said the primary terpene is selected from the group consisting of myrcene, humulene, caryophyllene, linalool, terpineol, geraniol, pinene, germacrene, cirtonellol, limonene, myrcene, terpinene, menth-2-en-1-ol, borneol, eucalyptol, myrtenol, cymene, caryophyllene oxide, ocimene, nerolidol, camphor, sabinene, thujone, camphor, viridiflorol, menthol, iso-menthone, caryophyllene, germacrene, geraniol, nerol, neral, geranial, citronellol, carvacol, thymol, carene, myrtenol, carene, thujone and combinations thereof. According to an embodiment, the product comprises at least 2 of these terpenes, at least 3, at least 4, or at least 5 of the terpenes. According to an embodiment, the primary terpene is selected from the group consisting of linalool, pinene, caryophyllene oxide and combinations thereof. According to an embodiment, the primary terpene is selected from the group consisting of geraniol, linalool, pinene, caryophyllene oxide and combinations thereof. According to various embodiments said primary terpene comprises myrcene, humulene, caryophyllene, terpineol, geraniol, germacrene, cirtonellol, limonene, myrcene, terpinene, menth-2-en-1-ol, borneol, eucalyptol, myrtenol, cymene, ocimene, nerolidol, camphor, sabinene, thujone, camphor, viridiflorol, menthol, iso-menthone, caryophyllene, germacrene, geraniol, nerol, neral, geranial, citronellol, carvacol, thymol, carene, myrtenol, carene and/or thujone.

According to an embodiment, the product comprises an additive selected from the group consisting of antioxidants, emulsifiers and texturizers vegetable oils, plant extracts, honey, pharmaceutical excipients, sucrose, glucose and fructose, pharmaceutical excipients and combinations thereof. According to an embodiment, the product comprises a surfactant selected from the group consisting of phospholipids, glycerides, glycolipids and combinations thereof. According to an embodiment, the product further comprises a food-approved texturizer. According to an embodiment, the product further comprises at least 10 ppm ethanol. According to an embodiment, the product further comprises at least one of vitamin C, vitamin E, polyunsaturated fatty acids, beeswax and coconut oil. According to an embodiment, the product further comprises a sweetener. According to an embodiment, the product further comprises omega 3 fatty acid. According to an embodiment, the product further comprises omega 6 fatty acid. According to an embodiment, the product further comprises omega 3 fatty acid. According to an embodiment, the product further comprises curcumin.

According to an embodiment, the terpene-enriched cannabinoid product of improved therapeutic effect is a human medication. According to an embodiment, the terpene-enriched cannabinoid product of improved therapeutic effect is a veterinary medication.

According to an embodiment, the shelf life of the product is at least 6 months or at least a year. According to an embodiment, primary terpene degradation in the product is less than 20% per year.

According to an embodiment, further provided is a product selected from the group consisting of tablets, gel capsules, medical patches, topicals, creams, varnishes, sublingual oils, sprays, edibles, suppositories, tampons, rectal candle, cigarette, vaporizer liquid nasal preparation, preparations containing micro and nano-emulsions, preparations containing micro and nano-particles and combinations thereof, containing the product. According to an embodiment, further provided is a product comprising tablets containing the product. According to an embodiment, further provided is a product comprising gel capsules containing the product. According to an embodiment, further provided is a product comprising medical patches containing the product. According to an embodiment, further provided is a product comprising topicals containing the product. According to an embodiment, further provided is a product comprising creams containing the product. According to an embodiment, further provided is a product comprising varnishes containing the product. According to an embodiment, further provided is a product comprising sublingual oils containing the product. According to an embodiment, further provided is a product comprising edibles containing the product. According to an embodiment, further provided is a product comprising tampons containing the product. According to an embodiment, further provided is a product comprising rectal candles containing the product. According to an embodiment, further provided is a product comprising cigarettes containing the product. According to an embodiment, further provided is a product comprising vaporizer liquids containing the product.

According to an embodiment, further provided is a commercial product comprising two products according to the present invention, which two products differ in the content of the primary terpene. According to an embodiment, further provided is a commercial product comprising two products according to the present invention, which two products comprise different primary terpenes.

According to an embodiment, further provided is a method for producing the product comprising providing at least one cannabinoid and blending it with a primary terpene.

According to an embodiment, the method includes extracting cannabis plant material to form an extract. According to an embodiment the extracting comprises steam distillation. According to an embodiment, the method further comprises removing terpenes from the extract prior to the blending. According to an embodiment, the extracting comprises contacting with an extractant to form an extract, which extract comprises at least one cannabinoid and the extractant; and optionally removing at least a fraction of the extractant from the extract. According to an embodiment, the extractant comprises at least one of ethanol, a liquefied gas, such as butane, butene or dimethyl-ether, liquefied CO₂, near-critical CO₂ supercritical CO₂ and combinations thereof. According to an embodiment, extracting comprises contacting the cannabis plant material with an extractant to form an extract, and the extractant comprises at least one terpene. According to an embodiment, the extractant comprises the primary terpene.

According to an embodiment, the method further comprises at least partially decarboxylating the cannabinoid. According to an embodiment the decarboxylating is conducted at a temperature greater than 100° C. According to an embodiment the decarboxylating is conducted prior to extracting. According to an embodiment the decarboxylating is conducted on the extract prior to the blending with the primary terpene. According to an embodiment, a fraction of the extract is decarboxylated, and the decarboxylated fraction is blended with another fraction, which was not decarboxylated.

According to an embodiment, the method further comprises extracting a plant material, whereby the primary terpene is produced. According to an embodiment, the plant material is selected from the group including cannabis, lemons, oranges, hops, lavender, pine needles, Echinacea, tea, clover, capsicum, eucalyptus, geranium and mint.

According to an embodiment, the method comprises synthesizing at least one cannabinoid and blending the synthesized cannabinoid with the primary terpene.

According to an embodiment, the method further comprises blending cannabis plant material with the primary terpene. According to an embodiment, the cannabis plant material comprises a cannabis bud. According to an embodiment, the blending cannabis plant material with the primary terpene comprises spraying the cannabis plant material with the primary terpene, optionally in a solvent. According to an embodiment, the blending cannabis plant material with the primary terpene comprises combining the cannabis plant material with a substrate comprising the primary terpene. According to an embodiment, the substrate is a plant material sprayed with the primary terpene. According to an embodiment, the substrate is a cigarette paper sprayed with the primary terpene.

According to an embodiment, further provided is a method for producing the product comprising extracting cannabis plant material to form an extract, wherein the extracting forms at least two extract fractions, a cannabinoid-enriched extract and a terpene-enriched extract. As used herein, the term “cannabinoid-enriched extract” refers to an extract wherein cannabinoid to terpenes weight/weight ratio is greater than that in the plant material. As used herein, the term “terpene-enriched extract” refers to an extract wherein cannabinoid to terpenes weight/weight ratio is smaller than that in the plant material. According to an embodiment, the method comprises dividing the cannabinoid-enriched extract into at least two fractions and mixing at least one fraction with at least part of the terpene-enriched extract to form a terpene-enriched product, wherein terpenes to cannabinoid weight/weight ratio is greater than that ratio in the cannabis plant material. According to an embodiment the ratio is 1.5 times greater than that in the cannabis plant material, 2 times greater, 2.5 times greater, 3 times greater, 4 times greater, 5 times greater, 6 times greater, 7 times greater, 8 times greater, or 10 times greater. According to an embodiment, the terpene-enriched product is further mixed with at least one terpene.

According to an embodiment, further provided is a method for reaching, achieving and/or gaining a therapeutic effect in treating conditions and/or symptoms associated with perimenopause, menopause, female urogenital or reproductive system infections and/or disorders, menstrual cycle, hormonal deficiency and/or hormonal imbalance comprising administering to a subject in need a therapeutically effective amount of a product comprising (i) at least one cannabinoid in a specific amount, (ii) a primary terpene in a specific amount, (iii) at least 5% by weight of a non-cannabinoid, non-terpene, carrier; (iv) optionally at least three secondary terpenes; and (v) optionally at least one phytoestrogen, optionally combined with at least one herbal extract. According to an embodiment, the product is selected from the group consisting of cigarettes, vaporizer plant material, vaporizer liquid, extract, tablets, gel capsules, suppositories, tampons, rectal candle, cigarette, vaporizer liquid nasal preparation, preparations containing micro and nano-emulsions, preparations containing micro and nano-particles and combinations thereof. According to an embodiment, the daily dose of the product comprises about 1 milligram cannabinoid to about 300 milligram, about 2 milligram cannabinoid to about 200 milligram or about 3 milligram cannabinoid to about 100 milligram. According to another embodiment, the daily dose of the product comprises about 0.5 milligram cannabinoid per kilogram body weight to about 30 milligram cannabinoid per kilogram body weight.

According to an embodiment, the administered product comprises at least two cannabinoids, at least three, at least four or at least five. According to an embodiment, the content of each cannabinoid in the product is at least 10 parts per million (ppm). According to an embodiment, the cannabinoids are selected from the group consisting THC, CBD, CBG, CBC, CBN, CBL, THCV, CBDV and their non-decarboxylated form thereof.

According to an embodiment, the administered product comprises at least 5% by weight carrier, at least 10%, at least 15%, at least 20%, at least 25%, at least 30%, at least 35% or at least 40% by weight. Any compound other than cannabinoids and terpenes is a suitable carrier. According to an embodiment, the carrier is selected from the group consisting of vegetable oils, e.g. coconut oil, olive oil or sesame oil, pharmaceutical excipients, honey, bees wax, cellulose and combinations thereof.

According to an embodiment, the administered product comprises at least one phytoestrogen. According to an embodiment, the product comprises at least two, at least three, at least four, or at least five phytoestrogens.

According to an embodiment, the administered product comprises a primary terpene and optionally at least three secondary terpenes, at least four or at least five. According to an embodiment, the primary terpene forms at least 20% by weight of the total amount of terpenes in the product, at least 30%, at least 40%, at least 50%, at least 60%, at least 70% or at least 80%. According to an embodiment, the product comprises multiple (e.g. two or three) terpenes, each one of which forms at least 20% by weight of the total amount of terpenes in the product and each one of these terpenes is considered a primary terpene. According to an embodiment, the content of the primary terpene in the product is at least 2 times greater than that of any secondary terpene, at least 4, at least 6, at least 8, at least 10, at least 15, at least 20, at least 25, or at least 30 times greater.

According to an embodiment, the administered product is combines with at least one herbal extract, at least two, at least three, at least four, or at least five herbal extracts.

According to an embodiment, the administered product comprises less than 5% by weight glycol, less than 4%, less than 3%, less than 2%, or less than 1% by weight.

According to an embodiment, the administered product comprises less than 20% by weight water, less than 15% by weight, less than 14%, less than 13%, less than 12%, less than 11% or less than 10% by weight.

According to a related embodiment, the administered product comprises chlorophyll. According to an embodiment, the product comprises at least 0.5% by weight chlorophyll, at least 1%, at least 5%, at least 10%, at least 15%, at least 20% chlorophyll. According to an embodiment, the administered product comprises at least one flavonoid. According to an embodiment, the product comprises at least two, at least three, at least four, or at least five flavonoids. According to an embodiment, the administered product comprises at least one of isoflavones, coumestans, prenylflavonoids, lignans and saponins. According to an embodiment, the product comprises isoflavones. According to an embodiment, the product comprises coumestans. According to an embodiment, the product comprises prenylflavonoids. According to an embodiment, the product comprises lignans. According to an embodiment, the product comprises saponins. According to an embodiment, the product comprises at least one of biochanin A, formonenetin, genistein, daidzein, glycitein, prenylnaringenin, diosgenin, acetin and/or cimicifugoside. According to an embodiment, the product comprises biochanin A. According to an embodiment, the product comprises formonenetin. According to an embodiment, the product comprises genistein. According to an embodiment, the product comprises daidzein. According to an embodiment, the product comprises glycitein. According to an embodiment, the product comprises prenylnaringenin. According to an embodiment, the product comprises diosgenin. According to an embodiment, the product comprises acetin. According to an embodiment, the product comprises cimicifugoside.

According to an embodiment, the non-cannabinoid, non-terpene carrier comprises cellulose and the terpenes to cannabinoids weight/weight ratio in the product is about 0.1 to about 1.0. According to an embodiment said non-cannabinoid, non-terpene, carrier comprises at least 5% by weight cellulose, at least 6%, at least 8%, at least 10%, at least 15%, or at least 20%. According to an embodiment, the ratio is greater than 0.1, greater than 0.15, greater than 0.2, greater than 0.25, greater than 0.3, greater than 0.35, greater than 0.4, greater than 0.45, greater than 0.5, greater than 0.55, greater than 0.6, greater than 0.65, greater than 0.7, greater than 0.75, greater than 0.8, greater than 0.85, greater than 0.9, greater than 0.95, greater than 1, greater than 1.2, greater than 1.5, greater than 2.0, greater than 3 or greater than 5. According to an embodiment, the ratio is less than 0.9, less than 0.8, less than 0.7, less than 0.6, less than 0.5, less than 0.4, less than 0.3, less than 0.2 or less than 0.15. According to an embodiment, the product comprising more than 5% cellulose is selected from the group consisting of cannabis plant material, e.g. cannabis buds or cannabis trim, ground forms thereof, plant material preparation for vaporizers and cannabis cigarette.

According to an embodiment, the non-cannabinoid, non-terpene carrier comprises less than 5% by weight cellulose and the terpenes to cannabinoids weight/weight ratio in the product is about 0.05 to about 1.0. According to an embodiment said non-cannabinoid, non-terpene, carrier comprises less than 5% by weight cellulose, less than 4%, less than 3%, less than 2%, or less than 1% cellulose. According to an embodiment, the ratio is greater than 0.1, greater than 0.15, greater than 0.2, greater than 0.25, greater than 0.3, greater than 0.35, greater than 0.4, greater than 0.45, greater than 0.5, greater than 0.55, greater than 0.6, greater than 0.65, greater than 0.7, greater than 0.75, greater than 0.8, greater than 0.85, greater than 0.9, greater than 0.95, greater than 1, greater than 1.2, greater than 1.5, greater than 2.0, greater than 3 or greater than 5. According to an embodiment, the ratio is less than 0.9, less than 0.8, less than 0.7, less than 0.6, less than 0.5, less than 0.4, less than 0.3, less than 0.2, less than 0.15 or less than 0.1. According to an embodiment, the product comprising less than 5% cellulose is selected from the group consisting of cannabis trichomes, cannabis extracts and products thereof, such as tablets, gel capsules, medical patches, vaporizer liquids and suppositories, tampons, rectal candle, cigarette, vaporizer liquid nasal preparation, preparations containing micro and nano-emulsions, preparations containing micro and nano-particles and combinations thereof.

According to an embodiment, the conditions and/or symptoms associated with menopause are selected from the group consisting of hot flashes, facial flushing, night sweat, osteoporosis, fatigue, irritability, insomnia and/or sleep disorder, difficulty in concentration, depression, memory loss, brain fog, headache, migraine, anxiety, nervousness, restless, stress, mood swing, affect disorder, general discomfort, muscle tension, myalgia, intermittent dizziness, paresthesia, indigestion, digestive problems, intestinal disorders, abdominal pain, irritability, palpitations, tachycardia, nausea, constipation, diarrhea, arthralgia and/or arthritis, dysmenorrheal, pelvic pain, spasms, cramps, labor contraction, endometriosis Polycystic Ovary Syndrome (POCS), amenorrhea, oligomenorrhea, hypermenorrhea, metrorrhagia, premenstrual syndrome (PMS), premenstrual tension, premenstrual dysphoric disorder (PMDD), cold hands and feet, weight gain, mental awareness problems, sexual pain disorder, dyspareunia, breast pain and/or mastalgia, menopausal skin, inflammation, oxidative stress, cardiovascular disease, loss of pelvic muscle tone, urinary incontinence, lower urinary tract disorders, vaginal dryness, women urogenital and/or reproductive system infection, fibroid, myomas and/or cysts in the female genital system, human papillomavirus (HPV), Uterine Fibroid, cystitis, urethritis, pyelonephritis, women hormonal dysfunction and/or imbalance, irregular periods, pre-eclampsia toxemia, reduced libido, reduced sexual dysfunction, hair loss and/or thinning, age-related winkles, stretch marks, brittle nails, gam disorders, irregular body odor, conditions and/or symptoms induced by estrogen deficiency and combinations thereof.

According to an embodiment, the administered product additionally contains an additive selected from the group consisting of antioxidants, emulsifiers and texturizers vegetable oils, plant extracts, honey, sucrose, glucose and fructose, pharmaceutical excipients and combinations thereof.

According to an embodiment, the non-cannabinoid, non-terpene, carrier comprises cellulose, and the administered product comprises (a) tetrahydrocannabinol (THC) and/or tetrahydrocannabinolic acid (THCa) in a total concentration of at least 5% by weight; (b) cannabidiol (CBD) and/or cannabidiolic acid (CBDa) in a total concentration of at least 5% by weight; (c) tetrahydrocannabinol (THC) and/or tetrahydrocannabinolic acid (THCa) in a total concentration of at least 2.5% by weight, and cannabidiol (CBD) and/or cannabidiolic acid (CBDa) in a total concentration of at least 2.5% by weight; or (d) at least one of cannabigerol (CBG), cannabinol (CBN), cannabichromene (CBC), and cannabicyclol (CBL), tetrahydrocannabivarin (THCV), cannabidivarin (CBDV), and non-decarboxylated form thereof in a concentration of at least 0.5% by weight; and (i) the primary terpene forms at least 40% by weight of the total terpene content, and (ii) the primary terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thujone, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

According to a related embodiment said non-cannabinoid, non-terpene, carrier comprises at least 5% by weight cellulose, at least 6%, at least 8%, at least 10%, at least 15%, or at least 20%.

According to a related embodiment said tetrahydrocannabinol (THC) and/or tetrahydrocannabinolic acid (THCa) is in a total concentration of at least 4% by weight, at least 5%, at least 6%, at least 8%, at least 10%, at least 12%, at least 14%, at least 16%, at least 18%, or at least 20% by weight.

According to a related embodiment said cannabidiol (CBD) and/or cannabidiolic acid (CBDa) is in a total concentration of at least 4% by weight, at least 5%, at least 6%, at least 8%, at least 10%, at least 12%, at least 14%, at least 16%, at least 18% or at least 20% by weight.

According to a related embodiment, said administered product comprises tetrahydrocannabinol (THC) and/or tetrahydrocannabinolic acid (THCa) in a total concentration of at least 2.5% by weight, and cannabidiol (CBD) and/or cannabidiolic acid (CBDa) in a total concentration of at least 2.5% by weight; tetrahydrocannabinol (THC) and/or tetrahydrocannabinolic acid (THCa) in a total concentration of at least 3% by weight, and cannabidiol (CBD) and/or cannabidiolic acid (CBDa) in a total concentration of at least 3% by weight; tetrahydrocannabinol (THC) and/or tetrahydrocannabinolic acid (THCa) in a total concentration of at least 4% by weight, and cannabidiol (CBD) and/or cannabidiolic acid (CBDa) in a total concentration of at least 4% by weight; tetrahydrocannabinol (THC) and/or tetrahydrocannabinolic acid (THCa) in a total concentration of at least 5% by weight, and cannabidiol (CBD) and/or cannabidiolic acid (CBDa) in a total concentration of at least 5% by weight; tetrahydrocannabinol (THC) and/or tetrahydrocannabinolic acid (THCa) in a total concentration of at least 8% by weight, and cannabidiol (CBD) and/or cannabidiolic acid (CBDa) in a total concentration of at least 8% by weight; tetrahydrocannabinol (THC) and/or tetrahydrocannabinolic acid (THCa) in a total concentration of at least 10% by weight, and cannabidiol (CBD) and/or cannabidiolic acid (CBDa) in a total concentration of at least 10% by weight.

According to a related embodiment, said administered product comprises cannabigerol (CBG) and/or cannabigerol acid (CBGa) in a total concentration of at least 0.1% by weight, at least 0.5%, at least 1%, at least 2%, at least 3%, at least 4%, or at least 5% by weight. According to a related embodiment, said product comprises cannabinol (CBN) and/or cannabinol acid (CBNa) in a total concentration of at least 0.1% by weight, at least 0.5%, at least 1%, at least 2%, at least 3%, at least 4%, or at least 5% by weight. According to a related embodiment, said product comprises cannabichromene (CBC) and/or cannabichromenic acid (CBCa) in a total concentration of at least 0.1% by weight, at least 0.5%, at least 1%, at least 2%, at least 3%, at least 4%, or at least 5% by weight. According to a related embodiment, said product comprises cannabicyclol (CBL) and/or cannabicyclol acid (CBLa) in a total concentration at least 0.1% by weight, at least 0.5%, at least 1%, at least 2%, at least 3%, at least 4%, or at least 5% by weight. According to a related embodiment, said product comprises tetrahydrocannabivarin (THCV), and/or tetrahydrocannabivarin acid (THCVA) in a total concentration at least 0.1% by weight, at least 0.5%, at least 1%, at least 2%, at least 3%, at least 4%, or at least 5% by weight. According to a related embodiment, said product comprises cannabidivarin (CBDV) and/or cannabigerovarin acid (CBGVA) in a total concentration at least 0.1% by weight, at least 0.5%, at least 1%, at least 2%, at least 3%, at least 4%, or at least 5% by weight.

According to a related embodiment, said primary terpene forms at least 20% by weight of the total terpene content, at least 30%, at least 40%, at least 50%, at least 60%, at least 70%, at least 80% or at least 90% by weight of the total terpene content. According to a related embodiment, said primary terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thujone, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol and combinations thereof. According to a related embodiment, said product comprises a dried cannabis plant material. According to a related embodiment, said product comprises ground cannabis plant material, is a vaporizer cannabis filling material or is a cannabis cigarette.

According to an embodiment, the administered product comprises (a) at least one cannabinoid from the group consisting of THC, CBD, CBG, CBC, CBN, CBL, THCV, CBDV and their non-decarboxylated form thereof; (b) a non-cannabinoid, non-terpene carrier, comprising less than 5% by weight cellulose; (c) a primary terpene, forming at least 40% by weight of the total terpene content, wherein the primary terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thujone, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

According to a related embodiment said THC and/or THCa is in a total concentration of at least 1% by weight, at least 2%, at least 3%, at least 4%, at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to a related embodiment said CBD and/or CBDa is in a total concentration of at least 1% by weight, at least 2%, at least 3%, at least 4%, at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to a related embodiment said CBG and/or CBGa is in a total concentration of at least 0.1% by weight, at least 0.5%, at least 1%, at least 2%, at least 3%, at least 4%, at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to a related embodiment said CBC and/or CBCa is in a total concentration of at least 0.1% by weight, at least 0.5%, at least 1%, at least 2%, at least 3%, at least 4%, at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to a related embodiment said CBN and/or CBNa is in a total concentration of at least 0.1% by weight, at least 0.5%, at least 1%, at least 2%, at least 3%, at least 4%, at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to a related embodiment said CBL and/or CBLa is in a total concentration of at least 0.1% by weight, at least 0.5%, at least 1%, at least 2%, at least 3%, at least 4%, at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to a related embodiment said THCV and/or THCVa is in a total concentration of at least 0.1% by weight, at least 0.5%, at least 1%, at least 2%, at least 3%, at least 4%, at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to a related embodiment said CBDV and/or CBDVa is in a total concentration of at least 0.1% by weight, at least 0.5%, at least 1%, at least 2%, at least 3%, at least 4%, at least 5%, at least 6%, at least 8%, at least 10%, at least 12%, at least 14%, at least 16%, at least 18% or at least 20%, at least 30%, at least 40%, at least 50%, at least 60%, at least 70%, at least 80%, or at least 90% by weight.

According to a related embodiment said non-cannabinoid non-terpene carrier comprises less than 5% by weight cellulose, less than 4%, less than 3%, less than 2%, or less than 1%, or less than 0.5% by weight cellulose.

According to a related embodiment, said primary terpene forms at least 20% by weight of the total terpene content, at least 30%, at least 40%, at least 50%, at least 60%, at least 70%, at least 80% or at least 90% by weight of the total terpene content. According to a related embodiment, said primary terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thujone, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol and combinations thereof. According to an embodiment, said administered product is liquid. According to an embodiment, said administered product is selected from the group consisting of vaporizer cannabis filling liquid, cannabis tablets, cannabis suppositories, tampons, rectal candle, cigarette, vaporizer liquid nasal preparation, preparations containing micro and nano-emulsions, preparations containing micro and nano-particles, cannabis gel capsules, cannabis candies, cannabis drinks and cannabis baked products.

According to an embodiment, the primary terpene of the administered product is selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thujone, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

According to an embodiment, the product results in an increased therapeutic effect compared with that of a product comprising the same cannabinoids amounts and a smaller amount of the primary terpene, e.g. one half of that amount. According to various embodiment, the increased therapeutic effect has various forms, e.g. a shorter onset time, increased magnitude, extended duration, reduced dosages, reduced secondary adverse symptoms, reduced frequency of conditions and/or symptoms, reduced severity of conditions and/or symptoms, reduced consumption of other drugs and combinations thereof. According to an embodiment, the increased therapeutic effect comprises a shorter onset time, or differently put an earlier effect, which is important particularly in cases of sublingual and topical delivery and in cases where a rapid effect is desired, as in treating pain. According to an embodiment, the increased therapeutic effect comprises extended duration of the therapeutic effect, for example an extended time of pain relief. According to an embodiment, the increased therapeutic effect comprises increased magnitude of the therapeutic effect, enabling achieving a desired therapeutic effect on administering smaller doses of cannabinoids, saving thereby on cost. According to an embodiment, the increased therapeutic effect comprises using smaller doses of cannabinoids and still achieving at least the same beneficial result. According to an embodiment, the increased therapeutic effect comprises reduction of secondary adverse symptoms, e.g. adverse symptoms of the main illness, of ones of another illness and/or ones related to administered the product or other drugs. According to an embodiment, the increased therapeutic effect comprises reduced frequency of the conditions and/or symptoms. According to an embodiment, the increased therapeutic effect comprises reduced severity of the conditions and/or symptoms.

According to an embodiment, the primary terpene and the cannabinoids are present in specific amounts, and the onset time of the therapeutic effect, as measured by methods known in the art, is at least 20% shorter than that of administering a product comprising the same cannabinoids amounts and one half the amount of the primary terpene, at least 30%, at least 40%, at least 50%, at least 60%, or at least 70% shorter. According to an embodiment, the primary terpene, secondary terpenes and the cannabinoids are present in specific amounts, and the onset time of the therapeutic effect is at least 20% shorter than that of administering a product comprising the same cannabinoids amounts, same secondary terpene amounts and one half the amount of the primary terpene, at least 30%, at least 40%, at least 50%, at least 60%, or at least 70% short. Shorter onset time, or differently put an earlier effect, is important particularly in cases of sublingual and topical delivery and in cases where a rapid effect is desired, as in treating pain.

According to an embodiment, the primary terpene and the cannabinoids are present in specific amounts, and the onset time of the therapeutic effect is at least 20% longer than that of administering a product comprising the same cannabinoids amounts and one half the amount of the primary terpene, at least 30%, at least 40%, at least 50%, at least 60%, or at least 70% longer. According to an embodiment, the primary terpene, secondary terpenes and the cannabinoids are present in specific amounts, and the onset time of the therapeutic effect is at least 20% longer than that of administering a product comprising the same cannabinoids amounts, same secondary terpene amounts and one half the amount of the primary terpene, at least 30%, at least 40%, at least 50%, at least 60%, or at least 70% longer. According to an embodiment, products of delayed onset time are used in combination with shorter onset time to reach a sustained release effect.

According to an embodiment, the primary terpene and the cannabinoids are present in specific amounts, and the magnitude of the therapeutic effect, as measured by methods known in the art, is at least 20% greater compared with that of administering a product comprising the same cannabinoids amounts and one half the amount of the primary terpene, at least 30%, at least 40%, at least 50%, at least 60%, or at least 70% greater. According to an embodiment, the primary terpene, secondary terpenes and the cannabinoids are present in specific amounts, and the magnitude of the therapeutic effect is at least 20% greater than that of administering a product comprising the same cannabinoids amounts, same secondary terpene amounts and one half the amount of the primary terpene, at least 30%, at least 40%, at least 50%, at least 60%, or at least 70% greater. Without wishing to be limited by any particular theory, such increased magnitude of the therapeutic effect may indicate increased bioavailability. Such increased magnitude enables achieving a desired therapeutic effect on administering smaller doses of cannabinoids, saving thereby on cost.

According to an embodiment, the primary terpene and the cannabinoids are present in specific amounts, and the duration of the therapeutic effect, as measured by methods known in the art, is at least 20% longer compared with that of administering a product comprising the same cannabinoids amounts and one half the amount of the primary terpene, at least 30%, at least 40%, at least 50%, at least 60%, at least 70%, at least 80%, at least 90% or at least 100% longer. According to an embodiment, the primary terpene, secondary terpenes and the cannabinoids are present in specific amounts, and the duration of the therapeutic effect is at least 20% longer than that of administering a product comprising the same cannabinoids amounts, same secondary terpene amounts and one half the amount of the primary terpene, at least 30%, at least 40%, at least 50%, at least 60%. at least 70%, at least 80%, at least 900% or at least 100% longer.

According to an embodiment, the primary terpene and the cannabinoids are present in specific amounts, and the frequency of the conditions and/or symptoms, as measured by methods known in the art, is at least 20% smaller compared with that of administering a product comprising the same cannabinoids amounts and one half the amount of the primary terpene, at least 30%, at least 40%, at least 50% at least 60%. at least 70%, at least 80%, or at least 90% smaller. According to an embodiment, the primary terpene, secondary terpenes and the cannabinoids are present in specific amounts, and the frequency of the conditions and/or symptoms, as measured by methods known in the art, is at least 20% smaller than that of administering a product comprising the same cannabinoids amounts, same secondary terpene amounts and one half the amount of the primary terpene, at least 30%, at least 40%, at least 50%, at least 60%. at least 70%, at least 80%, at least 90% or at least 100% longer.

According to an embodiment, the primary terpene and the cannabinoids are present in specific amounts, and the severity of the conditions and/or symptoms, as measured by methods known in the art, is at least 20% smaller compared with that of administering a product comprising the same cannabinoids amounts and one half the amount of the primary terpene, at least 300%, at least 40%, at least 50% at least 60%, at least 70%, at least 80%, or at least 90% smaller. According to an embodiment, the primary terpene, secondary terpenes and the cannabinoids are present in specific amounts, and the severity of the conditions and/or symptoms, as measured by methods known in the art, is at least 20% smaller than that of administering a product comprising the same cannabinoids amounts, same secondary terpene amounts and one half the amount of the primary terpene, at least 30%, at least 40%, at least 50%, at least 60%, at least 70%, at least 80%, at least 90% or at least 100% smaller.

According to an embodiment, the primary terpene and the cannabinoids are present in specific amounts, and the consumption of other drugs is reduced by at least 20% compared with that of administering a product comprising the same cannabinoids amounts and one half the amount of said primary terpene, reduced by at least 30%, by at least 40%, by at least 50%, by at least 60%, by at least 70%, by at least 80%, or by at least 90%. According to an embodiment, the primary terpene, secondary terpenes and the cannabinoids are present in specific amounts, and the consumption of other drugs, as measured by methods known in the art, is reduced by at least 20% compared with administering a product comprising the same cannabinoids amounts, same secondary terpene amounts and one half the amount of the primary terpene, reduced by at least 30%, by at least 40%, by at least 50%, by at least 60%, by at least 70%, by at least 80%, or by at least 900%.

According to an embodiment said therapeutic effect treats osteoporosis and said administered product comprises at least one of CBD, THC, CBC, CBG, CBN, CBL, THCV and CBDV. According to an embodiment said therapeutic effect treats osteoporosis and said product comprises CBD and optionally THC at CBD to THC weight/weight ratio greater than 1, greater than 2, greater than 3, greater than 4 or greater than 5. According to an embodiment said therapeutic effect treats osteoporosis and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats osteoporosis and said the primary terpene is selected from the group consisting of caryophyllene, caryophyllene oxide, carene, eucalyptol, sabinene, pinene, limonene, borneol, thymol, camphor and combinations thereof. According to an embodiment, the product comprises at least 2 of these terpenes, at least 3, at least 4, or at least 5 of the terpenes. According to an embodiment, the primary terpene is selected from the group consisting of caryophyllene, carene, borneol and combinations thereof. According to various embodiments said primary terpene comprises caryophyllene oxide, eucalyptol, sabinene, pinene, limonene, thymol and/or camphor. According to an embodiment, said therapeutic effect treats osteoporosis and the product further comprises a compound selected from the group consisting of biochanin A, formonenetin, genistein, daidzein, glycitein, prenylnaringenin, diosgenin, acetin, cimicifugoside and combinations thereof. According to an embodiment, said therapeutic effect treats osteoporosis and the product further comprises an herbal extract selected from the group consisting of angelica sinensis, cimicifuga (Actaea) racemosa, dioscorea villosa, trifolium pratense and combinations thereof.

According to an embodiment said therapeutic effect treats sleep disorder and/or insomnia and said administered product comprises at least one of CBD, THC, CBC, CBG, CBN, CBL, THCV and CBDV. According to an embodiment said therapeutic effect treats sleep disorder and/or insomnia and said product comprises CBD and optionally THC at CBD to THC weight/weight ratio greater than 1, greater than 2, greater than 3, greater than 4 or greater than 5. According to an embodiment said therapeutic effect treats sleep disorder and/or insomnia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats sleep disorder and/or insomnia and said the primary terpene is selected from the group consisting of linalool, caryophyllene, myrcene, terpineol, terpinene, citronellol, eucalyptol, nerolidol, sabinene, pinene, limonene, borneol, fenchol, bisabolol humulene, phytol and combinations thereof. According to an embodiment, the product comprises at least 2 of these terpenes, at least 3, at least 4, or at least 5 of the terpenes. According to an embodiment, the primary terpene is selected from the group consisting of linalool, caryophyllene, myrcene and combinations thereof. According to an embodiment, the primary terpene is selected from the group consisting of linalool, nerolidol, myrcene and combinations thereof. According to various embodiments said primary terpene comprises terpineol, terpinene, citronellol, eucalyptol, nerolidol, sabinene, pinene, limonene, borneol, fenchol, bisabolol, phytol and/or humulene. According to an embodiment, said therapeutic effect treats sleep disorder and/or insomnia and the product further comprises a compound selected from the group consisting of linalyl acetate, chamazulene, biochanin A, formonenetin, genistein, daidzein, glycitein, prenylnaringenin, diosgenin, acetin, cimicifugoside and combinations thereof. According to an embodiment, said therapeutic effect treats sleep disorder and/or insomnia and the product further comprises an herbal extract selected from the group consisting of avena sativa, cimicifuga (Actaea) racemosa, humulus lupulus, hypericum perforatum, lavendula officinalis, melissa officinalis, passiflora incarnata, valeriana officinalis and combinations thereof. According to an embodiment, the product includes caryophyllene, and caryophyllene forms less than 5% by weight of the total terpene content.

According to an embodiment said therapeutic effect treats anxiety, irritability, nervousness, restless, stress, depression, mood swing and/or affect disorder and said administered product comprises at least one of CBD, THC, CBC, CBG, CBN, CBL, THCV and CBDV. According to an embodiment said therapeutic effect treats anxiety, irritability, nervousness, restless, stress, depression, mood swing and/or affect disorder and said product comprises CBD and optionally THC at CBD to THC weight/weight ratio greater than 1, greater than 2, greater than 3, greater than 4 or greater than 5. According to an embodiment said therapeutic effect treats anxiety, irritability, nervousness, restless, stress, depression, mood swing and/or affect disorder and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV.

According to an embodiment said therapeutic effect treats anxiety, irritability, nervousness, restless, stress, depression, mood swing and/or affect disorder and said the primary terpene is selected from the group consisting of limonene, linalool, terpineol, citronellol, eucalyptol, caryophyllene, geraniol, menthol, iso-menthone, germacrene, pinene, citronellol, cardinol, neral, geranial, myrcene, terpinene, menth-2-en-1-ol, humulene, bisabolol, borneol, fenchol, nerolidol and combinations thereof. According to an embodiment, the product comprises at least 2 of these terpenes, at least 3, at least 4, or at least 5 of the terpenes. According to an embodiment, the primary terpene is selected from the group consisting of limonene, linalool, eucalyptol and combinations thereof. According to an embodiment, the primary terpene is selected from the group consisting of limonene, linalool, caryophyllene, bisabolol and combinations thereof. According to an embodiment, the primary terpene is selected from the group consisting of limonene, linalool, pinene, humulene and combinations thereof. According to various embodiments said primary terpene comprises terpineol, citronellol, caryophyllene, geraniol, menthol, iso-menthone, germacrene, pinene, citronellol, cardinol, neral, geranial, myrcene, terpinene, menth-2-en-1-ol, humulene, bisabolol, borneol, nerolidol or fenchol. According to an embodiment, said therapeutic effect treats anxiety, irritability, nervousness, restless, stress, depression, mood swing and/or affect disorder and the product further comprises a compound selected from the group consisting of linalyl acetate, geranyl acetate, menthol, piperine, eugenol, citronellyl formate, jasmone, benzy acetate, benzyl alcohol, biochanin A, formonenetin, genistein, daidzein, glycitein, prenylnaringenin, diosgenin, acetin, cimicifugoside and combinations thereof. According to an embodiment, said therapeutic effect treats treats anxiety, irritability, nervousness, restless, stress, depression, mood swing and/or affect disorder and the product further comprises an herbal extract selected from the group consisting of avena sativa, bacopa monnieri, centella (hydrocotyl) asiatica, humulus lupulus, hypericum perforatum, melissa officinalis, passiflora incarnata, valeriana officinalis and combinations thereof. According to an embodiment, the product includes pinene, terpineol, bisabolol, caryophyllene, myrcene and/or limonene, and pinene, terpineol, bisabolol, caryophyllene, myrcene and/or limonene forms less than 5% by weight of the total terpene content.

According to an embodiment said therapeutic effect treats difficulty in concentrating, brain fog and/or memory loss and said administered product comprises at least one of CBD, THC, CBC, CBG, CBN, CBL, THCV and CBDV. According to an embodiment said therapeutic effect treats difficulty in concentrating, brain fog and/or memory loss and said product comprises CBD and optionally THC at CBD to THC weight/weight ratio greater than 1, greater than 2, greater than 3, greater than 4 or greater than 5. According to an embodiment said therapeutic effect treats difficulty in concentrating, brain fog and/or memory loss and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats difficulty in concentrating, brain fog and/or memory loss and said the primary terpene is selected from the group consisting of eucalyptol, pinene, carene, limonene, pulegone, terpineol, cymene, linalool, caryophyllene and combinations thereof. According to an embodiment, the product comprises at least 2 of these terpenes, at least 3, at least 4, or at least 5 of the terpenes. According to an embodiment, the primary terpene is selected from the group consisting of eucalyptol, pinene, limonene and combinations thereof. According to various embodiments said primary terpene comprises carene, pulegone, terpineol, linalool, caryophyllene and/or cymene According to an embodiment, said therapeutic effect treats difficulty in concentrating, brain fog and/or memory loss and the product further comprises a compound selected from the group consisting of biochanin A, formonenetin, genistein, daidzein, glycitein, prenylnaringenin, diosgenin, acetin, cimicifugoside and combinations thereof. According to an embodiment, said therapeutic effect treats brain fog and/or memory loss and the product further comprises an herbal extract selected from the group consisting of asphalentum ponjabianum—mumio, bacopa monnieri, centella (hydrocotyl) asiatica, eleutherococcus senticosus, ginkgo biloba, lepidium meyenii, lycium barbarum, paullinia cupana, rhodiola rosea and combinations thereof.

According to an embodiment said therapeutic effect treats general discomfort and said administered product comprises at least one of CBD, THC, CBC, CBG, CBN, CBL, THCV and CBDV. According to an embodiment said therapeutic effect treats general discomfort and said product comprises CBD and optionally THC at CBD to THC weight/weight ratio greater than 1, greater than 2, greater than 3, greater than 4 or greater than 5. According to an embodiment said therapeutic effect treats general discomfort and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats general discomfort and said the primary terpene is selected from the group consisting of limonene, linalool humulene, terpinene, borneol, caryophyllene and combinations thereof. According to an embodiment, the product comprises at least 2 of these terpenes, at least 3, at least 4, or at least 5 of the terpenes. According to an embodiment, the primary terpene is selected from the group consisting of linalool, humulene, terpinene and combinations thereof. According to various embodiments said primary terpene comprises limonene, borneol and/or caryophyllene. According to an embodiment, the product includes myrcene, pinene and/or bisabolol, and myrcene, pinene and/or bisabolol forms less than 5% by weight of the total terpene content.

According to an embodiment said therapeutic effect treats muscle tension and/or myalgia and said administered product comprises at least one of CBD, THC, CBC, CBG, CBN, CBL, THCV and CBDV. According to an embodiment said therapeutic effect treats muscle tension and/or myalgia and said product comprises CBD and optionally THC at CBD to THC weight/weight ratio greater than 1, greater than 2, greater than 3, greater than 4 or greater than 5. According to an embodiment said therapeutic effect treats muscle tension and/or myalgia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats muscle tension and/or myalgia and said the primary terpene is selected from the group consisting of myrcene, pinene, nerolidol, linalool, humulene, caryophyllene, limonene, bisabolol and combinations thereof. According to an embodiment, the product comprises at least 2 of these terpenes, at least 3, at least 4, or at least 5 of the terpenes. According to an embodiment, the primary terpene is selected from the group consisting of myrcene, pinene, nerolidol and combinations thereof. According to an embodiment, the primary terpene is selected from the group consisting of myrcene, pinene, nerolidol, linalool, humulene and combinations thereof. According to various embodiments said primary terpene comprises linalool, humulene, caryophyllene, limonene and/or bisabolol. According to an embodiment, said therapeutic effect treats muscle tension and/or myalgia and the product further comprises an herbal extract selected from the group consisting of boswellia spp, salix alba, valeriana edulis, viburnum opulus, chamaemelum and combinations thereof.

According to an embodiment said therapeutic effect treats fatigue and/or intermittent dizziness and said administered product comprises at least one of CBD, THC, CBC, CBG, CBN, CBL, THCV and CBDV. According to an embodiment said therapeutic effect treats fatigue and/or intermittent dizziness and said product comprises CBD and optionally THC at CBD to THC weight/weight ratio greater than 1, greater than 2, greater than 3, greater than 4 or greater than 5. According to an embodiment said therapeutic effect treats fatigue and/or intermittent dizziness and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats fatigue and/or intermittent dizziness and said the primary terpene is selected from the group consisting of pinene, limonene, terpinene, eucalyptol, myrcene, terpineol, linalool, caryophyllene and combinations thereof. According to an embodiment, the product comprises at least 2 of these terpenes, at least 3, at least 4, or at least 5 of the terpenes. According to an embodiment, the primary terpene is selected from the group consisting of pinene, limonene, eucalyptol and combinations thereof. According to various embodiments said primary terpene comprises terpinene, myrcene and/or caryophyllene. According to an embodiment, said therapeutic effect treats fatigue and/or intermittent dizziness and the product further comprises an herbal extract selected from the group consisting of eleutherococcus senticosus, ganoderma lucidum, grifola frondosa, lentinula edodes, lepidium meyenii, panax ginseng, rhodiola rosea and combinations thereof.

According to an embodiment said therapeutic effect treats menopausal skin, itchy skin, age-related wrinkles and/or stretch marks and said administered product comprises at least one of CBD, THC, CBC, CBG, CBN, CBL, THCV and CBDV. According to an embodiment said therapeutic effect treats menopausal skin, itchy skin, age-related wrinkles and/or stretch marks and said product comprises CBD and optionally THC at CBD to THC weight/weight ratio greater than 1, greater than 2, greater than 3, greater than 4 or greater than 5. According to an embodiment said therapeutic effect treats menopausal skin, itchy skin, age-related wrinkles and/or stretch marks and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats menopausal skin, itchy skin, age-related wrinkles and/or stretch marks and said the primary terpene is selected from the group consisting of linalool limonene, nerolidol, pinene terpineol, myrcene, geraniol, nerol, citronellol, farnesol, carotol and combinations thereof. According to an embodiment, the product comprises at least 2 of these terpenes, at least 3, at least 4, or at least 5 of the terpenes. According to an embodiment, the primary terpene is selected from the group consisting of linalool, terpineol, geraniol and combinations thereof. According to various embodiments said primary terpene comprises limonene, nerolidol, pinene myrcene, nerol, citronellol, farnesol and/or carotol. According to an embodiment, said therapeutic effect treats menopausal skin, itchy skin, age-related wrinkles and/or stretch marks and the product further comprises a compound selected from the group consisting of linalyl acetate, citronellyl formate, eugenol, benzy acetate, benzyl alcohol, biochanin A, formonenetin, genistein, daidzein, glycitein, prenylnaringenin, diosgenin, acetin, cimicifugoside and combinations thereof. According to an embodiment, said therapeutic effect treats menopausal skin, itchy skin, age-related wrinkles and/or stretch marks and the product further comprises an herbal extract selected from the group consisting of glycyrrhiza glabra, plantago spp, symphytum officinalis, trifolium pratense, trigonella foenum graecum, ulmus rubra\fulva, verbascum thapsus and combinations thereof.

According to an embodiment said therapeutic effect treats headache and/or migraine and said administered product comprises at least one of CBD, THC, CBC, CBG, CBN, CBL, THCV and CBDV. According to an embodiment said therapeutic effect treats headache and/or migraine and said product comprises CBD and optionally THC at CBD to THC weight/weight ratio greater than 1, greater than 2, greater than 3, greater than 4 or greater than 5. According to an embodiment said therapeutic effect treats headache and/or migraine and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats headache and/or migraine and said the primary terpene is selected from the group consisting of myrcene, linalool, caryophyllene, eucalyptol, limonene, nerolidol, fenchol, humulene, guaiol, borneol, pinene, bisabolol, terpinene, terpineol and combinations thereof. According to an embodiment, the product comprises at least 2 of these terpenes, at least 3, at least 4, or at least 5 of the terpenes. According to an embodiment, the primary terpene is selected from the group consisting of myrcene, linalool, caryophyllene and combinations thereof. According to various embodiments said primary terpene comprises eucalyptol, limonene, nerolidol, fenchol, humulene, guaiol, linalool, limonene, borneol, pinene and/or bisabolol. According to an embodiment, said therapeutic effect treats headache and/or migraine and the product further comprises an herbal extract selected from the group consisting of angelica sinensis, corydalis yanhusuo, piscidia erythrina, rosmarinus officinalis, stachys betonica, tanacetum parthenium, verbena officinalis and combinations thereof. According to an embodiment, the product includes bisabolol, terpinene, humulene and/or linalool wherein bisabolol, terpinene, humulene and/or linalool forms less than 5% by weight of the total terpene content.

According to an embodiment said therapeutic effect treats weight gain and said product administered comprises at least one of CBD, THC, CBC, CBG, CBN, CBL, THCV and CBDV. According to an embodiment said therapeutic effect treats weight gain and said product comprises CBD and optionally THC at CBD to THC weight/weight ratio greater than 1, greater than 2, greater than 3, greater than 4 or greater than 5. According to an embodiment said therapeutic effect treats weight gain and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats weight gain and said the primary terpene is selected from the group consisting of eucalyptol, caryophyllene, limonene, humulene, phytol and combinations thereof. According to an embodiment, the product comprises at least 2 of these terpenes, at least 3, at least 4, or at least 5 of the terpenes. According to an embodiment, the primary terpene is selected from the group consisting of eucalyptol, limonene, humulene and combinations thereof. According to various embodiments said primary terpene comprises caryophyllene and/or phytol. According to an embodiment, said therapeutic effect treats weight gain and the product further comprises an herbal extract selected from the group consisting of ephedra sinica, foeniculum vulgare, fucus vesiculosus, garcinia cambogia, gymnema sylvestre, laminaria spp, paullinia cupana and combinations thereof.

According to an embodiment said therapeutic effect treats digestive problems, intestinal disorders, abdominal pain (colic), nausea, constipation and/or diarrhea and said administered product comprises at least one of CBD, THC, CBC, CBG, CBN, CBL, THCV and CBDV. According to an embodiment said therapeutic effect treats digestive problems, intestinal disorders, abdominal pain (colic), nausea, constipation and/or diarrhea and said product comprises CBD and optionally THC at CBD to THC weight/weight ratio greater than 1, greater than 2, greater than 3, greater than 4 or greater than 5. According to an embodiment said therapeutic effect treats digestive problems, intestinal disorders, abdominal pain (colic), nausea, constipation and/or diarrhea and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats digestive problems, intestinal disorders, abdominal pain (colic), nausea, constipation and/or diarrhea and said the primary terpene is selected from the group consisting of limonene, cymene, terpinene, caryophyllene, citronellol, sabinene, myrcene, linalool, germacrene, geraniol, pinene, terpineol and combinations thereof. According to an embodiment, the product comprises at least 2 of these terpenes, at least 3, at least 4, or at least 5 of the terpenes. According to an embodiment, the primary terpene is selected from the group consisting of limonene, sabinene, myrcene and combinations thereof. According to various embodiments said primary terpene comprises cymene, terpinene, caryophyllene, citronellol, linalool, germacrene, geraniol, terpineol and/or pinene. According to an embodiment, said therapeutic effect treats digestive problems, intestinal disorders, abdominal pain (colic), nausea, constipation and/or diarrhea and the product further comprises piperine. According to an embodiment, the product includes pinene, bisabolol, limonene and/or terpineol, wherein pinene, bisabolol, limonene and/or terpineol forms less than 5% by weight of the total terpene content

According to an embodiment said therapeutic effect treats breast pain and/or Mastalgia and said administered product comprises at least one of CBD, THC, CBC, CBG, CBN, CBL, THCV and CBDV. According to an embodiment said therapeutic effect treats breast pain and/or Mastalgia and said product comprises CBD and optionally THC at CBD to THC weight/weight ratio greater than 1, greater than 2, greater than 3, greater than 4 or greater than 5. According to an embodiment said therapeutic effect treats breast pain and/or Mastalgia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats pain and/or Mastalgia and said the primary terpene is selected from the group consisting of pinene, limonene, caryophyllene, myrcene, linalool, geraniol, eucalyptol, terpineol, sabinene and combinations thereof. According to an embodiment, the product comprises at least 2 of these terpenes, at least 3, at least 4, or at least 5 of the terpenes. According to an embodiment, the primary terpene is selected from the group consisting of pinene, linalool, eucalyptol and combinations thereof. According to various embodiments said primary terpene comprises limonene, caryophyllene, myrcene, geraniol, terpineol and/or sabinene. According to an embodiment, said therapeutic effect treats pain and/or Mastalgia and the product further comprises a compound selected from the group consisting of biochanin A, formonenetin, genistein, daidzein, glycitein, prenylnaringenin, diosgenin, acetin, cimicifugoside and combinations thereof.

According to an embodiment said therapeutic effect treats dysmenorrhea, pelvic pain, spasms, cramps, labor contraction and/or endometriosis and said administered product comprises at least one of CBD, THC, CBC, CBG, CBN, CBL, THCV and CBDV. According to an embodiment said therapeutic effect treats dysmenorrhea, pelvic pain, spasms, cramps, labor contraction and/or endometriosis and said product comprises CBD and optionally THC at CBD to THC weight/weight ratio greater than 1, greater than 2, greater than 3, greater than 4 or greater than 5. According to an embodiment said therapeutic effect treats dysmenorrhea, pelvic pain, spasms, cramps, labor contraction and/or endometriosis and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats dysmenorrhea, pelvic pain, spasms, cramps, labor contraction and/or endometriosis and said the primary terpene is selected from the group consisting of myrcene, linalool, caryophyllene, eucalyptol, nerolidol, limonene, nerolidol, sabinene, humulene, terpineol, citronellol, geraniol, pinene, limonene, camphene, terpinene, borneol, camphor, menthone, iso-menthone, carvacrol, thymol, carene, nerol, farnesol, menth-2-en-1-ol and combinations thereof. According to an embodiment, the product comprises at least 2 of these terpenes, at least 3, at least 4, or at least 5 of the terpenes. According to an embodiment, the primary terpene is selected from the group consisting of linalool, eucalyptol, nerolidol and combinations thereof. According to an embodiment, the primary terpene is selected from the group consisting of linalool, myrcene, nerolidol and combinations thereof. According to various embodiments said primary terpene comprises myrcene, caryophyllene, limonene, nerolidol, sabinene, humulene, terpineol, citronellol, geraniol, pinene, limonene, camphene, terpinene, borneol, camphor, menthone, iso-menthone, carvacrol, thymol, carene, nerol, farnesol and/or menth-2-en-ol. According to an embodiment, said therapeutic effect treats dysmenorrhea, pelvic pain, spasms, cramps, labor contraction and/or endometriosis and the product further comprises a compound selected from the group consisting of linalyl acetate, geranyl acetate, eugenol, methylis-oeugenol, menthol, eugenyl acetate, piperine, chamazulene, citronllyl formate, benzyl acetate, benzyl alcohol, biochanin A, formonenetin, genistein, daidzein, glycitein, prenylnaringenin, diosgenin, acetin, cimicifugoside and combinations thereof. According to an embodiment, said therapeutic effect treats dysmenorrhea, pelvic pain, spasms, cramps, labor contraction and/or endometriosis and the product further comprises an herbal extract selected from the group consisting of cimicifuga (actaea) racemosa, paeonia lactiflora, viburnum opulus, viburnum opulus, vitex agnus castus and combinations thereof.

According to an embodiment said therapeutic effect treats arthralgia and/or arthritis and said administered product comprises at least one of CBD, THC, CBC, CBG, CBN, CBL, THCV and CBDV. According to an embodiment said therapeutic effect treats arthralgia and/or arthritis and said product comprises CBD and optionally THC at CBD to THC weight/weight ratio greater than 1, greater than 2, greater than 3, greater than 4 or greater than 5. According to an embodiment said therapeutic effect treats arthralgia and/or arthritis and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats arthralgia and/or arthritis and said the primary terpene is selected from the group consisting of myrcene, linalool, guaiol, caryophyllene, eucalyptol, pinene, nerolidol, sabinene, limonene, terpinene, menth-2-en-1-ol, geraniol, guaiol, humulene, bisabolol, borneol, fenchol, terpineol and combinations thereof. According to an embodiment, the product comprises at least 2 of these terpenes, at least 3, at least 4, or at least 5 of the terpenes. According to an embodiment, the primary terpene is selected from the group consisting of myrcene, pinene, sabinene and combinations thereof. According to an embodiment, the primary terpene is selected from the group consisting of terpineol, linalool, sabinene and combinations thereof. According to various embodiments said primary terpene comprises linalool, guaiol, caryophyllene, eucalyptol, nerolidol, limonene, terpinene, menth-2-en-1-ol, geraniol, guaiol, humulene, bisabolol, borneol, fenchol and/or terpineol. According to an embodiment, said therapeutic effect treats arthralgia and/or arthritis and the product further comprises a compound selected from the group consisting of linalyl acetate, geranyl acetate, eugenol, methylis-oeugenol, menthol, eugenyl acetate, piperine and combinations thereof. According to an embodiment, said therapeutic effect treats arthralgia and/or arthritis and the product further comprises an herbal extract selected from the group consisting of boswellia spp, capsicum spp, corydalis yanhusuo, harpagophytum procumbens, piscidia erythrina, salix alba, zingiber officinalis and combinations thereof. according to an embodiment, the product optionally includes humulene, pinene, bisabolol, borneol, fenchol, guaiol, linalool, ocimene, terpineol, terpinene and/or caryophyllene, wherein humulene, pinene, bisabolol, borneol, fenchol, guaiol, linalool, ocimene, terpineol, terpinene and/or caryophyllene forms less than 5% by weight of the total terpene content.

According to an embodiment said therapeutic effect treats hot flashes, facial flushing, night sweat, irritability, palpitation and/or tachycardia and said administered product comprises at least one of CBD, THC, CBC, CBG, CBN, CBL, THCV and CBDV. According to an embodiment said therapeutic effect treats hot flashes, facial flushing, night sweat, irritability, palpitation and/or tachycardia and said product comprises CBD and optionally THC at CBD to THC weight/weight ratio greater than 1, greater than 2, greater than 3, greater than 4 or greater than 5. According to an embodiment said therapeutic effect treats hot flashes, facial flushing, night sweat, irritability, palpitation and/or tachycardia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats hot flashes, facial flushing, night sweat, irritability, palpitation and/or tachycardia and said the primary terpene is selected from the group consisting of caryophyllene, myrcene, humulene, linalool, terpineol, geraniol, pinene, carene, citronellol, limonene, eucalyptol, sabinene, myrtenol, terpinene, champhene, borneol and combinations thereof. According to an embodiment, the product comprises at least 2 of these terpenes, at least 3, at least 4, or at least 5 of the terpenes. According to an embodiment, the primary terpene is selected from the group consisting of linalool, pinene, eucalyptol and combinations thereof. According to an embodiment, the primary terpene is selected from the group consisting of linalool, geraniol, limonene, eucalyptol and combinations thereof. According to various embodiments said primary terpene comprises caryophyllene, myrcene, humulene, terpineol, geraniol, pinene, carene, citronellol, limonene, sabinene, myrtenol, champhene, borneol and/or terpinene. According to an embodiment, said therapeutic effect treats hot flashes, facial flushing, night sweat, irritability, palpitation and/or tachycardia and the product further comprises a compound selected from the group consisting of linalyl acetate, geranyl acetate, citronellyl formate, biochanin A, formonenetin, genistein, daidzein, glycitein, prenylnaringenin, diosgenin, acetin, cimicifugoside and combinations thereof. According to an embodiment, said therapeutic effect treats hot flashes, facial flushing, night sweat, irritability, palpitation and/or tachycardia and the product further comprises an herbal extract selected from the group consisting of angelica sinensis, anemarrhenae asphodeloides, cimicifuga (actaea) racemosa, dioscorea villosa, salvia officinalis, salvia officinalis, rehmania glutinosa preparata and combinations thereof.

According to an embodiment said therapeutic effect treats inflammation, oxidative stress and/or paresthesia and said administered product comprises at least one of CBD, THC, CBC, CBG, CBN, CBL, THCV and CBDV. According to an embodiment said therapeutic effect treats inflammation, oxidative stress and/or paresthesia and said product comprises CBD and optionally THC at CBD to THC weight/weight ratio greater than 1, greater than 2, greater than 3, greater than 4 or greater than 5. According to an embodiment said therapeutic effect treats inflammation, oxidative stress and/or paresthesia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats inflammation, oxidative stress and/or paresthesia and said the primary terpene is selected from the group consisting of caryophyllene, pinene, myrcene, limonene, humulene, citronellol, eucalyptol, linalool, beta amyrin, cycloartenol, terpinene, terpineol, geraniol, carvacrol, thymol, cardinol, terpinene, menth-2-en-1-ol, carotol, humulene, nerolidol and combinations thereof. According to an embodiment, the product comprises at least 2 of these terpenes, at least 3, at least 4, or at least 5 of the terpenes. According to an embodiment, the primary terpene is selected from the group consisting of caryophyllene, pinene, eucalyptol and combinations thereof. According to various embodiments said primary terpene comprises myrcene, limonene, humulene, citronellol, linalool, beta amyrin, cycloartenol, terpinene, terpineol, geraniol, carvacrol, thymol, cardinol, terpinene, menth-2-en-1-ol, carotol, humulene and/or nerolidol. According to an embodiment, said therapeutic effect treats inflammation, oxidative stress and/or paresthesia and the product further comprises a compound selected from the group consisting of linalyl acetate, geranyl acetate, eugenol, eugenyl acetate, piperine and combinations thereof.

According to an embodiment said therapeutic effect treats cardiovascular disease, palpitation and tachycardia and said administered product comprises at least one of CBD, THC, CBC, CBG, CBN, CBL, THCV and CBDV. According to an embodiment said therapeutic effect treats cardiovascular disease, palpitation and tachycardia and said product comprises CBD and optionally THC at CBD to THC weight/weight ratio greater than 1, greater than 2, greater than 3, greater than 4 or greater than 5. According to an embodiment said therapeutic effect treats cardiovascular disease, palpitation and tachycardia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats cardiovascular disease, palpitation and tachycardia and said the primary terpene is selected from the group consisting of linalool, camphene, limonene, pinene, borneol and combinations thereof. According to an embodiment, the product comprises at least 2 of these terpenes, at least 3, at least 4, or all 5 of the terpenes. According to an embodiment, the primary terpene is selected from the group consisting of linalool, limonene, pinene and combinations thereof. According to various embodiments said primary terpene comprises camphene and/or borneol. According to an embodiment, said therapeutic effect treats cardiovascular disease, palpitation and tachycardia and the product further comprises an herbal extract selected from the group consisting of cimicifuga (actaea) racemosa, crataegus spp, leonurus spp, passiflora incarnata and combinations thereof.

According to an embodiment said therapeutic effect treats urine incontinence and/or lower urinary tract disorders and said administered product comprises at least one of CBD, THC, CBC, CBG, CBN, CBL, THCV and CBDV. According to an embodiment said therapeutic effect treats urine incontinence and/or lower urinary tract disorders and said product comprises CBD and optionally THC at CBD to THC weight/weight ratio greater than 1, greater than 2, greater than 3, greater than 4 or greater than 5. According to an embodiment said therapeutic effect treats urine incontinence and/or lower urinary tract disorders and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats urine incontinence and/or lower urinary tract disorders and said the primary terpene is selected from the group consisting of linalool, terpineol, pinene, carene, geraniol, thujone, camphor, viridiflorol, caryophyllene, germacrene, cirtonellol, eucalyptol, myrtenol, sanatalol, sabinene, limonene and combinations thereof. According to an embodiment, the product comprises at least 2 of these terpenes, at least 3, at least 4, or at least 5 of the terpenes. According to an embodiment, the primary terpene is selected from the group consisting of linalool, geraniol, pinene and combinations thereof. According to various embodiments said primary terpene comprises terpineol, carene, thujone, camphor, viridiflorol, caryophyllene, germacrene, cirtonellol, eucalyptol, myrtenol, sanatalol, sabinene and/or limonene According to an embodiment, said therapeutic effect treats urine incontinence and/or lower urinary tract disorders and the product further comprises a compound selected from the group consisting of linalyl acetate, geranyl acetate, citronellyl formate and combinations thereof. According to an embodiment, said therapeutic effect treats urine incontinence and/or lower urinary tract disorders and the product further comprises an herbal extract selected from the group consisting of barosma betulina, equisitum arvense, plantago spp, urtica dioicanettle and combinations thereof.

According to an embodiment said therapeutic effect treats vaginal dryness and said administered product comprises at least one of CBD, THC, CBC, CBG, CBN, CBL, THCV and CBDV. According to an embodiment said therapeutic effect treats vaginal dryness and said product comprises CBD and optionally THC at CBD to THC weight/weight ratio greater than 1, greater than 2, greater than 3, greater than 4 or greater than 5. According to an embodiment said therapeutic effect treats vaginal dryness and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV.

According to an embodiment said therapeutic effect treats vaginal dryness and said the primary terpene is selected from the group consisting of myrcene, humulene, caryophyllene, linalool, terpineol, geraniol, pinene, germacrene, cirtonellol, limonene, myrcene, terpinene, menth-2-en-1-ol, eucalyptol, myrtenol and combinations thereof. According to an embodiment, the product comprises at least 2 of these terpenes, at least 3, at least 4, or at least 5 of the terpenes. According to an embodiment, the primary terpene is selected from the group consisting of linalool, geraniol, pinene and combinations thereof. According to various embodiments said primary terpene myrcene, humulene, caryophyllene, terpineol, germacrene, cirtonellol, limonene, myrcene, terpinene, menth-2-en-1-ol, eucalyptol and/or myrtenol: According to an embodiment, said therapeutic effect treats vaginal dryness and the product further comprises a compound selected from the group consisting of linalyl acetate, geranyl acetate, citronellyl formate, biochanin A, formonenetin, genistein, daidzein, glycitein, prenylnaringenin, diosgenin, acetin, cimicifugoside and combinations thereof. According to an embodiment, said therapeutic effect treats vaginal dryness and the product further comprises an herbal extract selected from the group consisting of dioscorea villosa spp, vitex agnus castus, withania somnifera, schisandra chinensis and combinations thereof.

According to an embodiment said therapeutic effect treats female urogenital and/or reproductive system infection and said administered product comprises at least one of CBD, THC, CBC, CBG, CBN, CBL, THCV and CBDV. According to an embodiment said therapeutic effect treats female urogenital and/or reproductive system infection and said product comprises CBD and optionally THC at CBD to THC weight/weight ratio greater than 1, greater than 2, greater than 3, greater than 4 or greater than 5. According to an embodiment said therapeutic effect treats female urogenital and/or reproductive system infection and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats female urogenital and/or reproductive system infection and said the primary terpene is selected from the group consisting of pinene, limonene, cymene, caryophyllene oxide, ocimene, nerolidol, eucalyptol, terpinene, terpineol, camphor, linalool, sabinene, thujone, camphor, viridiflorol, menthol, iso-menthone, caryophyllene, germacrene, geraniol, nerol, neral, geranial, citronellol, carvacol, thymol, carene, myrtenol, carene, myrcene, menth-2-en-1-ol and combinations thereof. According to an embodiment, the product comprises at least 2 of these terpenes, at least 3, at least 4, or at least 5 of the terpenes. According to an embodiment, the primary terpene is selected from the group consisting of caryophyllene oxide, pinene, limonene, geraniol and combinations thereof. According to an embodiment, the primary terpene is selected from the group consisting of caryophyllene oxide, pinene, limonene and combinations thereof. According to various embodiments said primary terpene comprises cymene, ocimene, nerolidol, eucalyptol, terpinene, terpineol, camphor, linalool, sabinene, thujone, camphor, viridiflorol, menthol, iso-menthone, caryophyllene, germacrene, geraniol, nerol, neral, geranial, citronellol, carvacol, thymol, carene, myrtenol, carene, myrcene and/or menth-2-en-1-ol. According to an embodiment, said therapeutic effect treats female urogenital and/or reproductive system infection and the product further comprises a compound selected from the group consisting of linalyl acetate, geranyl acetate, eugenol, eugenyl acetate, piperine, menthol, citronellyl formate and combinations thereof. According to an embodiment, said therapeutic effect treats female urogenital and/or reproductive system infection and the product further comprises an herbal extract selected from the group consisting of anemarrhenae asphodeloides, cimicifuga (actaea) racemosa, coptis chinensis, echinacea spp, hydrastis canadensis and combinations thereof. according to an embodiment the therapeutic effect treats human papillomavirus (hpv).

According to an embodiment said therapeutic effect treats fibroid, myomas and/or cysts in the female genital system and said administered product comprises at least one of CBD, THC, CBC, CBG, CBN, CBL, THCV and CBDV. According to an embodiment said therapeutic effect treats fibroid, myomas and/or cysts in the female genital system and said product comprises CBD and optionally THC at CBD to THC weight/weight ratio greater than 1, greater than 2, greater than 3, greater than 4 or greater than 5. According to an embodiment said therapeutic effect treats fibroid, myomas and/or cysts in the female genital system and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats fibroid, myomas and/or cysts in the female genital system and said the primary terpene is selected from the group consisting of linalool, terpineol, limonene, pinene, caryophyllene, caryophyllene oxide, terpinene, citronellol and combinations thereof. According to an embodiment, the product comprises at least 2 of these terpenes, at least 3, at least 4, or at least 5 of the terpenes. According to an embodiment, the primary terpene is selected from the group consisting of limonene, pinene, terpinene and combinations thereof. According to various embodiments said primary terpene comprises linalool, terpineol, caryophyllene, caryophyllene oxide and/or citronellol. According to an embodiment, said therapeutic effect treats fibroid, myomas and/or cysts in the female genital system and the product further comprises an herbal extract selected from the group consisting of angellica sinesis, larrea mexicana, viburnum prunifolium, vitex agnus castus, trifolium and combinations thereof.

According to an embodiment said therapeutic effect treats cystitis, urethritis and/or pyelonephritis and said administered product comprises at least one of CBD, THC, CBC, CBG, CBN, CBL, THCV and CBDV. According to an embodiment said therapeutic effect treats cystitis, urethritis and/or pyelonephritis and said product comprises CBD and optionally THC at CBD to THC weight/weight ratio greater than 1, greater than 2, greater than 3, greater than 4 or greater than 5. According to an embodiment said therapeutic effect treats cystitis, urethritis and/or pyelonephritis and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats cystitis, urethritis and/or pyelonephritis and said the primary terpene is selected from the group consisting of thujone, pinene, limonene, cymene, caryophyllene, caryophyllene oxide, eucalyptol, terpineol, sabinene, linalool, cardinol and combinations thereof. According to an embodiment, the product comprises at least 2 of these terpenes, at least 3, at least 4, or at least 5 of the terpenes. According to an embodiment, the primary terpene is selected from the group consisting of limonene, eucalyptol, linalool and combinations thereof. According to an embodiment, the primary terpene is selected from the group consisting of limonene, pinene, linalool and combinations thereof. According to various embodiments said primary terpene comprises thujone, pinene, cymene, caryophyllene, caryophyllene oxide, terpineol, sabinene and/or cardinol. According to an embodiment, said therapeutic effect treats cystitis, urethritis and/or pyelonephritis and the product further comprises an herbal extract selected from the group consisting of Althea officinalis, andrographis paniculata, anemarrhenae asphodeloides, apium graveolens, arctostaphylos uva-ursi, barosma betulina, coptis chinensis, echinacea spp, hydrastis canadensis, plantago spp, uncaria tomentosa, zea mays and combinations thereof.

According to an embodiment said therapeutic effect treats libido and/or sexual disfunction and said administered product comprises at least one of CBD, THC, CBC, CBG, CBN, CBL, THCV and CBDV. According to an embodiment said therapeutic effect treats libido and/or sexual disfunction and said product comprises CBD and optionally THC at CBD to THC weight/weight ratio greater than 1, greater than 2, greater than 3, greater than 4 or greater than 5. According to an embodiment said therapeutic effect treats libido and/or sexual disfunction and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats libido and/or sexual disfunction and said the primary terpene is selected from the group consisting of limonene, linalool, terpineol, geraniol, eucalyptol, pinene, citronellol and combinations thereof. According to an embodiment, the product comprises at least 2 of these terpenes, at least 3, at least 4, or at least 5 of the terpenes. According to an embodiment, the primary terpene is selected from the group consisting of limonene, linalool, ecualyptol and combinations thereof. According to various embodiments said primary terpene comprises terpineol, geraniol and/or pinene: According to an embodiment, said therapeutic effect treats libido and/or sexual disfunction and the product further comprises a compound selected from the group consisting of biochanin A, formonenetin, genistein, daidzein, glycitein, prenylnaringenin, diosgenin, acetin, cimicifugoside and combinations thereof. According to an embodiment, said therapeutic effect treats libido and/or sexual disfunction and the product further comprises an herbal extract selected from the group consisting of epimedium grandiflorum, ganoderma lucidum, lepidium meyenii, turnera diffusa, vitex agnus castus, withania somnifera and combinations thereof.

According to an embodiment said therapeutic effect treats hair loss and/or thinning and said administered product comprises at least one of CBD, THC, CBC, CBG, CBN, CBL, THCV and CBDV. According to an embodiment said therapeutic effect treats hair loss and/or thinning and said product comprises CBD and optionally THC at CBD to THC weight/weight ratio greater than 1, greater than 2, greater than 3, greater than 4 or greater than 5. According to an embodiment said therapeutic effect treats hair loss and/or thinning and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats hair loss and/or thinning and said the primary terpene is selected from the group consisting of linalool, terpineol, eucalyptol, borneol, camphor, pinene, geraniol and combinations thereof. According to an embodiment, the product comprises at least 2 of these terpenes, at least 3, at least 4, or at least 5 of the terpenes. According to an embodiment, the primary terpene is selected from the group consisting of geraniol, eucalyptol, terpineol and combinations thereof. According to various embodiments said primary terpene comprises linalool, borneol, camphor and/or pinene According to an embodiment, said therapeutic effect treats hair loss and/or thinning and the product further comprises an herbal extract selected from the group consisting of rehmania glutinosa preparata, rosmarinus officinalis, serenoa serrulata, urtica dioica and combinations thereof.

According to an embodiment said therapeutic effect treats brittle nails and said administered product comprises at least one of CBD, THC, CBC, CBG, CBN, CBL, THCV and CBDV. According to an embodiment said therapeutic effect treats brittle nails and said product comprises CBD and optionally THC at CBD to THC weight/weight ratio greater than 1, greater than 2, greater than 3, greater than 4 or greater than 5. According to an embodiment said therapeutic effect treats brittle nails and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats brittle nails and said the primary terpene is selected from the group consisting of linalool, eucalyptol, geraniol, pinene, limonene and combinations thereof. According to an embodiment, the product comprises at least 2 of these terpenes, at least 3, at least 4, or all 5 of the terpenes. According to an embodiment, the primary terpene is selected from the group consisting of linalool, eucalyptol, limonene and combinations thereof. According to various embodiments said primary terpene comprises geraniol and/or pinene.

According to an embodiment said therapeutic effect treats irregular body odor and said administered product comprises at least one of CBD, THC, CBC, CBG, CBN, CBL, THCV and CBDV. According to an embodiment said therapeutic effect treats irregular body odor and said product comprises CBD and optionally THC at CBD to THC weight/weight ratio greater than 1, greater than 2, greater than 3, greater than 4 or greater than 5. According to an embodiment said therapeutic effect treats irregular body odor and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats irregular body odor and said the primary terpene is selected from the group consisting of linalool, terpineol, geraniol, eucalyptol and combinations thereof. According to an embodiment, the product comprises at least 2 of these terpenes, at least 3, or all 4 of the terpenes. According to an embodiment, the primary terpene is selected from the group consisting of linalool, terpineol, geraniol and combinations thereof.

According to an embodiment said therapeutic effect treats Polycystic Ovary Syndrome (POCS), amenorrhea, oligomenorrhea, hypermenorrhea, metrorrhagia and said administered product comprises at least one of CBD, THC, CBC, CBG, CBN, CBL, THCV and CBDV. According to an embodiment said therapeutic effect treats Polycystic Ovary Syndrome (POCS), amenorrhea, oligomenorrhea, hypermenorrhea, metrorrhagia and said product comprises CBD and optionally THC at CBD to THC weight/weight ratio greater than 1, greater than 2, greater than 3, greater than 4 or greater than 5. According to an embodiment said therapeutic effect treats Polycystic Ovary Syndrome (POCS), amenorrhea, oligomenorrhea, hypermenorrhea, metrorrhagia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats Polycystic Ovary Syndrome (POCS), amenorrhea, oligomenorrhea, hypermenorrhea, metrorrhagia and said the primary terpene is selected from the group consisting of humulene, linalool, terpineol, caryophyllene, caryophyllene oxide, terpinene, citronellol, geraniol, thujone, camphor, viridiflorol, pinene, camphene, sabinene, myrcene, eucalyptol, carene, carotol, limonene, sabinene, cardinal, pinene and combinations thereof. According to an embodiment, the product comprises at least 2 of these terpenes, at least 3, at least 4, or at least 5 of the terpenes. According to an embodiment, the primary terpene is selected from the group consisting of linalool, eucalyptol, geraniol and combinations thereof. According to an embodiment, the primary terpene is selected from the group consisting of linalool, eucalyptol, caryophyllene, pinene and combinations thereof. According to various embodiments said primary terpene comprises humulene, terpineol, caryophyllene, caryophyllene oxide, terpinene, citronellol, thujone, camphor, viridiflorol, pinene, camphene, sabinene, myrcene, carene, carotol, limonene, sabinene, cardinal and/or pinene. According to an embodiment, said therapeutic effect treats Polycystic Ovary Syndrome (POCS), amenorrhea, oligomenorrhea, hypermenorrhea, metrorrhagia and the product further comprises a compound selected from the group consisting of linalyl acetate, geranyl acetate, eugenol, chamazulene, citronellyl formate and combinations thereof. According to an embodiment, said therapeutic effect treats Polycystic Ovary Syndrome (POCS), amenorrhea, oligomenorrhea, hypermenorrhea, metrorrhagia and the product further comprises an herbal extract selected from the group consisting of angelica sinensis, cimicifuga (actaea) racemosa, dioscorea villosa spp., vitex agnus castus, humulus lupulus, hypericum perforatum, lavendula officinalis, melissa officinalis, passiflora incarnata, valeriana officinalis and combinations thereof.

According to an embodiment said therapeutic effect treats premenstrual syndrome (PMS), premenstrual tension, and/or premenstrual dysphoric disorder (PMDD) and said administered product comprises at least one of CBD, THC, CBC, CBG, CBN, CBL, THCV and CBDV. According to an embodiment said therapeutic effect treats premenstrual syndrome (PMS), premenstrual tension, and/or premenstrual dysphoric disorder (PMDD) and said product comprises CBD and optionally THC at CBD to THC weight/weight ratio greater than 1, greater than 2, greater than 3, greater than 4 or greater than 5. According to an embodiment said therapeutic effect treats premenstrual syndrome (PMS), premenstrual tension, and/or premenstrual dysphoric disorder (PMDD) and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats premenstrual syndrome (PMS), premenstrual tension, and/or premenstrual dysphoric disorder (PMDD) and said the primary terpene is selected from the group consisting of limonene, linalool, eucalyptol, nerolidol, sabinene, pinene, caryophyllene, terpineol, geraniol, thujone, camphor, viridiflorol, champhene, myrcene, caryophyllene, terpinene, carene, cirtonellol, carotol, neral, geranial and combinations thereof. According to an embodiment, the product comprises at least 2 of these terpenes, at least 3, at least 4, or at least 5 of the terpenes. According to an embodiment, the primary terpene is selected from the group consisting of linalool, eucalyptol, geraniol and combinations thereof. According to an embodiment, the primary terpene is selected from the group consisting of linalool, caryophyllene, limonene and combinations thereof. According to an embodiment, the primary terpene is selected from the group consisting of linalool, caryophyllene, limonene, eucalyptol, geraniol and combinations thereof. According to various embodiments said primary terpene comprises limonene, nerolidol, sabinene, pinene, caryophyllene, terpineol, thujone, camphor, viridiflorol, champhene, myrcene, caryophyllene, terpinene, carene, cirtonellol, carotol, neral and/or geranial. According to an embodiment, said therapeutic effect treats premenstrual syndrome (PMS), premenstrual tension, and/or premenstrual dysphoric disorder (PMDD) and the product further comprises a compound selected from the group consisting of linalyl acetate, geranyl acetate, citronellyl formate, menthol, eugenol piperine, jasmone and combinations thereof. According to an embodiment, said therapeutic effect treats premenstrual syndrome (PMS), premenstrual tension, and/or premenstrual dysphoric disorder (PMDD) and the product further comprises an herbal extract selected from the group consisting angelica sinensis, cimicifuga (actaea) racemosa, dioscorea villosa spp, paeonia lactiflora, vitex agnus castus and combinations thereof.

According to an embodiment said therapeutic effect hormonal disfunction and/or imbalance and/or fertility and said administered product comprises at least one of CBD, THC, CBC, CBG, CBN, CBL, THCV and CBDV. According to an embodiment said therapeutic effect treats hormonal disfunction and/or imbalance and/or fertility and said product comprises CBD and optionally THC at CBD to THC weight/weight ratio greater than 1, greater than 2, greater than 3, greater than 4 or greater than 5. According to an embodiment said therapeutic effect treats hormonal disfunction and/or imbalance and/or fertility and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats hormonal disfunction and/or imbalance and/or fertility and said the primary terpene is selected from the group consisting of linalool, terpineol, geraniol, thujone, camphor, viridiflorol, borneol, champhene, pinene, eucalyptol, caryophyllene, germacrene, pinene, carene, cirtonellol, nerol, farnesol, limonene, myrcene, terpinene, menth-2-en-1-ol, carotol, sabinene, neral, geranial and combinations thereof. According to an embodiment, the product comprises at least 2 of these terpenes, at least 3, at least 4, or at least 5 of the terpenes. According to an embodiment, the primary terpene is selected from the group consisting of linalool, eucalyptol, geraniol and combinations thereof. According to an embodiment, the primary terpene is selected from the group consisting of linalool, eucalyptol, geraniol, terpineol and combinations thereof. According to an embodiment, the primary terpene is selected from the group consisting of linalool, eucalyptol, geraniol, pinene, caryophyllene and combinations thereof. According to various embodiments said primary terpene comprises terpineol, geraniol, thujone, camphor, viridiflorol, borneol, champhene, pinene, caryophyllene, germacrene, pinene, carene, cirtonellol, nerol, farnesol, limonene, myrcene, terpinene, menth-2-en-1-ol, carotol, sabinene, neral and/or geranial. According to an embodiment, said therapeutic effect treats hormonal disfunction and/or imbalance and/or fertility and the product further comprises a compound selected from the group consisting of linalyl acetate, geranyl acetate, chamazulene, citronellyl formate, biochanin A, formonenetin, genistein, daidzein, glycitein, prenylnaringenin, sclareol, diosgenin, acetin, cimicifugoside and combinations thereof. According to an embodiment, said therapeutic effect treats hormonal disfunction and/or imbalance and/or fertility and the product further comprises an herbal extract selected from the group consisting angelica sinensis, cimicifuga (actaea) racemosa, dioscorea villosa spp, trifolium pratense, vitex agnus castus and combinations thereof.

According to an embodiment said therapeutic effect treats pre-eclampsia toxemia and said administered product comprises at least one of CBD, THC, CBC, CBG, CBN, CBL, THCV and CBDV. According to an embodiment said therapeutic effect treats pre-eclampsia toxemia and said product comprises CBD and optionally THC at CBD to THC weight/weight ratio greater than 1, greater than 2, greater than 3, greater than 4 or greater than 5. According to an embodiment said therapeutic effect treats pre-eclampsia toxemia and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats pre-eclampsia toxemia and said the primary terpene is selected from the group consisting of limonene, neral, geranial, caryophyllene, linalool, myrcene, nerolidol, fenchol, humulene, guaiol, borneol, cymene, terpinene and combinations thereof. According to an embodiment, the product comprises at least 2 of these terpenes, at least 3, at least 4, or at least 5 of the terpenes. According to an embodiment, the primary terpene is selected from the group consisting of linalool, myrcene, limonene and combinations thereof. According to an embodiment, the primary terpene is selected from the group consisting of linalool, myrcene, limonene, caryophyllene and combinations thereof. According to various embodiments said primary terpene comprises neral, geranial, caryophyllene, nerolidol, fenchol, humulene, guaiol, borneol, cymene and/or terpinene. According to an embodiment, said therapeutic effect treats pre-eclampsia toxemia and the product further comprises an herbal extract selected from the group consisting of crataegus spp, olea europea, passiflora incarnata and combinations thereof.

According to an embodiment said therapeutic effect treats conditions and/or symptoms associated with female hormonal disfunction and/or imbalance and said administered product comprises at least one of CBD, THC, CBC, CBG, CBN, CBL, THCV and CBDV. According to an embodiment said therapeutic effect treats conditions and/or symptoms associated with female hormonal disfunction and/or imbalance and said product comprises CBD and optionally THC at CBD to THC weight/weight ratio greater than 1, greater than 2, greater than 3, greater than 4 or greater than 5. According to an embodiment said therapeutic effect treats conditions and/or symptoms associated with female hormonal disfunction and/or imbalance and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats conditions and/or symptoms associated with female hormonal disfunction and/or imbalance and said the primary terpene is selected from the group consisting of linalool, terpineol, geraniol, thujone, camphor, viridiflorol, borneol, champhene, pinene, eucalyptol, caryophyllene, germacrene, pinene, carene, cirtonellol, nerol, farnesol, limonene, myrcene, terpinene, menth-2-en-1-ol, carotol, sabinene, neral, geranial, nerolidol, terpineol, carene and combinations thereof. According to an embodiment, the product comprises at least 2 of these terpenes, at least 3, at least 4, or at least 5 of the terpenes. According to an embodiment, the primary terpene is selected from the group consisting of geraniol, linalool, eucalyptol and combinations thereof. According to an embodiment, the primary terpene is selected from the group consisting of terpineol, linalool, caryophyllene, pinene and combinations thereof. According to an embodiment, the primary terpene is selected from the group consisting of geraniol, linalool, eucalyptol, caryophyllene, terpineol, pinene, limonene and combinations thereof. According to various embodiments said primary terpene comprises terpineol, geraniol, thujone, camphor, viridiflorol, borneol, champhene, pinene, eucalyptol, caryophyllene, germacrene, pinene, carene, cirtonellol, nerol, farnesol, limonene, myrcene, terpinene, menth-2-en-1-ol, carotol, sabinene, neral, geranial, nerolidol, terpineol and/or carene. According to an embodiment, said therapeutic effect treats conditions and/or symptoms associated with female hormonal disfunction and/or imbalance and the product further comprises a compound selected from the group consisting of biochanin A, formonenetin, genistein, daidzein, glycitein, prenylnaringenin, diosgenin, acetin, cimicifugoside and combinations thereof. According to an embodiment, said therapeutic effect treats female hormonal disfunction and/or imbalance and the product further comprises an herbal extract selected from the group consisting of angelica sinensis, chamaelirium lutea, cimicifuga (actaea) racemosa, dioscorea villosa spp., trifolium pratense, vitex agnus castus and combinations thereof.

According to an embodiment said therapeutic effect treats conditions and/or symptoms associated with perimenopause and/or menopause and said administered product comprises at least one of CBD, THC, CBC, CBG, CBN, CBL, THCV and CBDV. According to an embodiment said therapeutic effect treats conditions and/or symptoms associated with perimenopause and/or menopause and said product comprises CBD and optionally THC at CBD to THC weight/weight ratio greater than 1, greater than 2, greater than 3, greater than 4 or greater than 5. According to an embodiment said therapeutic effect treats conditions and/or symptoms associated with perimenopause and/or menopause and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats conditions and/or symptoms associated with perimenopause and/or menopause and said the primary terpene is selected from the group consisting of caryophyllene, caryophyllene oxide, carene, linalool, myrcene, terpinene, terpineol, citronellol, eucalyptol, nerolidol, limonene, geraniol, menthol, iso-menthone, germacrene, pinene, cardinol, neral, geranial, menth-2-en-1-ol, pulegone, cymene, fenchol, humulene, guaiol, borneol, carene, sabinene, myrtenol, camphene, nerol, farnesol, carotol and combinations thereof. According to an embodiment, the product comprises at least 2 of these terpenes, at least 3, at least 4, or at least 5 of the terpenes. According to an embodiment, the primary terpene is selected from the group consisting of caryophyllene, linalool, carene and combinations thereof. According to an embodiment, the primary terpene is selected from the group consisting of caryophyllene, linalool, myrcene and combinations thereof. According to an embodiment, the primary terpene is selected from the group consisting of nerolidol, limonene, linalool and combinations thereof. According to an embodiment, the primary terpene is selected from the group consisting of caryophyllene, linalool, carene, nerolidol, limonene and combinations thereof. According to various embodiments said primary terpene comprises caryophyllene, caryophyllene oxide, carene, myrcene, terpinene, terpineol, citronellol, eucalyptol, nerolidol, limonene, geraniol, menthol, iso-menthone, germacrene, pinene, cardinol, neral, geranial, menth-2-en-1-ol, pulegone, cymene, fenchol, humulene, guaiol, borneol, carene, sabinene, myrtenol, camphene, nerol, farnesol and/or carotol. According to an embodiment, said therapeutic effect treats conditions and/or symptoms associated with perimenopause and/or menopause and the product further comprises a compound selected from the group consisting of biochanin A, formonenetin, genistein, daidzein, glycitein, prenylnaringenin, diosgenin, acetin, cimicifugoside and combinations thereof. According to an embodiment, said therapeutic effect treats conditions and/or symptoms associated with perimenopause and/or menopause and the product further comprises an herbal extract selected from the group consisting of angelica sinensis, anemarrhenae asphodeloides, cimicifuga (actaea) racemosa, glycyrrhiza glabra. hypericum perforatum, panax quinquefolium, trifolium pratense, avena sativa, cimicifuga (actaea) racemosa, humulus lupulus, hypericum perforatum, lavendula officinalis, melissa officinalis, passiflora incarnata, valeriana officinalis and combinations thereof.

According to an embodiment said therapeutic effect treats pain and/or cramps and said administered product comprises at least one of CBD, THC, CBC, CBG, CBN, CBL, THCV and CBDV. According to an embodiment said therapeutic effect treats pain and/or cramps and said product comprises CBD and optionally THC at CBD to THC weight/weight ratio greater than 1, greater than 2, greater than 3, greater than 4 or greater than 5. According to an embodiment said therapeutic effect treats pain and/or cramps and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats pain and/or cramps and said the primary terpene is selected from the group consisting of myrcene, linalool, caryophyllene, eucalyptol, nerolidol, sabinene, humulene, terpineol, citronellol, geraniol, pinene, limonene, camphene, terpinene, borneol, camphor, menthone, iso-menthone, carvacrol, thymol, carene, nerol, farnesol, menth-2-en-1-ol, guaiol and combinations thereof. According to an embodiment, the product comprises at least 2 of these terpenes, at least 3, at least 4, or at least 5 of the terpenes. According to an embodiment, the primary terpene is selected from the group consisting of limonene, linalool, eucalyptol and combinations thereof. According to an embodiment, the primary terpene is selected from the group consisting of limonene, linalool, eucalyptol, myrcene and combinations thereof. According to various embodiments said primary terpene comprises myrcene, caryophyllene, nerolidol, sabinene, humulene, terpineol, citronellol, geraniol, pinene, camphene, terpinene, borneol, camphor, menthone, iso-menthone, carvacrol, thymol, carene, nerol, farnesol, menth-2-en-1-ol and/or guaiol. According to an embodiment, said therapeutic effect treats pain and/or cramps and the product further comprises an herbal extract selected from the group consisting of angelica sinensis, piscidia erythrina, tanacetum parthenium, viburnum opulus, boswellia spp, capsicum spp, corydalis yanhusuo, harpagophytum procumbens, rosmarinus officinalis, salix alba, zingiber officinalis and combinations thereof.

According to an embodiment said therapeutic effect treats female urogenital and/or reproductive system infections and/or disorders and said administered product comprises at least one of CBD, THC, CBC, CBG, CBN, CBL, THCV and CBDV. According to an embodiment said therapeutic effect treats female urogenital and/or reproductive system infections and/or disorders and said product comprises CBD and optionally THC at CBD to THC weight/weight ratio greater than 1, greater than 2, greater than 3, greater than 4 or greater than 5. According to an embodiment said therapeutic effect treats female urogenital and/or reproductive system infections and/or disorders and said product comprises THC and optionally CBD at THC to CBD weight/weight ratio greater than 0.9, greater than 2, greater than 3, greater than 4 or greater than 5. According to various embodiments, said product comprises CBD, comprises THC, comprises CBC, comprises CBG, comprises CBN, comprises CBL, comprises THCV and/or comprises CBDV. According to an embodiment said therapeutic effect treats female urogenital and/or reproductive system infections and/or disorders and said the primary terpene is selected from the group consisting of myrcene, humulene, caryophyllene, linalool, terpineol, geraniol, pinene, germacrene, cirtonellol, limonene, myrcene, terpinene, menth-2-en-1-ol, borneol, eucalyptol, myrtenol, cymene, caryophyllene oxide, ocimene, nerolidol, camphor, sabinene, thujone, camphor, viridiflorol, menthol, iso-menthone, caryophyllene, germacrene, geraniol, nerol, neral, geranial, citronellol, carvacol, thymol, carene, myrtenol, carene, thujone and combinations thereof. According to an embodiment, the product comprises at least 2 of these terpenes, at least 3, at least 4, or at least 5 of the terpenes. According to an embodiment, the primary terpene is selected from the group consisting of linalool, pinene, caryophyllene oxide and combinations thereof. According to an embodiment, the primary terpene is selected from the group consisting of geraniol, linalool, pinene, caryophyllene oxide and combinations thereof. According to various embodiments said primary terpene comprises myrcene, humulene, caryophyllene, terpineol, geraniol, germacrene, cirtonellol, limonene, myrcene, terpinene, menth-2-en-1-ol, borneol, eucalyptol, myrtenol, cymene, ocimene, nerolidol, camphor, sabinene, thujone, camphor, viridiflorol, menthol, iso-menthone, caryophyllene, germacrene, geraniol, nerol, neral, geranial, citronellol, carvacol, thymol, carene, myrtenol, carene and/or thujone. According to an embodiment, said therapeutic effect treats female urogenital and/or reproductive system infections and/or disorders and the product further comprises an herbal extract selected from the group consisting of Apium graveolens, dioscorea villosa spp, althea officinalis, anemarrhenae asphodeloides, anemarrhenae asphodeloides, apium graveolens, arctostaphylos uva-ursi, barosma betulina, coptis chinensis, echinacea spp, equisitum arvense, hydrastis canadensis, plantago spp, serenoa serrulata, uncaria tomentosa, urtica dioica, viola tricolor, zea mays and combinations thereof.

According to an embodiment, terpene-enriched cannabinoid product is produced by adding isolated terpenes to a cannabis extractproduct.

According to an embodiment, the method for treating a patient comprises (i) administering to the patient for a first period of time a first terpene-enriched cannabis product comprising a first cannabinoid at a first cannabinoid amount and a first primary terpene at a first primary terpene amount, followed by (ii) administering to the patient for a second period of time a second terpene-enriched cannabis product comprising the first cannabinoid amount and a second primary terpenes at a second primary terpene amount. According to an embodiment, the method further comprises administering to the patient for a third period of time the first terpene-enriched cannabis product comprising the first cannabinoid amount and a third primary terpene at a third primary terpene amount. According to an embodiment, the third primary terpene is identical to the first primary terpene.

According to an embodiment, the method for treating a patient comprises administering to said subject for a first period of time a first said product comprising a first cannabinoid at a first cannabinoid amount and a first primary terpene at a first primary terpene amount, followed by (i) administering to said subject for a second period of time said product comprising said first cannabinoid at said first cannabinoid amount or less and a second primary terpene at a second primary terpene amount; (ii) administering to said subject for a second period of time said product comprising said first cannabinoid at said first cannabinoid amount or less and a first primary terpene at an increased first primary terpene amount; (iii) administering to said subject for a second period of time said product comprising said first cannabinoid at a said first cannabinoid amountor less, first primary terpene at a first primary terpene amount and at least one secondary terpene; and/or (iv) administering to said subject for a second period of time said product comprising said first cannabinoid at a said first cannabinoid amountor less, first primary terpene at a first primary terpene amount and a second primary terpene.

According to another embodiment, the method for treating a patient comprises (i) administering to the patient a first terpene-enriched cannabis product comprising a first cannabinoid at a first cannabinoid amount and a first primary terpene at a first primary terpene amount and administering to the patient, at least 2 hours later a second terpene-enriched cannabis product comprising the first cannabinoid amount and a second primary terpenes at a second primary terpene amount. According to another embodiment, the first terpene-enriched cannabis product is administered for day time and the second terpene-enriched cannabis product is administered for night time.

According to another embodiment, the method for treating a patient comprises administering to the patient (i) a first terpene-enriched cannabis product comprising a first cannabinoid at a first cannabinoid amount and a first primary terpene at a first primary terpene amount and (ii) a second terpene-enriched cannabis product comprising the first cannabinoid and a second primary terpenes at a second primary terpene amount.

According to an embodiment, the method for treating a patient comprises (i) administering to the patient multiple cannabis products to find the best working one; (ii) mimicking the best working product by extracting cannabis plant material to form an extract and blending the extract with suitable terpenes, whereby a mimicking product is formed and (iii) administering to the patient the mimicking product. According to an embodiment, the method further comprises removing terpenes from the extract prior to the blending.

According to an embodiment, the method for treating a patient comprises administering to said patient a product selected from the group consisting of tablets, gel capsules, medical patches, topicals, creams, varnishes, sublingual oils, sprays, edibles, suppositories including tampons and/or rectal candles, cigarettes, vaporizer liquids, micro and nano-emulsions, preparations containing micro and nano-particle and combinations thereof, containing the administered product. According to an embodiment, the product comprising tablets containing the product. According to an embodiment, the product comprising gel capsules containing the product. According to an embodiment, the product comprising medical patches containing the product. According to an embodiment, the product comprising topicals containing the product. According to an embodiment, the product comprising creams containing the product. According to an embodiment, the product comprising varnishes containing the product. According to an embodiment, the product comprising sublingual oils containing the product. According to an embodiment, the product comprising edibles containing the product. According to an embodiment, the product comprising tampons containing the product. According to an embodiment, the product comprising rectal candles containing the product. According to an embodiment, the product comprising cigarettes containing the product. According to an embodiment, the product comprising vaporizer liquids containing the product.

According to an embodiment, further provided is a method for achieving, reaching and/or gaining a therapeutic effect in treating conditions and/or symptoms associated with perimenopause, menopause, female urogenital or reproductive system infections and/or disorders, menstrual cycle, hormonal deficiency and/or hormonal imbalance comprising administering to a subject in need a therapeutically effective amount of a composition comprising (i) a primary terpene in a specific amount; (ii) optionally at least three secondary terpenes; (iii) optionally at least one cannabinoid in a specific amount; and (iv) optionally at least one phytoestrogen. According to an embodiment, the composition comprises less than 20% by weight water. According to an embodiment, the conditions and/or symptoms are selected from the group consisting of hot flashes, facial flushing, night sweat, osteoporosis, fatigue, irritability, insomnia and/or sleep disorder, difficulty in concentration, depression, memory loss, brain fog, headache, migraine, anxiety, nervousness, restless, stress, mood swing, affect disorder, general discomfort, muscle tension, myalgia, intermittent dizziness, paresthesia, indigestion, digestive problems, intestinal disorders, abdominal pain, irritability, palpitations, tachycardia, nausea, constipation, diarrhea, arthralgia and/or arthritis, dysmenorrheal, pelvic pain, spasms, cramps, labor contraction, endometriosis Polycystic Ovary Syndrome (POCS), amenorrhea, oligomenorrhea, hypermenorrhea, metrorrhagia, premenstrual syndrome (PMS), premenstrual tension, premenstrual dysphoric disorder (PMDD), cold hands and feet, weight gain, mental awareness problems, sexual pain disorder, dyspareunia, breast pain and/or mastalgia, menopausal skin, inflammation, oxidative stress, cardiovascular disease, loss of pelvic muscle tone, urinary incontinence, lower urinary tract disorders, vaginal dryness, women urogenital and/or reproductive system infection, fibroid, myomas and/or cysts in the female genital system, human papillomavirus (HPV), Uterine Fibroid, cystitis, urethritis, pyelonephritis, women hormonal dysfunction and/or imbalance, irregular periods, pre-eclampsia toxemia, reduced libido, reduced sexual dysfunction, hair loss and/or thinning, age-related winkles, stretch marks, brittle nails, gam disorders, irregular body odor, conditions and/or symptoms induced by estrogen deficiency and combinations thereof.

According to an embodiment, the administered composition additionally contains an additive selected from the group consisting of antioxidants, emulsifiers and texturizers vegetable oils, plant extracts, honey, sucrose, glucose and fructose, pharmaceutical excipients and combinations thereof.

According to an embodiment, the primary terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thujone, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

According to an embodiment, the composition comprises a primary terpene and optionally at least three secondary terpenes, at least four or at least five. The term “terpene”, as used herein, refers to both terpenes and terpenoids. As used here, the term “primary terpene” refers to a terpene that forms at least 20% by weight of the total amount of terpenes in the composition, at least 30%, at least 40%, at least 50%, at least 60%, at least 70% or at least 80%. According to an embodiment, the composition comprises multiple (e.g. two or three) terpenes, each one of which forms at least 20% by weight of the total amount of terpenes in the composition and each one of these terpenes is considered a primary terpene. As used here, the term “secondary terpene” refers to a terpene that forms at least 10 parts per million (ppm) of the composition. According to an embodiment, the content of the primary terpene in the composition is at least 2 times greater than that of any secondary terpene, at least 4, at least 6, at least 8, at least 10, at least 15, at least 20, at least 25, or at least 30 times greater.

According to an embodiment, the composition comprises at least one of isoflavones, coumestans, prenylflavonoids, lignans and saponins. According to an embodiment, the composition comprises isoflavones. According to an embodiment, the composition comprises coumestans. According to an embodiment, the composition comprises prenylflavonoids. According to an embodiment, the composition comprises lignans. According to an embodiment, the composition comprises saponins. According to an embodiment, the composition comprises at least one of biochanin A, formonenetin, genistein, daidzein, glycitein, prenylnaringenin, diosgenin, acetin and/or cimicifugoside. According to an embodiment, the composition comprises biochanin A. According to an embodiment, the composition comprises formonenetin. According to an embodiment, the composition comprises genistein. According to an embodiment, the composition comprises daidzein. According to an embodiment, the composition comprises glycitein. According to an embodiment, the composition comprises prenylnaringenin. According to an embodiment, the composition comprises diosgenin. According to an embodiment, the composition comprises acetin. According to an embodiment, the composition comprises cimicifugoside.

According to an embodiment, the composition comprises at least 5% by weight carrier, at least 10%, at least 15%, at least 20%, at least 25%, at least 30%, at least 35% or at least 40% by weight. Any compound other than cannabinoids and terpenes is a suitable carrier. According to an embodiment, the carrier is selected from the group consisting of vegetable oils, e.g. coconut oil, olive oil or sesame oil, pharmaceutical excipients, honey, bees wax, cellulose and combinations thereof. According to an embodiment, the carrier is an essential oil. According to an embodiment, the carrier is an herbal extract.

According to an embodiment, the primary terpene is present in specific amounts, and the onset time of the therapeutic effect, as measured by methods known in the art, is at least 20% shorter than that of a composition comprising one half the amount of the primary terpene, at least 30%, at least 40%, at least 50%, at least 60%, or at least 70% shorter. According to an embodiment, the primary terpene and secondary terpenes are present in specific amounts, and the onset time of the therapeutic effect is at least 20% shorter than that of a composition comprising the same secondary terpene amounts and one half the amount of the primary terpene, at least 30%, at least 40%, at least 50%, at least 60%, or at least 70% short. Shorter onset time, or differently put an earlier effect, is important particularly in cases of sublingual and topical delivery and in cases where a rapid effect is desired, as in treating pain.

According to an embodiment, the primary terpene is present in specific amounts, and the onset time of the therapeutic effect is at least 20% longer than that of a composition comprising one half the amount of the primary terpene, at least 30%, at least 40%, at least 50%, at least 60%, or at least 70% longer. According to an embodiment, the primary terpene and secondary terpenes are present in specific amounts, and the onset time of the therapeutic effect is at least 20% longer than that of a composition comprising the same secondary terpene amounts and one half the amount of the primary terpene, at least 30%, at least 40%, at least 50%, at least 60%, or at least 70% longer. According to an embodiment, compositions of delayed onset time are used in combination with shorter onset time to reach a sustained release effect.

According to an embodiment, the primary terpene is present in specific amounts, and the magnitude of the therapeutic effect, as measured by methods known in the art, is at least 20% greater compared with that of a composition comprising one half the amount of the primary terpene, at least 30%, at least 40%, at least 50%, at least 60%, or at least 70% greater. According to an embodiment, the primary terpene, and secondary terpenes are present in specific amounts, and the magnitude of the therapeutic effect is at least 20% greater than that of a composition comprising the same secondary terpene amounts and one half the amount of the primary terpene, at least 30%, at least 40%, at least 50%, at least 60%, or at least 70% greater. Without wishing to be limited by any particular theory, such increased magnitude of the therapeutic effect may indicate increased bioavailability. Such increased magnitude enables achieving a desired therapeutic effect on administering smaller doses of cannabinoids, saving thereby on cost.

According to an embodiment, the primary terpene is present in specific amounts, and the duration of the therapeutic effect, as measured by methods known in the art, is at least 20% longer compared with that of a composition comprising one half the amount of the primary terpene, at least 30%, at least 40%, at least 50%, at least 60%, at least 70%, at least 80%, at least 90% or at least 100% longer. According to an embodiment, the primary terpene and secondary terpenes are present in specific amounts, and the duration of the therapeutic effect is at least 20% longer than that of a composition comprising the same secondary terpene amounts and one half the amount of the primary terpene, at least 30%, at least 40%, at least 50%, at least 60%. at least 70%, at least 80%, at least 90% or at least 100% longer.

According to an embodiment, the primary terpene is present in specific amounts, and the frequency of the conditions and/or symptoms, as measured by methods known in the art, is at least 20% smaller compared with that of a composition comprising one half the amount of the primary terpene, at least 30%, at least 40%, at least 50% at least 60%. at least 70%, at least 80%, or at least 90% smaller. According to an embodiment, the primary terpene and secondary terpenes are present in specific amounts, and the frequency of the conditions and/or symptoms, as measured by methods known in the art, is at least 20% smaller than that of a composition comprising the same secondary terpene amounts and one half the amount of the primary terpene, at least 30%, at least 40%, at least 50%, at least 60%. at least 70%, at least 80%, at least 90% or at least 100% longer.

According to an embodiment, the primary terpene is present in specific amounts, and the severity of the conditions and/or symptoms, as measured by methods known in the art, is at least 20% smaller compared with that of a composition comprising one half the amount of the primary terpene, at least 30%, at least 40%, at least 50% at least 60%, at least 70%, at least 80%, or at least 90% smaller. According to an embodiment, the primary terpene and secondary terpenes are present in specific amounts, and the severity of the conditions and/or symptoms, as measured by methods known in the art, is at least 20% smaller than that of a composition comprising the same secondary terpene amounts and one half the amount of the primary terpene, at least 30%, at least 40%, at least 50%, at least 60%, at least 70%, at least 80%, at least 90% or at least 100% smaller.

According to an embodiment, the primary terpene is present in specific amounts, and the consumption of other drugs is reduced by at least 20% compared with that of a composition comprising one half the amount of said primary terpene, reduced by at least 30%, by at least 40%, by at least 50%, by at least 60%, by at least 70%, by at least 80%, or by at least 90%. According to an embodiment, the primary terpene and secondary terpenes are present in specific amounts, and the consumption of other drugs, as measured by methods known in the art, is reduced by at least 20% compared with a composition comprising the same secondary terpene amounts and one half the amount of the primary terpene, reduced by at least 30%, by at least 40%, by at least 50%, by at least 60%, by at least 70%, by at least 80%, or by at least 90%.

According to an embodiment said therapeutic effect treats osteoporosis and said the primary terpene is selected from the group consisting of caryophyllene, caryophyllene oxide, carene, eucalyptol, sabinene, pinene, limonene, borneol, thymol, camphor and combinations thereof. According to an embodiment, the composition comprises at least 2 of these terpenes, at least 3, at least 4, or at least 5 of the terpenes. According to an embodiment, the primary terpene is selected from the group consisting of caryophyllene, carene, borneol and combinations thereof. According to various embodiments said primary terpene comprises caryophyllene oxide, eucalyptol, sabinene, pinene, limonene, thymol and/or camphor. According to an embodiment, said therapeutic effect treats osteoporosis and the composition further comprises a compound selected from the group consisting of biochanin A, formonenetin, genistein, daidzein, glycitein, prenylnaringenin, diosgenin, acetin, cimicifugoside and combinations thereof.

According to an embodiment said therapeutic effect treats sleep disorder and/or insomnia and said the primary terpene is selected from the group consisting of linalool, caryophyllene, myrcene, terpineol, terpinene, citronellol, eucalyptol, nerolidol, sabinene, pinene, limonene, borneol, fenchol, bisabolol humulene, phytol and combinations thereof. According to an embodiment, the composition comprises at least 2 of these terpenes, at least 3, at least 4, or at least 5 of the terpenes. According to an embodiment, the primary terpene is selected from the group consisting of linalool, caryophyllene, myrcene and combinations thereof. According to an embodiment, the primary terpene is selected from the group consisting of linalool, nerolidol, myrcene and combinations thereof. According to various embodiments said primary terpene comprises terpineol, terpinene, citronellol, eucalyptol, nerolidol, sabinene, pinene, limonene, borneol, fenchol, bisabolol, phytol and/or humulene. According to an embodiment, said therapeutic effect treats sleep disorder and/or insomnia and the composition further comprises a compound selected from the group consisting of linalyl acetate, chamazulene, biochanin A, formonenetin, genistein, daidzein, glycitein, prenylnaringenin, diosgenin, acetin, cimicifugoside and combinations thereof. According to an embodiment, the composition includes caryophyllene, and caryophyllene forms less than 5% by weight of the total terpene content.

According to an embodiment said therapeutic effect treats anxiety, irritability, nervousness, restless, stress, depression, mood swing and/or affect disorder and said the primary terpene is selected from the group consisting of limonene, linalool, terpineol, citronellol, eucalyptol, caryophyllene, geraniol, menthol, iso-menthone, germacrene, pinene, citronellol, cardinol, neral, geranial, myrcene, terpinene, menth-2-en-1-ol, humulene, bisabolol, borneol, fenchol, nerolidol and combinations thereof. According to an embodiment, the composition comprises at least 2 of these terpenes, at least 3, at least 4, or at least 5 of the terpenes. According to an embodiment, the primary terpene is selected from the group consisting of limonene, linalool, eucalyptol and combinations thereof. According to an embodiment, the primary terpene is selected from the group consisting of limonene, linalool, caryophyllene, bisabolol and combinations thereof. According to an embodiment, the primary terpene is selected from the group consisting of limonene, linalool, pinene, humulene and combinations thereof. According to various embodiments said primary terpene comprises terpineol, citronellol, caryophyllene, geraniol, menthol, iso-menthone, germacrene, pinene, citronellol, cardinol, neral, geranial, myrcene, terpinene, menth-2-en-1-ol, humulene, bisabolol, borneol, nerolidol or fenchol. According to an embodiment, said therapeutic effect treats anxiety, irritability, nervousness, restless, stress, depression, mood swing and/or affect disorder and the composition further comprises a compound selected from the group consisting of linalyl acetate, geranyl acetate, menthol, piperine, eugenol, citronellyl formate, jasmone, benzy acetate, benzyl alcohol, biochanin A, formonenetin, genistein, daidzein, glycitein, prenylnaringenin, diosgenin, acetin, cimicifugoside and combinations thereof. According to an embodiment, the composition includes pinene, terpineol, bisabolol, caryophyllene, myrcene and/or limonene, and pinene, terpineol, bisabolol, caryophyllene, myrcene and/or limonene forms less than 5% by weight of the total terpene content.

According to an embodiment said therapeutic effect treats difficulty in concentrating, brain fog and/or memory loss and said the primary terpene is selected from the group consisting of eucalyptol, pinene, carene, limonene, pulegone, terpineol, cymene, linalool, caryophyllene and combinations thereof. According to an embodiment, the composition comprises at least 2 of these terpenes, at least 3, at least 4, or at least 5 of the terpenes. According to an embodiment, the primary terpene is selected from the group consisting of eucalyptol, pinene, limonene and combinations thereof. According to various embodiments said primary terpene comprises carene, pulegone, terpineol, linalool, caryophyllene and/or cymene According to an embodiment, said therapeutic effect treats difficulty in concentrating, brain fog and/or memory loss and the composition further comprises a compound selected from the group consisting of biochanin A, formonenetin, genistein, daidzein, glycitein, prenylnaringenin, diosgenin, acetin, cimicifugoside and combinations thereof.

According to an embodiment said therapeutic effect treats general discomfort and said the primary terpene is selected from the group consisting of limonene, linalool humulene, terpinene, borneol, caryophyllene and combinations thereof. According to an embodiment, the composition comprises at least 2 of these terpenes, at least 3, at least 4, or at least 5 of the terpenes. According to an embodiment, the primary terpene is selected from the group consisting of linalool, humulene, terpinene and combinations thereof. According to various embodiments said primary terpene comprises limonene, borneol and/or caryophyllene. According to an embodiment, the composition includes myrcene, pinene and/or bisabolol, and myrcene, pinene and/or bisabolol forms less than 5% by weight of the total terpene content.

According to an embodiment said therapeutic effect treats muscle tension and/or myalgia and said the primary terpene is selected from the group consisting of myrcene, pinene, nerolidol, linalool, humulene, caryophyllene, limonene, bisabolol and combinations thereof. According to an embodiment, the composition comprises at least 2 of these terpenes, at least 3, at least 4, or at least 5 of the terpenes. According to an embodiment, the primary terpene is selected from the group consisting of myrcene, pinene, nerolidol and combinations thereof. According to an embodiment, the primary terpene is selected from the group consisting of linalool, humulene, nerolidol and combinations thereof. According to an embodiment, the primary terpene is selected from the group consisting of myrcene, pinene, nerolidol, linalool and combinations thereof. According to various embodiments said primary terpene comprises linalool, humulene, caryophyllene, limonene and/or bisabolol.

According to an embodiment said therapeutic effect treats fatigue and/or intermittent dizziness and said the primary terpene is selected from the group consisting of pinene, limonene, terpinene, eucalyptol, myrcene, terpineol, linalool, caryophyllene and combinations thereof. According to an embodiment, the composition comprises at least 2 of these terpenes, at least 3, at least 4, or at least 5 of the terpenes. According to an embodiment, the primary terpene is selected from the group consisting of pinene, limonene, eucalyptol and combinations thereof. According to various embodiments said primary terpene comprises terpinene, myrcene and/or caryophyllene.

According to an embodiment said therapeutic effect treats menopausal skin, itchy skin, age-related wrinkles and/or stretch marks and said the primary terpene is selected from the group consisting of linalool limonene, nerolidol, pinene terpineol, myrcene, geraniol, nerol, citronellol, farnesol, carotol and combinations thereof. According to an embodiment, the composition comprises at least 2 of these terpenes, at least 3, at least 4, or at least 5 of the terpenes. According to an embodiment, the primary terpene is selected from the group consisting of linalool, terpineol, geraniol and combinations thereof. According to various embodiments said primary terpene comprises limonene, nerolidol, pinene myrcene, nerol, citronellol, farnesol and/or carotol. According to an embodiment, said therapeutic effect treats menopausal skin, itchy skin, age-related wrinkles and/or stretch marks and the composition further comprises a compound selected from the group consisting of linalyl acetate, citronellyl formate, eugenol, benzy acetate, benzyl alcohol, biochanin A, formonenetin, genistein, daidzein, glycitein, prenylnaringenin, diosgenin, acetin, cimicifugoside and combinations thereof.

According to an embodiment said therapeutic effect treats headache and/or migraine and said the primary terpene is selected from the group consisting of myrcene, linalool, caryophyllene, eucalyptol, limonene, nerolidol, fenchol, humulene, guaiol, borneol, pinene, bisabolol, terpinene, terpineol and combinations thereof. According to an embodiment, the composition comprises at least 2 of these terpenes, at least 3, at least 4, or at least 5 of the terpenes. According to an embodiment, the primary terpene is selected from the group consisting of myrcene, linalool, caryophyllene and combinations thereof. According to various embodiments said primary terpene comprises eucalyptol, limonene, nerolidol, fenchol, humulene, guaiol, linalool, limonene, borneol, pinene and/or bisabolol. According to an embodiment, the composition includes bisabolol, terpinene, humulene and/or linalool wherein bisabolol, terpinene, humulene and/or linalool forms less than 5% by weight of the total terpene content.

According to an embodiment said therapeutic effect treats weight gain and said the primary terpene is selected from the group consisting of eucalyptol, caryophyllene, limonene, humulene, phytol and combinations thereof. According to an embodiment, the composition comprises at least 2 of these terpenes, at least 3, at least 4, or at least 5 of the terpenes. According to an embodiment, the primary terpene is selected from the group consisting of eucalyptol, limonene, humulene and combinations thereof. According to various embodiments said primary terpene comprises caryophyllene and/or phytol.

According to an embodiment said therapeutic effect treats digestive problems, intestinal disorders, abdominal pain (colic), nausea, constipation and/or diarrhea and said the primary terpene is selected from the group consisting of limonene, cymene, terpinene, caryophyllene, citronellol, sabinene, myrcene, linalool, germacrene, geraniol, pinene, terpineol and combinations thereof. According to an embodiment, the composition comprises at least 2 of these terpenes, at least 3, at least 4, or at least 5 of the terpenes. According to an embodiment, the primary terpene is selected from the group consisting of limonene, sabinene, myrcene and combinations thereof. According to various embodiments said primary terpene comprises cymene, terpinene, caryophyllene, citronellol, linalool, germacrene, geraniol, terpineol and/or pinene. According to an embodiment, said therapeutic effect treats digestive problems, intestinal disorders, abdominal pain (colic), nausea, constipation and/or diarrhea and the composition further comprises piperine. According to an embodiment, the composition includes pinene, bisabolol, limonene and/or terpineol, wherein pinene, bisabolol, limonene and/or terpineol forms less than 5% by weight of the total terpene content

According to an embodiment said therapeutic effect treats pain and/or Mastalgia and said the primary terpene is selected from the group consisting of pinene, limonene, caryophyllene, myrcene, linalool, geraniol, eucalyptol, terpineol, sabinene and combinations thereof. According to an embodiment, the composition comprises at least 2 of these terpenes, at least 3, at least 4, or at least 5 of the terpenes. According to an embodiment, the primary terpene is selected from the group consisting of pinene, linalool, eucalyptol and combinations thereof. According to various embodiments said primary terpene comprises limonene, caryophyllene, myrcene, geraniol, terpineol and/or sabinene. According to an embodiment, said therapeutic effect treats pain and/or Mastalgia and the composition further comprises a compound selected from the group consisting of biochanin A, formonenetin, genistein, daidzein, glycitein, prenylnaringenin, diosgenin, acetin, cimicifugoside and combinations thereof.

According to an embodiment said therapeutic effect treats dysmenorrhea, pelvic pain, spasms, cramps, labor contraction and/or endometriosis and said the primary terpene is selected from the group consisting of myrcene, linalool, caryophyllene, eucalyptol, nerolidol, limonene, nerolidol, sabinene, humulene, terpineol, citronellol, geraniol, pinene, limonene, camphene, terpinene, borneol, camphor, menthone, iso-menthone, carvacrol, thymol, carene, nerol, farnesol, menth-2-en-1-ol and combinations thereof. According to an embodiment, the composition comprises at least 2 of these terpenes, at least 3, at least 4, or at least 5 of the terpenes. According to an embodiment, the primary terpene is selected from the group consisting of linalool, eucalyptol, nerolidol and combinations thereof. According to an embodiment, the primary terpene is selected from the group consisting of linalool, myrcene, nerolidol and combinations thereof. According to various embodiments said primary terpene comprises myrcene, caryophyllene, limonene, nerolidol, sabinene, humulene, terpineol, citronellol, geraniol, pinene, limonene, camphene, terpinene, borneol, camphor, menthone, iso-menthone, carvacrol, thymol, carene, nerol, farnesol and/or menth-2-en-ol. According to an embodiment, said therapeutic effect treats dysmenorrhea, pelvic pain, spasms, cramps, labor contraction and/or endometriosis and the composition further comprises a compound selected from the group consisting of linalyl acetate, geranyl acetate, eugenol, methylis-oeugenol, menthol, eugenyl acetate, piperine, chamazulene, citronllyl formate, benzyl acetate, benzyl alcohol, biochanin A, formonenetin, genistein, daidzein, glycitein, prenylnaringenin, diosgenin, acetin, cimicifugoside and combinations thereof.

According to an embodiment said therapeutic effect treats arthralgia and/or arthritis and said the primary terpene is selected from the group consisting of myrcene, linalool, guaiol, caryophyllene, eucalyptol, pinene, nerolidol, sabinene, limonene, terpinene, menth-2-en-1-ol, geraniol, guaiol, humulene, bisabolol, borneol, fenchol, terpineol and combinations thereof. According to an embodiment, the composition comprises at least 2 of these terpenes, at least 3, at least 4, or at least 5 of the terpenes. According to an embodiment, the primary terpene is selected from the group consisting of myrcene, pinene, sabinene and combinations thereof. According to an embodiment, the primary terpene is selected from the group consisting of terpineol, linalool, sabinene and combinations thereof. According to various embodiments said primary terpene comprises linalool, guaiol, caryophyllene, eucalyptol, nerolidol, limonene, terpinene, menth-2-en-1-ol, geraniol, guaiol, humulene, bisabolol, borneol, fenchol and/or terpineol. According to an embodiment, said therapeutic effect treats arthralgia and/or arthritis and the composition further comprises a compound selected from the group consisting of linalyl acetate, geranyl acetate, eugenol, methylis-oeugenol, menthol, eugenyl acetate, piperine and combinations thereof. According to an embodiment, the composition optionally includes humulene, pinene, bisabolol, borneol, fenchol, guaiol, linalool, ocimene, terpineol, terpinene and/or caryophyllene, wherein humulene, pinene, bisabolol, borneol, fenchol, guaiol, linalool, ocimene, terpineol, terpinene and/or caryophyllene forms less than 5% by weight of the total terpene content.

According to an embodiment said therapeutic effect treats hot flashes, facial flushing, night sweat, irritability, palpitation and/or tachycardia and said the primary terpene is selected from the group consisting of caryophyllene, myrcene, humulene, linalool, terpineol, geraniol, pinene, carene, citronellol, limonene, eucalyptol, sabinene, myrtenol, terpinene, champhene, borneol and combinations thereof. According to an embodiment, the composition comprises at least 2 of these terpenes, at least 3, at least 4, or at least 5 of the terpenes. According to an embodiment, the primary terpene is selected from the group consisting of linalool, pinene, eucalyptol and combinations thereof. According to an embodiment, the primary terpene is selected from the group consisting of linalool, geraniol, limonene, eucalyptol and combinations thereof. According to various embodiments said primary terpene comprises caryophyllene, myrcene, humulene, terpineol, geraniol, pinene, carene, citronellol, limonene, sabinene, myrtenol, champhene, borneol and/or terpinene. According to an embodiment, said therapeutic effect treats hot flashes, facial flushing, night sweat, irritability, palpitation and/or tachycardia and the composition further comprises a compound selected from the group consisting of linalyl acetate, geranyl acetate, citronellyl formate, biochanin A, formonenetin, genistein, daidzein, glycitein, prenylnaringenin, diosgenin, acetin, cimicifugoside and combinations thereof.

According to an embodiment said therapeutic effect treats inflammation, oxidative stress and/or paresthesia and said the primary terpene is selected from the group consisting of caryophyllene, pinene, myrcene, limonene, humulene, citronellol, eucalyptol, linalool, beta amyrin, cycloartenol, terpinene, terpineol, geraniol, carvacrol, thymol, cardinol, terpinene, menth-2-en-1-ol, carotol, humulene, nerolidol and combinations thereof. According to an embodiment, the composition comprises at least 2 of these terpenes, at least 3, at least 4, or at least 5 of the terpenes. According to an embodiment, the primary terpene is selected from the group consisting of caryophyllene, pinene, eucalyptol and combinations thereof. According to various embodiments said primary terpene comprises myrcene, limonene, humulene, citronellol, linalool, beta amyrin, cycloartenol, terpinene, terpineol, geraniol, carvacrol, thymol, cardinol, terpinene, menth-2-en-1-ol, carotol, humulene and/or nerolidol. According to an embodiment, said therapeutic effect treats inflammation, oxidative stress and/or paresthesia and the composition further comprises a compound selected from the group consisting of linalyl acetate, geranyl acetate, eugenol, eugenyl acetate, piperine and combinations thereof.

According to an embodiment said therapeutic effect treats cardiovascular disease, palpitation and tachycardia and said the primary terpene is selected from the group consisting of linalool, camphene, limonene, pinene, borneol and combinations thereof. According to an embodiment, the composition comprises at least 2 of these terpenes, at least 3, at least 4, or all 5 of the terpenes. According to an embodiment, the primary terpene is selected from the group consisting of linalool, limonene, pinene and combinations thereof. According to various embodiments said primary terpene comprises camphene and/or borneol.

According to an embodiment said therapeutic effect treats urine incontinence and/or lower urinary tract disorders and said the primary terpene is selected from the group consisting of linalool, terpineol, pinene, carene, geraniol, thujone, camphor, viridiflorol, caryophyllene, germacrene, cirtonellol, eucalyptol, myrtenol, sanatalol, sabinene, limonene and combinations thereof. According to an embodiment, the composition comprises at least 2 of these terpenes, at least 3, at least 4, or at least 5 of the terpenes. According to an embodiment, the primary terpene is selected from the group consisting of linalool, geraniol, pinene and combinations thereof. According to various embodiments said primary terpene comprises terpineol, carene, thujone, camphor, viridiflorol, caryophyllene, germacrene, cirtonellol, eucalyptol, myrtenol, sanatalol, sabinene and/or limonene According to an embodiment, said therapeutic effect treats urine incontinence and/or lower urinary tract disorders and the composition further comprises a compound selected from the group consisting of linalyl acetate, geranyl acetate, citronellyl formate and combinations thereof.

According to an embodiment said therapeutic effect treats vaginal dryness and said the primary terpene is selected from the group consisting of myrcene, humulene, caryophyllene, linalool, terpineol, geraniol, pinene, germacrene, cirtonellol, limonene, myrcene, terpinene, menth-2-en-1-ol, eucalyptol, myrtenol and combinations thereof. According to an embodiment, the composition comprises at least 2 of these terpenes, at least 3, at least 4, or at least 5 of the terpenes. According to an embodiment, the primary terpene is selected from the group consisting of linalool, geraniol, pinene and combinations thereof. According to various embodiments said primary terpene myrcene, humulene, caryophyllene, terpineol, germacrene, cirtonellol, limonene, myrcene, terpinene, menth-2-en-1-ol, eucalyptol and/or myrtenol. According to an embodiment, said therapeutic effect treats vaginal dryness and the composition further comprises a compound selected from the group consisting of linalyl acetate, geranyl acetate, citronellyl formate, biochanin A, formonenetin, genistein, daidzein, glycitein, prenylnaringenin, diosgenin, acetin, cimicifugoside and combinations thereof.

According to an embodiment said therapeutic effect treats female urogenital and/or reproductive system infection and said the primary terpene is selected from the group consisting of pinene, limonene, cymene, caryophyllene oxide, ocimene, nerolidol, eucalyptol, terpinene, terpineol, camphor, linalool, sabinene, thujone, camphor, viridiflorol, menthol, iso-menthone, caryophyllene, germacrene, geraniol, nerol, neral, geranial, citronellol, carvacol, thymol, carene, myrtenol, carene, myrcene, menth-2-en-1-ol and combinations thereof. According to an embodiment, the composition comprises at least 2 of these terpenes, at least 3, at least 4, or at least 5 of the terpenes. According to an embodiment, the primary terpene is selected from the group consisting of caryophyllene oxide, pinene, limonene, geraniol and combinations thereof. According to an embodiment, the primary terpene is selected from the group consisting of caryophyllene oxide, pinene, limonene and combinations thereof. According to various embodiments said primary terpene comprises cymene, ocimene, nerolidol, eucalyptol, terpinene, terpineol, camphor, linalool, sabinene, thujone, camphor, viridiflorol, menthol, iso-menthone, caryophyllene, germacrene, geraniol, nerol, neral, geranial, citronellol, carvacol, thymol, carene, myrtenol, carene, myrcene and/or menth-2-en-1-ol. According to an embodiment, said therapeutic effect treats female urogenital and/or reproductive system infection and the composition further comprises a compound selected from the group consisting of linalyl acetate, geranyl acetate, eugenol, eugenyl acetate, piperine, menthol, citronellyl formate and combinations thereof. According to an embodiment the therapeutic effect treats human papillomavirus (HPV).

According to an embodiment said therapeutic effect treats fibroid, myomas and/or cysts in the female genital system and said the primary terpene is selected from the group consisting of linalool, terpineol, limonene, pinene, caryophyllene, caryophyllene oxide, terpinene, citronellol and combinations thereof. According to an embodiment, the composition comprises at least 2 of these terpenes, at least 3, at least 4, or at least 5 of the terpenes. According to an embodiment, the primary terpene is selected from the group consisting of limonene, pinene, terpinene and combinations thereof. According to various embodiments said primary terpene comprises linalool, terpineol, caryophyllene, caryophyllene oxide and/or citronellol.

According to an embodiment said therapeutic effect treats cystitis, urethritis and/or pyelonephritis and said the primary terpene is selected from the group consisting of thujone, pinene, limonene, cymene, caryophyllene, caryophyllene oxide, eucalyptol, terpineol, sabinene, linalool, cardinol and combinations thereof. According to an embodiment, the composition comprises at least 2 of these terpenes, at least 3, at least 4, or at least 5 of the terpenes. According to an embodiment, the primary terpene is selected from the group consisting of limonene, eucalyptol, linalool and combinations thereof. According to an embodiment, the primary terpene is selected from the group consisting of limonene, pinene, linalool and combinations thereof. According to various embodiments said primary terpene comprises thujone, pinene, cymene, caryophyllene, caryophyllene oxide, terpineol, sabinene and/or cardinol.

According to an embodiment said therapeutic effect treats libido and/or sexual dysfunction and said the primary terpene is selected from the group consisting of limonene, linalool, terpineol, geraniol, eucalyptol, pinene, citronellol and combinations thereof. According to an embodiment, the composition comprises at least 2 of these terpenes, at least 3, at least 4, or at least 5 of the terpenes. According to an embodiment, the primary terpene is selected from the group consisting of limonene, linalool, ecualyptol and combinations thereof. According to various embodiments said primary terpene comprises terpineol, geraniol and/or pinene, cintronellol According to an embodiment, said therapeutic effect treats libido and/or sexual disfunction and the composition further comprises a compound selected from the group consisting of biochanin A, formonenetin, genistein, daidzein, glycitein, prenylnaringenin, diosgenin, acetin, cimicifugoside and combinations thereof.

According to an embodiment said therapeutic effect treats hair loss and/or thinning and said the primary terpene is selected from the group consisting of linalool, terpineol, eucalyptol, borneol, camphor, pinene, geraniol and combinations thereof. According to an embodiment, the composition comprises at least 2 of these terpenes, at least 3, at least 4, or at least 5 of the terpenes. According to an embodiment, the primary terpene is selected from the group consisting of geraniol, eucalyptol, terpineol and combinations thereof. According to various embodiments said primary terpene comprises linalool, borneol, camphor and/or pinene.

According to an embodiment said therapeutic effect treats brittle nails and said the primary terpene is selected from the group consisting of linalool, eucalyptol, geraniol, pinene, limonene and combinations thereof. According to an embodiment, the composition comprises at least 2 of these terpenes, at least 3, at least 4, or all 5 of the terpenes. According to an embodiment, the primary terpene is selected from the group consisting of linalool, eucalyptol, limonene and combinations thereof. According to various embodiments said primary terpene comprises geraniol and/or pinene.

According to an embodiment said therapeutic effect treats irregular body odor and said the primary terpene is selected from the group consisting of linalool, terpineol, geraniol, eucalyptol and combinations thereof. According to an embodiment, the composition comprises at least 2 of these terpenes, at least 3, or all 4 of the terpenes. According to an embodiment, the primary terpene is selected from the group consisting of linalool, terpineol, geraniol and combinations thereof.

According to an embodiment said therapeutic effect treats Polycystic Ovary Syndrome (POCS), amenorrhea, oligomenorrhea, hypermenorrhea, metrorrhagia and said the primary terpene is selected from the group consisting of humulene, linalool, terpineol, caryophyllene, caryophyllene oxide, terpinene, citronellol, geraniol, thujone, camphor, viridiflorol, pinene, camphene, sabinene, myrcene, eucalyptol, carene, carotol, limonene, sabinene, cardinal, pinene and combinations thereof. According to an embodiment, the composition comprises at least 2 of these terpenes, at least 3, at least 4, or at least 5 of the terpenes. According to an embodiment, the primary terpene is selected from the group consisting of linalool, eucalyptol, geraniol and combinations thereof. According to an embodiment, the primary terpene is selected from the group consisting of linalool, eucalyptol, caryophyllene, pinene and combinations thereof. According to various embodiments said primary terpene comprises humulene, terpineol, caryophyllene, caryophyllene oxide, terpinene, citronellol, thujone, camphor, viridiflorol, pinene, camphene, sabinene, myrcene, carene, carotol, limonene, sabinene, cardinal and/or pinene. According to an embodiment, said therapeutic effect treats Polycystic Ovary Syndrome (POCS), amenorrhea, oligomenorrhea, hypermenorrhea, metrorrhagia and the composition further comprises a compound selected from the group consisting of linalyl acetate, geranyl acetate, eugenol, chamazulene, citronellyl formate and combinations thereof.

According to an embodiment said therapeutic effect treats premenstrual syndrome (PMS), premenstrual tension, and/or premenstrual dysphoric disorder (PMDD) and said the primary terpene is selected from the group consisting of limonene, linalool, eucalyptol, nerolidol, sabinene, pinene, caryophyllene, terpineol, geraniol, thujone, camphor, viridiflorol, champhene, myrcene, caryophyllene, terpinene, carene, cirtonellol, carotol, neral, geranial and combinations thereof. According to an embodiment, the composition comprises at least 2 of these terpenes, at least 3, at least 4, or at least 5 of the terpenes. According to an embodiment, the primary terpene is selected from the group consisting of linalool, eucalyptol, geraniol and combinations thereof. According to an embodiment, the primary terpene is selected from the group consisting of linalool, caryophyllene, limonene and combinations thereof. According to an embodiment, the primary terpene is selected from the group consisting of linalool, caryophyllene, limonene, eucalyptol, geraniol and combinations thereof. According to various embodiments said primary terpene comprises limonene, nerolidol, sabinene, pinene, caryophyllene, terpineol, thujone, camphor, viridiflorol, champhene, myrcene, caryophyllene, terpinene, carene, cirtonellol, carotol, neral and/or geranial. According to an embodiment, said therapeutic effect treats premenstrual syndrome (PMS), premenstrual tension, and/or premenstrual dysphoric disorder (PMDD) and the composition further comprises a compound selected from the group consisting of linalyl acetate, geranyl acetate, citronellyl formate, menthol, eugenol piperine, jasmone and combinations thereof.

According to an embodiment said therapeutic effect treats hormonal disfunction and/or imbalance and/or fertility and said the primary terpene is selected from the group consisting of linalool, terpineol, geraniol, thujone, camphor, viridiflorol, borneol, champhene, pinene, eucalyptol, caryophyllene, germacrene, pinene, carene, cirtonellol, nerol, farnesol, limonene, myrcene, terpinene, menth-2-en-1-ol, carotol, sabinene, neral, geranial and combinations thereof. According to an embodiment, the composition comprises at least 2 of these terpenes, at least 3, at least 4, or at least 5 of the terpenes. According to an embodiment, the primary terpene is selected from the group consisting of linalool, eucalyptol, geraniol and combinations thereof. According to an embodiment, the primary terpene is selected from the group consisting of linalool, eucalyptol, geraniol, terpineol and combinations thereof. According to an embodiment, the primary terpene is selected from the group consisting of linalool, eucalyptol, geraniol, pinene, caryophyllene and combinations thereof. According to various embodiments said primary terpene comprises terpineol, geraniol, thujone, camphor, viridiflorol, borneol, champhene, pinene, caryophyllene, germacrene, pinene, carene, cirtonellol, nerol, farnesol, limonene, myrcene, terpinene, menth-2-en-1-ol, carotol, sabinene, neral and/or geranial. According to an embodiment, said therapeutic effect treats hormonal disfunction and/or imbalance and/or fertility and the composition further comprises a compound selected from the group consisting of linalyl acetate, geranyl acetate, chamazulene, citronellyl formate, biochanin A, formonenetin, genistein, daidzein, glycitein, prenylnaringenin, sclareol, diosgenin, acetin, cimicifugoside and combinations thereof.

According to an embodiment said therapeutic effect treats pre-eclampsia toxemia and said the primary terpene is selected from the group consisting of limonene, neral, geranial, caryophyllene, linalool, myrcene, nerolidol, fenchol, humulene, guaiol, borneol, cymene, terpinene and combinations thereof. According to an embodiment, the composition comprises at least 2 of these terpenes, at least 3, at least 4, or at least 5 of the terpenes. According to an embodiment, the primary terpene is selected from the group consisting of linalool, myrcene, limonene and combinations thereof. According to an embodiment, the primary terpene is selected from the group consisting of linalool, myrcene, limonene, caryophyllene and combinations thereof. According to various embodiments said primary terpene comprises neral, geranial, caryophyllene, nerolidol, fenchol, humulene, guaiol, borneol, cymene and/or terpinene.

According to an embodiment said therapeutic effect treats conditions and/or symptoms associated with female hormonal disfunction and/or imbalance and said the primary terpene is selected from the group consisting of linalool, terpineol, geraniol, thujone, camphor, viridiflorol, borneol, champhene, pinene, eucalyptol, caryophyllene, germacrene, pinene, carene, cirtonellol, nerol, farnesol, limonene, myrcene, terpinene, menth-2-en-1-ol, carotol, sabinene, neral, geranial, nerolidol, terpineol, carene and combinations thereof. According to an embodiment, the composition comprises at least 2 of these terpenes, at least 3, at least 4, or at least 5 of the terpenes. According to an embodiment, the primary terpene is selected from the group consisting of geraniol, linalool, eucalyptol and combinations thereof. According to an embodiment, the primary terpene is selected from the group consisting of terpineol, linalool, caryophyllene, pinene and combinations thereof. According to an embodiment, the primary terpene is selected from the group consisting of geraniol, linalool, eucalyptol, caryophyllene, terpineol, pinene, limonene and combinations thereof. According to various embodiments said primary terpene comprises terpineol, geraniol, thujone, camphor, viridiflorol, borneol, champhene, pinene, eucalyptol, caryophyllene, germacrene, pinene, carene, cirtonellol, nerol, farnesol, limonene, myrcene, terpinene, menth-2-en-1-ol, carotol, sabinene, neral, geranial, nerolidol, terpineol and/or carene. According to an embodiment, said therapeutic effect treats conditions and/or symptoms associated with female hormonal disfunction and/or imbalance and the composition further comprises a compound selected from the group consisting of biochanin A, formonenetin, genistein, daidzein, glycitein, prenylnaringenin, diosgenin, acetin, cimicifugoside and combinations thereof.

According to an embodiment said therapeutic effect treats conditions and/or symptoms associated with perimenopause and/or menopause and said the primary terpene is selected from the group consisting of caryophyllene, caryophyllene oxide, carene, linalool, myrcene, terpinene, terpineol, citronellol, eucalyptol, nerolidol, limonene, geraniol, menthol, iso-menthone, germacrene, pinene, cardinol, neral, geranial, menth-2-en-1-ol, pulegone, cymene, fenchol, humulene, guaiol, borneol, carene, sabinene, myrtenol, camphene, nerol, farnesol, carotol and combinations thereof. According to an embodiment, the composition comprises at least 2 of these terpenes, at least 3, at least 4, or at least 5 of the terpenes. According to an embodiment, the primary terpene is selected from the group consisting of caryophyllene, linalool, carene and combinations thereof. According to an embodiment, the primary terpene is selected from the group consisting of caryophyllene, linalool, myrcene and combinations thereof. According to an embodiment, the primary terpene is selected from the group consisting of nerolidol, limonene, linalool and combinations thereof. According to an embodiment, the primary terpene is selected from the group consisting of caryophyllene, linalool, carene, nerolidol, limonene and combinations thereof. According to various embodiments said primary terpene comprises caryophyllene, caryophyllene oxide, carene, myrcene, terpinene, terpineol, citronellol, eucalyptol, nerolidol, limonene, geraniol, menthol, iso-menthone, germacrene, pinene, cardinol, neral, geranial, menth-2-en-1-ol, pulegone, cymene, fenchol, humulene, guaiol, borneol, carene, sabinene, myrtenol, camphene, nerol, farnesol and/or carotol. According to an embodiment, said therapeutic effect treats conditions and/or symptoms associated with perimenopause and/or menopause and the composition further comprises a compound selected from the group consisting of biochanin A, formonenetin, genistein, daidzein, glycitein, prenylnaringenin, diosgenin, acetin, cimicifugoside and combinations thereof.

According to an embodiment said therapeutic effect treats pain and/or cramps and said the primary terpene is selected from the group consisting of myrcene, linalool, caryophyllene, eucalyptol, nerolidol, sabinene, humulene, terpineol, citronellol, geraniol, pinene, limonene, camphene, terpinene, borneol, camphor, menthone, iso-menthone, carvacrol, thymol, carene, nerol, farnesol, menth-2-en-1-ol, guaiol and combinations thereof. According to an embodiment, the composition comprises at least 2 of these terpenes, at least 3, at least 4, or at least 5 of the terpenes. According to an embodiment, the primary terpene is selected from the group consisting of limonene, linalool, eucalyptol and combinations thereof. According to an embodiment, the primary terpene is selected from the group consisting of limonene, linalool, eucalyptol, myrcene and combinations thereof. According to various embodiments said primary terpene comprises myrcene, caryophyllene, nerolidol, sabinene, humulene, terpineol, citronellol, geraniol, pinene, camphene, terpinene, borneol, camphor, menthone, iso-menthone, carvacrol, thymol, carene, nerol, farnesol, menth-2-en-1-ol and/or guaiol.

According to an embodiment said therapeutic effect treats female urogenital and/or reproductive system infections and/or disorders and said the primary terpene is selected from the group consisting of myrcene, humulene, caryophyllene, linalool, terpineol, geraniol, pinene, germacrene, cirtonellol, limonene, myrcene, terpinene, menth-2-en-1-ol, borneol, eucalyptol, myrtenol, cymene, caryophyllene oxide, ocimene, nerolidol, camphor, sabinene, thujone, camphor, viridiflorol, menthol, iso-menthone, caryophyllene, germacrene, geraniol, nerol, neral, geranial, citronellol, carvacol, thymol, carene, myrtenol, carene, thujone and combinations thereof. According to an embodiment, the composition comprises at least 2 of these terpenes, at least 3, at least 4, or at least 5 of the terpenes. According to an embodiment, the primary terpene is selected from the group consisting of linalool, pinene, caryophyllene oxide and combinations thereof. According to an embodiment, the primary terpene is selected from the group consisting of geraniol, linalool, pinene, caryophyllene oxide and combinations thereof. According to various embodiments said primary terpene comprises myrcene, humulene, caryophyllene, terpineol, geraniol, germacrene, cirtonellol, limonene, myrcene, terpinene, menth-2-en-1-ol, borneol, eucalyptol, myrtenol, cymene, ocimene, nerolidol, camphor, sabinene, thujone, camphor, viridiflorol, menthol, iso-menthone, caryophyllene, germacrene, geraniol, nerol, neral, geranial, citronellol, carvacol, thymol, carene, myrtenol, carene and/or thujone.

According to an embodiment, the method for treating a patient comprises administering to said patient a product selected from the group consisting of tablets, gel capsules, medical patches, topicals, creams, varnishes, sublingual oils, sprays, edibles, suppositories including tampons and/or rectal candles, cigarettes, vaporizer liquids, micro and nano-emulsion preparations containing micro and nano-particle, and combinations thereof, containing the administered composition. According to an embodiment, the product comprising tablets containing the composition. According to an embodiment, the product comprising gel capsules containing the composition. According to an embodiment, the product comprising medical patches containing the composition. According to an embodiment, the product comprising topicals containing the composition. According to an embodiment, the product comprising creams containing the composition. According to an embodiment, the product comprising varnishes containing the composition. According to an embodiment, the product comprising sublingual oils containing the composition. According to an embodiment, the product comprising edibles containing the composition. According to an embodiment, the product comprising tampons containing the composition. A According to an embodiment, the product comprising rectal candles containing the composition. According to an embodiment, the product comprising cigarettes containing the composition. According to an embodiment, the product comprising vaporizer liquids containing the composition.

A composition comprising five or less terpenes in a concentration of at least 10% of the total terpene content, forming a terpene composition with enhanced therapeutic effect for treating conditions and/or symptoms associated with perimenopause, menopause, female urogenital or reproductive system infections and/or disorders, menstrual cycle, hormonal deficiency and/or hormonal imbalance. A composition comprising five or less terpenes in a concentration of at least 10%, at least 12%, at least 15%, at least 20%, at least 25%, at least 30%, at least 35%, at least 40%, at least 45%, at least 50%, at least 55%, at least 60%, or at least 70% of the total terpene content. According to an embodiment, the conditions and/or symptoms are selected from the group consisting of hot flashes, facial flushing, night sweat, osteoporosis, fatigue, irritability, insomnia and/or sleep disorder, difficulty in concentration, depression, memory loss, brain fog, headache, migraine, anxiety, nervousness, restless, stress, mood swing, affect disorder, general discomfort, muscle tension, myalgia, intermittent dizziness, paresthesia, indigestion, digestive problems, intestinal disorders, abdominal pain, irritability, palpitations, tachycardia, nausea, constipation, diarrhea, arthralgia and/or arthritis, dysmenorrheal, pelvic pain, spasms, cramps, labor contraction, endometriosis Polycystic Ovary Syndrome (POCS), amenorrhea, oligomenorrhea, hypermenorrhea, metrorrhagia, premenstrual syndrome (PMS), premenstrual tension, premenstrual dysphoric disorder (PMDD), cold hands and feet, weight gain, mental awareness problems, sexual pain disorder, dyspareunia, breast pain and/or mastalgia, menopausal skin, inflammation, oxidative stress, cardiovascular disease, loss of pelvic muscle tone, urinary incontinence, lower urinary tract disorders, vaginal dryness, women urogenital and/or reproductive system infection, fibroid, myomas and/or cysts in the female genital system, human papillomavirus (HPV), Uterine Fibroid, cystitis, urethritis, pyelonephritis, women hormonal dysfunction and/or imbalance, irregular periods, pre-eclampsia toxemia, reduced libido, reduced sexual dysfunction, hair loss and/or thinning, age-related winkles, stretch marks, brittle nails, gam disorders, irregular body odor, conditions and/or symptoms induced by estrogen deficiency and combinations thereof.

According to an embodiment, the primary terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thujone, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

According to an embodiment, the composition additionally contains an additive selected from the group consisting of antioxidants, emulsifiers and texturizers vegetable oils, plant extracts, honey, sucrose, glucose and fructose, pharmaceutical excipients and combinations thereof.

According to an embodiment, the composition comprises less than 1% THC, less than 0.8%, less than 0.6%, less than 0.4%, less than 0.2%, or less than 0.1% THC.

According to an embodiment, the composition comprises at least one of isoflavones, coumestans, prenylflavonoids, lignans and saponins. According to an embodiment, the composition comprises isoflavones. According to an embodiment, the composition comprises coumestans. According to an embodiment, the composition comprises prenylflavonoids. According to an embodiment, the composition comprises lignans. According to an embodiment, the composition comprises saponins. According to an embodiment, the composition comprises at least one of biochanin A, formonenetin, genistein, daidzein, glycitein, prenylnaringenin, diosgenin, acetin and/or cimicifugoside. According to an embodiment, the composition comprises biochanin A. According to an embodiment, the composition comprises formonenetin. According to an embodiment, the composition comprises genistein. According to an embodiment, the composition comprises daidzein. According to an embodiment, the composition comprises glycitein. According to an embodiment, the composition comprises prenylnaringenin. According to an embodiment, the composition comprises diosgenin. According to an embodiment, the composition comprises acetin. According to an embodiment, the composition comprises cimicifugoside.

According to an embodiment said therapeutic effect treats osteoporosis and said compositon comprises caryophyllene, carene, borneol, thymol and/or camphor. According to an embodiment, the composition comprises at least 2 of these terpenes, at least 3, at least 4, or all 5 of the terpenes. According to an embodiment said therapeutic effect treats osteoporosis and said compositon comprises caryophyllene, carene, borneol, camphor and combinations thereof. According to an embodiment said therapeutic effect treats osteoporosis and said compositon comprises caryophyllene, carene, borneol and combinations thereof. According to various embodiments said composition comprises caryophyllene oxide, eucalyptol, sabinene, pinene and/or limonene.

According to an embodiment said therapeutic effect treats sleep disorder and/or insomnia and said compositon comprises linalool, caryophyllene, myrcene, terpinene and/or borneol. According to an embodiment, the composition comprises at least 2 of these terpenes, at least 3, at least 4, or all 5 of the terpenes. According to an embodiment, the composition comprises linalool, caryophyllene, myrcene, terpinene and combinations thereof. According to an embodiment, the composition comprises linalool, caryophyllene, myrcene and combinations thereof. According to an embodiment, the composition comprises linalool, nerolidol, myrcene and combinations thereof. According to various embodiments said composition comprises terpineol, terpinene, citronellol, eucalyptol, nerolidol, sabinene, pinene, limonene, borneol, fenchol, bisabolol and/or humulene. According to an embodiment, the composition includes caryophyllene, and caryophyllene forms less than 5% by weight of the total terpene content.

According to an embodiment said therapeutic effect treats anxiety, irritability, nervousness, restless, stress, depression, mood swing and/or affect disorder and said compositon comprises limonene, linalool, eucalyptol, caryophyllene and/or myrcene. According to an embodiment, the composition comprises at least 2 of these terpenes, at least 3, at least 4, or all 5 of the terpenes. According to an embodiment, the compositon comprises limonene, linalool, eucalyptol, myrcene and combinations thereof. According to an embodiment, the the compositon comprises limonene, linalool, caryophyllene, bisabolol and combinations thereof. According to an embodiment, the compositon comprises limonene, linalool, pinene, humulene, caryophyllene and combinations thereof. According to an embodiment, the compositon comprises limonene, linalool, eucalyptol and combinations thereof. According to various embodiments said primary composition comprises terpineol, citronellol, caryophyllene, geraniol, menthol, iso-menthone, germacrene, pinene, citronellol, cardinol, neral, geranial, myrcene, terpinene, menth-2-en-1-ol, humulene, bisabolol, borneol, nerolidol or fenchol. According to an embodiment, the composition includes pinene, terpineol, bisabolol, caryophyllene, myrcene and/or limonene, and pinene, terpineol, bisabolol, caryophyllene, myrcene and/or limonene forms less than 5% by weight of the total terpene content.

According to an embodiment said therapeutic effect treats difficulty in concentrating, brain fog and/or memory loss and said compositon comprises eucalyptol, pinene, carene, limonene and/or pulegone. According to an embodiment, the composition comprises at least 2 of these terpenes, at least 3, at least 4, or all 5 of the terpenes. According to an embodiment, the compositon comprises eucalyptol, pinene, limonene, carene and combinations thereof. According to an embodiment, the compositon comprises eucalyptol, pinene, limonene and combinations thereof. According to various embodiments said composition comprises carene, pulegone, terpineol, linalool, caryophyllene and/or cymene.

According to an embodiment said therapeutic effect treats general discomfort and said compositon comprises limonene, linalool, humulene, terpinene and/or caryophyllene. According to an embodiment, the composition comprises at least 2 of these terpenes, at least 3, at least 4, or all 5 of the terpenes. According to an embodiment, the compositon comprises linalool, humulene, terpinene, limonene and combinations thereof. According to an embodiment, the compositon comprises linalool, humulene, terpinene and combinations thereof. According to various embodiments said composition comprises limonene, borneol and/or caryophyllene. According to an embodiment, the composition includes myrcene, pinene and/or bisabolol, and myrcene, pinene and/or bisabolol forms less than 5% by weight of the total terpene content.

According to an embodiment said therapeutic effect treats muscle tension and/or myalgia and said compositon comprises myrcene, pinene, nerolidol, linalool and/or humulene. According to an embodiment, the composition comprises at least 2 of these terpenes, at least 3, at least 4, or all 5 of the terpenes. According to an embodiment, the compositon comprises myrcene, pinene, nerolidol and combinations thereof. According to an embodiment, the compositon comprises myrcene, pinene, nerolidol, linalool and combinations thereof. According to various embodiments said primary terpene comprises linalool, humulene, caryophyllene, limonene and/or bisabolol.

According to an embodiment said therapeutic effect treats fatigue and/or intermittent dizziness and said compositon comprises pinene, limonene, terpinene, eucalyptol, myrcene and/or caryophyllene. According to an embodiment, the composition comprises at least 2 of these terpenes, at least 3, at least 4, or all 5 of the terpenes. According to an embodiment, the compositon comprises pinene, limonene, eucalyptol, caryophyllene and combinations thereof. According to an embodiment, the compositon comprises pinene, limonene, eucalyptol and combinations thereof. According to various embodiments said composition comprises terpinene, myrcene, terpineol, linalool and/or caryophyllene.

According to an embodiment said therapeutic effect treats menopausal skin, itchy skin, age-related wrinkles and/or stretch marks and said compositon comprises linalool, terpineol, geraniol, citronellol and/or farnesol. According to an embodiment, the composition comprises at least 2 of these terpenes, at least 3, at least 4, or all 5 of the terpenes. According to an embodiment, the compositon comprises linalool, terpineol, geraniol, farnesol and combinations thereof. According to an embodiment, the compositon comprises linalool, terpineol, geraniol and combinations thereof. According to various embodiments said composition comprises limonene, nerolidol, pinene myrcene, nerol, citronellol, farnesol and/or carotol.

According to an embodiment said therapeutic effect treats headache and/or migraine and said compositon comprises myrcene, linalool, caryophyllene, limonene and/or nerolidol. According to an embodiment, the composition comprises at least 2 of these terpenes, at least 3, at least 4, or all 5 of the terpenes. According to an embodiment, the compositon comprises myrcene, linalool, caryophyllene, limonene and combinations thereof. According to an embodiment, the compositon comprises myrcene, linalool, caryophyllene and combinations thereof. According to various embodiments said composition comprises eucalyptol, limonene, nerolidol, fenchol, humulene, guaiol, linalool, limonene, borneol, pinene, terpinene, terpineol and/or bisabolol. According to an embodiment, the composition includes bisabolol, terpinene, humulene and/or linalool wherein bisabolol, terpinene, humulene and/or linalool forms less than 5% by weight of the total terpene content.

According to an embodiment said therapeutic effect treats weight gain and said compositon comprises comprising eucalyptol, caryophyllene, limonene, humulene and/or phytol. According to an embodiment, the composition comprises at least 2 of these terpenes, at least 3, at least 4, or all 5 of the terpenes. According to an embodiment, the compositon comprises eucalyptol, limonene, humulene, caryophyllene and combinations thereof. According to an embodiment, the compositon comprises eucalyptol, limonene, humulene and combinations thereof. According to various embodiments said composition comprises caryophyllene and/or phytol.

According to an embodiment said therapeutic effect treats digestive problems, intestinal disorders, abdominal pain (colic), nausea, constipation and/or diarrhea and said compositon comprises limonene, caryophyllene, sabinene, myrcene and/or linalool. According to an embodiment, the composition comprises at least 2 of these terpenes, at least 3, at least 4, or all 5 of the terpenes. According to an embodiment, the compositon comprises limonene, sabinene, myrcene, caryophyllene and combinations thereof. According to an embodiment, the compositon comprises limonene, sabinene, myrcene and combinations thereof. According to various embodiments said composition comprises cymene, terpinene, caryophyllene, citronellol, linalool, germacrene, geraniol, terpineol and/or pinene. According to an embodiment, the composition includes pinene, bisabolol, limonene and/or terpineol, wherein pinene, bisabolol, limonene and/or terpineol forms less than 5% by weight of the total terpene content

According to an embodiment said therapeutic effect treats breast pain and/or Mastalgia and said compositon comprises pinene, myrcene, linalool, eucalyptol and/or geraniol. According to an embodiment, the composition comprises at least 2 of these terpenes, at least 3, at least 4, or all 5 of the terpenes. According to an embodiment, the compositon comprises pinene, linalool, eucalyptol, myrcene and combinations thereof. According to an embodiment, the compositon comprises pinene, linalool, eucalyptol and combinations thereof. According to various embodiments said composition comprises limonene, caryophyllene, myrcene, geraniol, terpineol and/or sabinene.

According to an embodiment said therapeutic effect treats dysmenorrhea, pelvic pain, spasms, cramps, labor contraction and/or endometriosis and said compositon comprises myrcene, linalool, caryophyllene, eucalyptol and/or nerolidol. According to an embodiment, the composition comprises at least 2 of these terpenes, at least 3, at least 4, or all 5 of the terpenes. According to an embodiment, the compositon comprises linalool, eucalyptol, nerolidol, myrecene and combinations thereof. According to an embodiment, the compositon comprises linalool, eucalyptol, nerolidol and combinations thereof. According to an embodiment, the compositon comprises linalool, myrcene, nerolidol and combinations thereof. According to various embodiments said composition comprises myrcene, caryophyllene, limonene, nerolidol, sabinene, humulene, terpineol, citronellol, geraniol, pinene, limonene, camphene, terpinene, borneol, camphor, menthone, iso-menthone, carvacrol, thymol, carene, nerol, farnesol and/or menth-2-en-ol.

According to an embodiment said therapeutic effect treats arthralgia and/or arthritis and said compositon comprises myrcene, linalool, pinene, nerolidol and/or sabinene. According to an embodiment, the composition comprises at least 2 of these terpenes, at least 3, at least 4, or all 5 of the terpenes. According to an embodiment, the compositon comprises myrcene, pinene, sabinene, nerolidol and combinations thereof. According to an embodiment, the compositon comprises myrcene, pinene, sabinene, terpineol and combinations thereof. According to various embodiments said composition comprises linalool, guaiol, caryophyllene, eucalyptol, nerolidol, limonene, terpinene, menth-2-en-1-ol, geraniol, guaiol, humulene, bisabolol, borneol, fenchol and/or terpineol. According to an embodiment, the composition optionally includes humulene, pinene, bisabolol, borneol, fenchol, guaiol, linalool, ocimene, terpineol, terpinene and/or caryophyllene, wherein humulene, pinene, bisabolol, borneol, fenchol, guaiol, linalool, ocimene, terpineol, terpinene and/or caryophyllene forms less than 5% by weight of the total terpene content.

According to an embodiment said therapeutic effect treats hot flashes, facial flushing, night sweat, irritability, palpitation and/or tachycardia and said compositon comprises linalool, terpineol, geraniol, citronellol and/or eucalyptol. According to an embodiment, the composition comprises at least 2 of these terpenes, at least 3, at least 4, or all 5 of the terpenes. According to an embodiment, the compositon comprises linalool, pinene, eucalyptol and combinations thereof. According to an embodiment, the compositon comprises linalool, geraniol, limonene, eucalyptol and combinations thereof. According to an embodiment, the compositon comprises linalool, geraniol, eucalyptol and combinations thereof. According to various embodiments said composition comprises caryophyllene, myrcene, humulene, terpineol, geraniol, pinene, carene, citronellol, limonene, sabinene, myrtenol, champhene, borneol and/or terpinene.

According to an embodiment said therapeutic effect treats inflammation, oxidative stress and/or paresthesia and said compositon comprises caryophyllene, pinene, myrcene, eucalyptol and/or linalool. According to an embodiment, the composition comprises at least 2 of these terpenes, at least 3, at least 4, or all 5 of the terpenes. According to an embodiment, the compositon comprises caryophyllene, pinene, eucalyptol, myrcene and combinations thereof. According to an embodiment, the compositon comprises caryophyllene, pinene, eucalyptol and combinations thereof. According to various embodiments said composition comprises myrcene, limonene, humulene, citronellol, linalool, beta amyrin, cycloartenol, terpinene, terpineol, geraniol, carvacrol, thymol, cardinol, terpinene, menth-2-en-1-ol, carotol, humulene and/or nerolidol. According to an embodiment, the composition optionally includes caryophyllene, wherein caryophyllene forms less than 5% by weight of the total terpene content.

According to an embodiment said therapeutic effect treats cardiovascular disease, palpitation and tachycardia and said compositon comprises linalool, camphene, limonene, pinene and/or borneol. According to an embodiment, the composition comprises at least 2 of these terpenes, at least 3, at least 4, or all 5 of the terpenes. According to an embodiment, the compositon comprises linalool, limonene, pinene, borneol and combinations thereof. According to an embodiment, the compositon comprises linalool, limonene, pinene and combinations thereof. According to various embodiments said composition comprises camphene and/or borneol.

According to an embodiment said therapeutic effect treats urine incontinence and/or lower urinary tract disorders and said compositon comprises linalool, pinene, geraniol, caryophyllene and/or eucalyptol. According to an embodiment, the composition comprises at least 2 of these terpenes, at least 3, at least 4, or all 5 of the terpenes. According to an embodiment, the compositon comprises linalool, geraniol, pinene, caryophyllene and combinations thereof. According to an embodiment, the compositon comprises linalool, geraniol, pinene and combinations thereof. According to various embodiments said composition comprises terpineol, carene, thujone, camphor, viridiflorol, caryophyllene, germacrene, cirtonellol, eucalyptol, myrtenol, sanatalol, sabinene and/or limonene.

According to an embodiment said therapeutic effect treats vaginal dryness and said compositon comprises linalool, terpineol, geraniol, pinene and/or eucalyptol. According to an embodiment, the composition comprises at least 2 of these terpenes, at least 3, at least 4, or all 5 of the terpenes. According to an embodiment, the compositon comprises linalool, geraniol, pinene, terpineol and combinations thereof. According to an embodiment, the compositon comprises linalool, geraniol, pinene and combinations thereof. According to various embodiments said primary terpene myrcene, humulene, caryophyllene, terpineol, germacrene, cirtonellol, limonene, myrcene, terpinene, menth-2-en-1-ol, eucalyptol and/or myrtenol.

According to an embodiment said therapeutic effect treats female urogenital and/or reproductive system infection and said compositon comprises pinene, limonene, caryophyllene oxide, terpinene and/or geraniol. According to an embodiment, the composition comprises at least 2 of these terpenes, at least 3, at least 4, or all 5 of the terpenes. According to an embodiment, the compositon comprises caryophyllene oxide, pinene, limonene, geraniol and combinations thereof. According to an embodiment, the compositon comprises caryophyllene oxide, pinene, limonene and combinations thereof. According to various embodiments said composition comprises cymene, ocimene, nerolidol, eucalyptol, terpinene, terpineol, camphor, linalool, sabinene, thujone, camphor, viridiflorol, menthol, iso-menthone, caryophyllene, germacrene, geraniol, nerol, neral, geranial, citronellol, carvacol, thymol, carene, myrtenol, carene, myrcene and/or menth-2-en-1-ol. According to an embodiment the therapeutic effect treats human papillomavirus (HPV).

According to an embodiment said therapeutic effect treats fibroid, myomas and/or cysts in the female genital system and said compositon comprises linalool, terpineol, limonene, pinene and/or terpinene. According to an embodiment, the composition comprises at least 2 of these terpenes, at least 3, at least 4, or all 5 of the terpenes. According to an embodiment, the compositon comprises limonene, pinene, terpinene, linalool and combinations thereof. According to an embodiment, the compositon comprises limonene, pinene, terpinene and combinations thereof. According to various embodiments said composition comprises linalool, terpineol, caryophyllene, caryophyllene oxide and/or citronellol.

According to an embodiment said therapeutic effect treats cystitis, urethritis and/or pyelonephritis and said compositon comprises thujone, pinene, limonene, eucalyptol and/or linalool. According to an embodiment, the composition comprises at least 2 of these terpenes, at least 3, at least 4, or all 5 of the terpenes. According to an embodiment, the compositon comprises limonene, eucalyptol, linalool, pinene and combinations thereof. According to an embodiment, the compositon comprises limonene, eucalyptol, linalool and combinations thereof. According to an embodiment, the compositon comprises limonene, pinene, linalool and combinations thereof. According to various embodiments said composition comprises thujone, pinene, cymene, caryophyllene, caryophyllene oxide, terpineol, sabinene and/or cardinol.

According to an embodiment said therapeutic effect treats libido and/or sexual disfunction and said compositon comprises limonene, linalool, citronellol, geraniol and/or eucalyptol. According to an embodiment, the composition comprises at least 2 of these terpenes, at least 3, at least 4, or all 5 of the terpenes. According to an embodiment, the compositon comprises limonene, linalool, ecualyptol, geraniol and combinations thereof. According to an embodiment, the compositon comprises limonene, linalool, ecualyptol and combinations thereof. According to an embodiment, the compositon comprises citronellol, linalool, ecualyptol and combinations thereof. According to various embodiments said composition comprises terpineol, geraniol, citronellol, farnesol and/or pinene.

According to an embodiment said therapeutic effect treats hair loss and/or thinning and said compositon comprises linalool, eucalyptol, geraniol, terpineol and/or pinene. According to an embodiment, the composition comprises at least 2 of these terpenes, at least 3, at least 4, or all 5 of the terpenes. According to an embodiment, the compositon comprises geraniol, eucalyptol, terpineol, pinene and combinations thereof. According to an embodiment, the compositon comprises geraniol, eucalyptol, terpineol and combinations thereof. According to various embodiments said composition comprises linalool, borneol, camphor and/or pinene.

According to an embodiment said therapeutic effect treats brittle nails and said compositon comprises linalool, eucalyptol, geraniol, pinene and/or limonene. According to an embodiment, the composition comprises at least 2 of these terpenes, at least 3, at least 4, or all 5 of the terpenes. According to an embodiment, the compositon comprises linalool, eucalyptol, limonene, geraniol and combinations thereof. According to an embodiment, the compositon comprises linalool, eucalyptol, limonene and combinations thereof. According to various embodiments said composition comprises geraniol and/or pinene.

According to an embodiment said therapeutic effect treats irregular body odor and said compositon comprises linalool, terpineol, geraniol, and/or eucalyptol. According to an embodiment, the composition comprises at least 2 of these terpenes, at least 3, or all 4 of the terpenes. According to an embodiment, the compositon comprises linalool, terpineol, geraniol and combinations thereof. According to an embodiment, the compositon comprises linalool, terpineol, geraniol and combinations thereof.

According to an embodiment said therapeutic effect treats Polycystic Ovary Syndrome (POCS), amenorrhea, oligomenorrhea, hypermenorrhea, metrorrhagia and said compositon comprises linalool, caryophyllene, geraniol, pinene and/or eucalyptol. According to an embodiment, the composition comprises at least 2 of these terpenes, at least 3, at least 4, or all 5 of the terpenes. According to an embodiment, the compositon comprises linalool, eucalyptol, geraniol and combinations thereof. According to an embodiment, the compositon comprises linalool, eucalyptol, caryophyllene, pinene and combinations thereof. According to various embodiments said composition comprises humulene, terpineol, caryophyllene, caryophyllene oxide, terpinene, citronellol, thujone, camphor, viridiflorol, pinene, camphene, sabinene, myrcene, carene, carotol, limonene, sabinene, cardinal and/or pinene.

According to an embodiment said therapeutic effect treats premenstrual syndrome (PMS), premenstrual tension, and/or premenstrual dysphoric disorder (PMDD) and said compositon comprises limonene, linalool, eucalyptol, geraniol and/or caryophyllene. According to an embodiment, the composition comprises at least 2 of these terpenes, at least 3, at least 4, or all 5 of the terpenes. According to an embodiment, the compositon comprises linalool, eucalyptol, geraniol, limonene and combinations thereof. According to an embodiment, the compositon comprises linalool, caryophyllene, limonene and combinations thereof. According to an embodiment, the compositon comprises linalool, caryophyllene, limonene, eucalyptol, geraniol and combinations thereof. According to various embodiments said composition comprises limonene, nerolidol, sabinene, pinene, caryophyllene, terpineol, thujone, camphor, viridiflorol, champhene, myrcene, caryophyllene, terpinene, carene, cirtonellol, carotol, neral and/or geranial.

According to an embodiment said therapeutic effect treats hormonal disfunction and/or imbalance and/or fertility and said compositon comprises linalool, geraniol, pinene, eucalyptol and/or limonene. According to an embodiment, the composition comprises at least 2 of these terpenes, at least 3, at least 4, or all 5 of the terpenes. According to an embodiment, the compositon comprises linalool, eucalyptol, geraniol and combinations thereof. According to an embodiment, the compositon comprises linalool, eucalyptol, geraniol, terpineol and combinations thereof. According to an embodiment, the compositon comprises linalool, eucalyptol, geraniol, pinene, caryophyllene and combinations thereof. According to various embodiments said composition comprises terpineol, geraniol, thujone, camphor, viridiflorol, borneol, champhene, pinene, caryophyllene, germacrene, pinene, carene, cirtonellol, nerol, farnesol, limonene, myrcene, terpinene, menth-2-en-1-ol, carotol, sabinene, neral and/or geranial.

According to an embodiment said therapeutic effect treats pre-eclampsia toxemia and said compositon comprises limonene, neral, caryophyllene, linalool and/or myrcene. According to an embodiment, the composition comprises at least 2 of these terpenes, at least 3, at least 4, or all 5 of the terpenes. According to an embodiment, the compositon comprises linalool, myrcene, limonene and combinations thereof. According to an embodiment, the compositon comprises linalool, myrcene, limonene, caryophyllene and combinations thereof. According to various embodiments said composition comprises neral, geranial, caryophyllene, nerolidol, fenchol, humulene, guaiol, borneol, cymene and/or terpinene.

According to an embodiment said therapeutic effect treats conditions and/or symptoms associated with female hormonal disfunction and/or imbalance and said compositon comprises linalool, terpineol, geraniol, pinene and/or eucalyptol. According to an embodiment, the composition comprises at least 2 of these terpenes, at least 3, at least 4, or all 5 of the terpenes. According to an embodiment, the compositon comprises geraniol, linalool, eucalyptol and combinations thereof. According to an embodiment, the compositon comprises terpineol, linalool, caryophyllene, pinene and combinations thereof. According to an embodiment, the compositon comprises geraniol, linalool, eucalyptol, caryophyllene, terpineol, pinene, limonene and combinations thereof. According to various embodiments said composition comprises terpineol, geraniol, thujone, camphor, viridiflorol, borneol, champhene, pinene, eucalyptol, caryophyllene, germacrene, pinene, carene, cirtonellol, nerol, farnesol, limonene, myrcene, terpinene, menth-2-en-1-ol, carotol, sabinene, neral, geranial, nerolidol, terpineol and/or careen.

According to an embodiment said therapeutic effect treats perimenopause and/or menopause and said compositon comprises caryophyllene, carene, linalool, myrcene and/or limonene. According to an embodiment, the composition comprises at least 2 of these terpenes, at least 3, at least 4, or all 5 of the terpenes. According to an embodiment, the compositon comprises caryophyllene, linalool, carene, limonene and combinations thereof. According to an embodiment, the compositon comprises caryophyllene, linalool, carene and combinations thereof. According to an embodiment, the compositon comprises caryophyllene, linalool, myrcene and combinations thereof. According to an embodiment, the compositon comprises nerolidol, limonene, linalool and combinations thereof. According to an embodiment, the compositon comprises caryophyllene, linalool, carene, nerolidol, limonene and combinations thereof. According to various embodiments said composition comprises caryophyllene, caryophyllene oxide, carene, myrcene, terpinene, terpineol, citronellol, eucalyptol, nerolidol, limonene, geraniol, menthol, iso-menthone, germacrene, pinene, cardinol, neral, geranial, menth-2-en-1-ol, pulegone, cymene, fenchol, humulene, guaiol, borneol, carene, sabinene, myrtenol, camphene, nerol, farnesol and/or carotol.

According to an embodiment said therapeutic effect treats pain and/or cramps and said compositon comprises myrcene, linalool, eucalyptol, nerolidol and/or limonene. According to an embodiment, the composition comprises at least 2 of these terpenes, at least 3, at least 4, or all 5 of the terpenes. According to an embodiment, the compositon comprises limonene, linalool, eucalyptol and combinations thereof. According to an embodiment, the compositon comprises limonene, linalool, eucalyptol, myrcene and combinations thereof. According to various embodiments said composition comprises myrcene, caryophyllene, nerolidol, sabinene, humulene, terpineol, citronellol, geraniol, pinene, camphene, terpinene, borneol, camphor, menthone, iso-menthone, carvacrol, thymol, carene, nerol, farnesol, menth-2-en-1-ol and/or guaiol.

According to an embodiment said therapeutic effect treats female urogenital and/or reproductive system infections and/or disorders and said compositon comprises linalool, pinene, caryophyllene oxide, borneol and/or eucalyptol. According to an embodiment, the composition comprises at least 2 of these terpenes, at least 3, at least 4, or all 5 of the terpenes. According to an embodiment, the compositon comprises linalool, pinene, caryophyllene oxide and combinations thereof. According to an embodiment, the compositon comprises geraniol, linalool, pinene, caryophyllene oxide and combinations thereof. According to various embodiments said composition comprises myrcene, humulene, caryophyllene, terpineol, geraniol, germacrene, cirtonellol, limonene, myrcene, terpinene, menth-2-en-1-ol, borneol, eucalyptol, myrtenol, cymene, ocimene, nerolidol, camphor, sabinene, thujone, camphor, viridiflorol, menthol, iso-menthone, caryophyllene, germacrene, geraniol, nerol, neral, geranial, citronellol, carvacol, thymol, carene, myrtenol, carene and/or thujone.

According to an embodiment, the method for treating a patient comprises administering to said patient a product selected from the group consisting of tablets, gel capsules, medical patches, topicals, creams, varnishes, sublingual oils, sprays, edibles, suppositories including tampons and/or rectal candles, liquid nasal preparation, preparations containing micro and nano-emulsions, preparations containing micro and nano-particles, cigarettes, vaporizer liquids and combinations thereof, containing the administered composition. According to an embodiment, the product comprising tablets containing the composition. According to an embodiment, the product comprising gel capsules containing the composition. According to an embodiment, the product comprising medical patches containing the composition. According to an embodiment, the product comprising topicals containing the composition. According to an embodiment, the product comprising creams containing the composition. According to an embodiment, the product comprising varnishes containing the composition. According to an embodiment, the product comprising sublingual oils containing the composition. According to an embodiment, the product comprising edibles containing the composition. According to an embodiment, the product comprising tampons containing the composition. A According to an embodiment, the product comprising rectal candles containing the composition. According to an embodiment, the product comprising cigarettes containing the composition. According to an embodiment, the product comprising vaporizer liquids containing the composition.

### Examples 1-100

The Table presents examples of compositions as described herein.

| | | | | Content | | | |
|---|---|---|---|---|---|---|---|
| | | | | (% by weight) | | | |
| | | Canna- | Primary | Canna- | Primary | | Therapeutic |
| | Form | binoid | terpene | binoid | terpene | Additive | effect for: |
| 1 | Cigarette | CBD | Caryophyllene | 3-25 | 1-10 | Biochanin A | Osteoporosis |
| 2 | Cigarette | THC | Terpineol | 3-25 | 1-10 | | Sleep disorder |
| 3 | Cigarette | CBD | Linalool | 3-25 | 1-10 | | cramps |
| 4 | Cigarette | CBD | Caryophyllene | 3-25 | 1-10 | Extract of | Urine |
| | | | | | | barosma | incontinence |
| | | | | | | betulina | |
| 5 | Cigarette | THC | Pinene | 3-25 | 1-10 | | Muscle |
| | | and/or | | | | | tension |
| | | CBD | | | | | |
| 6 | Cigarette | THC | Myrcene | 3-25 | 1-10 | | Myalgia |
| | | and/or | | | | | |
| | | CBD | | | | | |
| 7 | Cigarette | THC | Citronellol | 3-25 | 1-10 | | Sleep disorder |
| 8 | Cigarette | CBD | Pinene | 3-25 | 1-10 | Extract of | Brain fog |
| | | | | | | ginkgo | |
| | | | | | | biloba | |
| 9 | Cigarette | CBD | Limonene | 3-25 | 1-10 | | Anxiety |
| 10 | Oil | CBD | Linalool | 3-25 | 1-10 | | POCS |
| 11 | Cigarette | THC | Limonene | 3-25 | 1-10 | Genistein | Hot flashes |
| | | and/or | | | | | |
| | | CBD | | | | | |
| 12 | Cigarette | CBD | Eucalyptol | 3-25 | 1-10 | | Anxiety |
| 13 | Cigarette | CBD | Humulene | 3-25 | 1-10 | | Mood swings |
| 14 | Cigarette | THC | Eucalyptol | 3-25 | 1-10 | Extract of | Sleep disorder |
| | | | | | | melissa | |
| | | | | | | officinalis | |
| 15 | Cigarette | THC | Pinene | 3-25 | 1-10 | | Muscle |
| | | and/or | | | | | tension |
| | | CBD | | | | | |
| 16 | Grinded | CBD | caryophyllene | 3-25 | 1-10 | Extract of | Osteoporosis |
| | bud | | | | | angelica | |
| | | | | | | sinensis | |
| 17 | Oil [1] | CBD | Caryophyllene | 3-40 | 1-20 | | Osteoporosis |
| 18 | Oil | THC | Myrcene | 3-40 | 1-20 | | Muscle |
| | | and/or | | | | | tension |
| | | CBD | | | | | |
| 19 | Oil | THC | Terpineol | 3-40 | 1-20 | | Sleep disorder |
| 20 | Oil | CBG | Linalool | 3-40 | 1-20 | Extract of | Stress |
| | | | | | | bacopa | |
| | | | | | | monnieri | |
| 21 | Oil | CBD | Eucalyptol | 3-40 | 1-20 | | irritability |
| 22 | Oil | THC | Nerolidol | 3-40 | 1-20 | | Muscle |
| | | and/or | | | | | tension |
| | | CBD | | | | | |
| 23 | Oil | CBC | Terpineol + | 3-40 | 1-10 | | Anxiety |
| | | | Citronellol + | | | | |
| | | | Linalool | | | | |
| 24 | Oil | CBD | Citronellol | 3-40 | 1-10 | | PMS |
| 25 | Topical | THC | Pinene | 3-40 | 1-20 | Extract of | Muscle |
| | | and/or | | | | boswellia | tension |
| | | CBD | | | | spp | |
| 26 | Oil | CBD | Eucalyptol | 3-40 | 1-10 | | Anxiety |
| 27 | Oil | CBN | Linalool | 3-40 | 1-10 | Linalyl | Sleep |
| | | | | | | acetate | disorder |
| 28 | Oil | CBD | Terpineol | 3-40 | 1-20 | | restless |
| 29 | Oil | THC | Eucalyptol | 3-40 | 1-20 | | Sleep |
| | | | | | | | disorder |
| 30 | Oil | CBD | Geraniol | 3-40 | 1-20 | Daidzein | Hot flashes |
| 31 | Oil | CBG | Limonene | 3-40 | 1-20 | | Anxiety |
| 32 | Oil | THC | Terpineol + | 3-40 | 1-10 | | Sleep |
| | | | Citronellol + | | | | disorder |
| | | | Linalool | | | | |
| 33 | Tablets | THC | Linalool | 1-10 | 1-10 | | Sleep |
| | | | | | | | disorder |
| 34 | Tablets | CBD | Caryophyllene | 1-10 | 1-10 | Glycitein | Osteoporosis |
| 35 | Tablets | CBD | Linalool | 1-10 | 1-10 | | Anxiety |
| 36 | Tablets | THC | Citronellol | 1-10 | 1-20 | | Sleep |
| | | | | | | | disorder |
| 37 | Tablets | CBC | Eucalyptol | 1-10 | 1-20 | Extract of | cystitis |
| | | | | | | althea | |
| | | | | | | officinalis | |
| 38 | Tablets | CBD | Pinene | 1-10 | 1-10 | | Memory |
| | | | | | | | loss |
| 39 | Tablets | THC | Terpineol | 1-10 | 1-10 | | Sleep |
| | | | | | | | disorder |
| 40 | Tablets | CBD | Pinene | 1-10 | 1-10 | Extract of | Arthritis |
| | | | | | | salix alba | |
| 41 | Tablets | THC | Myrcene | 1-10 | 1-10 | | Muscle |
| | | and/or | | | | | tension |
| | | CBD | | | | | |
| 42 | Tablets | THC | Eucalyptol | 1-10 | 1-10 | Formonene | Sleep |
| | | | | | | tin | disorder |
| 43 | Tablets | THC | Nerolidol | 1-10 | 1-10 | | Muscle |
| | | and/or | | | | | tension |
| | | CBD | | | | | |
| 44 | Tablets | CBD | Limonene | 1-10 | 1-10 | | Anxiety |
| 45 | Tablets | CBD | Eucalyptol | 1-10 | 1-10 | Avena sativa | Mood |
| | | | | | | | swings |
| 46 | Tablets | THC | Pinene | 1-10 | 1-10 | | Muscle |
| | | and/or | | | | | tension |
| | | CBD | | | | | |
| 47 | Topical | THC | Myrcene | 3-25 | 1-10 | | Headaches |
| | | and/or | | | | | |
| | | CBD | | | | | |
| 48 | Cigarette | CBD | Pinene | 3-25 | 1-10 | | Fatigue |
| 49 | suppositories | THC | Limonene | 3-25 | 1-10 | | Digestive |
| | | and/or | | | | | problems |
| | | CBD | | | | | |
| 50 | Cigarette | CBD | Terpinene | 3-25 | 1-10 | | Fatigue |
| 51 | Cigarette | THC | terpinene | 3-25 | 1-10 | Piperine | Digestive |
| | | and/or | | | | | problems |
| | | CBD | | | | | |
| 52 | Suppositories | THC | Linalool | 3-25 | 1-10 | Extract of | Dysmenorrhea |
| | | and/or | | | | cimicifuga | |
| | | CBD | | | | (actaea) | |
| | | | | | | racemosa | |
| 53 | Cigarette | CBD | Phytol | 3-25 | 1-10 | | Weight gain |
| 54 | Topical | CBD | Linalool | 3-25 | 1-10 | | Itchy skin |
| 55 | Cigarette | THC | caryophyllene | 3-25 | 1-10 | | Headaches |
| | | and/or | | | | | |
| | | CBD | | | | | |
| 56 | Cigarette | CBD | Limonene | 3-25 | 1-10 | Extract of | Weight gain |
| | | | | | | laminaria sp_{]} | |
| 57 | suppositories | THC | Cymene | 3-25 | 1-10 | | Digestive |
| | | and/or | | | | | problems |
| | | CBD | | | | | |
| 58 | Cigarette | THC | Caryophyllene | 3-25 | 1-10 | | Endometriosis |
| | | and/or | | | | | |
| | | CBD | | | | | |
| 59 | Cigarette | THC | Pinene | 3-25 | 1-10 | | Hormonal |
| | | and/or | | | | | imbalance |
| | | CBD | | | | | |
| 6o | Topical | THC | Limonene | 3-25 | 1-10 | | PMDD |
| | | and/or | | | | | |
| | | CBD | | | | | |
| 61 | Cigarette | THC | Myrcene | 3-25 | 1-10 | Extract of | Dysmenorrhea |
| | | and/or | | | | vitex agnus | |
| | | CBD | | | | castus | |
| 62 | Oil | CBD | Linalool | 3-40 | 1-20 | Extract of | stretch marks |
| | | | | | | glycyrrhiz | |
| | | | | | | a glabra | |
| 63 | Oil | CBD | Terpinene | 3-40 | 1-20 | | Fatigue |
| 64 | Oil | THC | Caryophyllene | 3-40 | 1-20 | | Headaches |
| | | and/or | | | | | |
| | | CBD | | | | | |
| 65 | Oil | CBD | Eucalyptol | 3-40 | 1-20 | Biochanin A | sexual |
| | | | | | | | disfunction |
| 66 | Oil | THC | Myrcene | 3-40 | 1-20 | | Headaches |
| | | and/or | | | | | |
| | | CBD | | | | | |
| 67 | Oil | THC | Pinene | 3-40 | 1-20 | | Breast pain |
| | | and/or | | | | | |
| | | CBD | | | | | |
| 69 | Oil | CBD | Pinene | 3-40 | 1-20 | | Fatigue |
| 69 | Oil | THC | Linalool | 3-40 | 1-20 | Formononeti | Infertility |
| | | and/or | | | | | |
| | | CBD | | | | | |
| 70 | Oil | THC | Eucalyptol | 3-40 | 1-20 | Extract of | Headaches |
| | | and/or | | | | corydalis | |
| | | CBD | | | | yanhusuo | |
| 71 | Oil | THC | Limonene | 3-40 | 1-20 | | Abdominal |
| | | and/or | | | | | pain |
| | | CBD | | | | | |
| 72 | Oil | CBD | Phytol | 3-40 | 1-20 | | Weight gain |
| 73 | suppositories | THC | Linalool | 3-40 | 1-20 | Genistein | Endometriosis |
| | | and/or | | | | | |
| | | CBD | | | | | |
| 74 | Oil | THC | Cymene | 3-40 | 1-20 | | Digestive |
| | | and/or | | | | | problem |
| | | CBD | | | | | |
| 75 | Oil | THC | Limonene | 3-40 | 1-20 | | Breast pain |
| | | and/or | | | | | |
| | | CBD | | | | | |
| 76 | suppositories | THC | Myrcene | 3-40 | 1-20 | | Dysmenorrhea |
| | | and/or | | | | | |
| | | CBD | | | | | |
| 77 | Oil | THC | Terpinene | 3-40 | 1-20 | Prenylnarin- | Vaginal |
| | | and/or | | | | genin | dryness |
| | | CBD | | | | | |
| 78 | Oil | THC | Caryophyllene | 3-40 | 1-20 | | Dysmenorrhea |
| | | and/or | | | | | |
| | | CBD | | | | | |
| 79 | suppositories | THC | Caryophyllene | 1-10 | 1-10 | | pelvic pain |
| | | and/or | | | | | |
| | | CBD | | | | | |
| 80 | Tablets | THC | Terpinene | 1-10 | 1-10 | Extract of | Urogenital |
| | | and/or | | | | coptis | infection |
| | | CBD | | | | chinensis, | |
| 81 | Tablets | CBD | Pinene | 1-10 | 1-10 | | Fatigue |
| 82 | Tablets | THC | Limonene | 1-10 | 1-10 | | Breast pain |
| | | and/or | | | | | |
| | | CBD | | | | | |
| 83 | suppositories | THC | Linalool | 1-10 | 1-10 | | pelvic pain |
| | | and/or | | | | | |
| | | CBD | | | | | |
| 84 | Tablets | CBD | Terpinene | 1-10 | 1-10 | | Fatigue |
| 85 | Tablets | THC | Pinene | 1-10 | 1-10 | | Breast pain |
| | | and/or | | | | | |
| | | CBD | | | | | |
| 86 | Tablets | THC | Myrcene | 1-10 | 1-10 | | Dysmenorrhea |
| | | and/or | | | | | |
| | | CBD | | | | | |
| 87 | Tablets | CBD | Linalool | 1-10 | 1-10 | Eugenol | POCS |
| 88 | Tablets | THC | cymene | 1-10 | 1-10 | | Intestinal |
| | | and/or | | | | | disorders |
| | | CBD | | | | | |
| 89 | Tablets | CBD | Limonene | 1-10 | 1-10 | | Weight gain |
| 90 | Tablets | THC | Myrcene | 1-10 | 1-10 | Extract of | Amenorrhea |
| | | and/or | | | | vitex agnus | |
| | | CBD | | | | castus | |
| 91 | Tablets | THC | Limonene | 1-10 | 1-10 | | Digestive |
| | | and/or | | | | | problems |
| | | CBD | | | | | |
| 92 | Tablets | CBD | Phytol | 1-10 | 1-10 | | Weight gain |
| 93 | Tablets | THC | Caryophyllene | 1-10 | 1-10 | Extract of | Myoma |
| | | and/or | | | | angellica | |
| | | CBD | | | | sinesis | |
| 94 | Tablets | THC | Eucalyptol | 1-10 | 1-10 | | Headaches |
| | | and/or | | | | | |
| | | CBD | | | | | |
| 95 | Tablets | CBD | Eucalyptol | 1-10 | 1-10 | Extract of | PMS |
| | | | | | | angelica | |
| | | | | | | sinensis | |
| 96 | Tablets | THC | Caryophyllene | 1-10 | 1-10 | | Headaches |
| | | and/or | | | | | |
| | | CBD | | | | | |
| 97 | suppositories | CBD | Terpineol | 1-10 | 1-10 | | cystitis |
| 98 | Grinded | THC | Caryophyllene | 1-10 | 1-10 | | Irregular |
| | bud | | | | | | periods |
| 99 | Oil | CBD | Humulene | 1-10 | 1-10 | | POCS |
| 100 | Tables | CBD | Limonene | 1-10 | 1-10 | Extract of | PMDD |
| | | | | | | cimicifuga | |
| | | | | | | (actaea) | |
| | | | | | | racemosa | |

[1] Oil herein refers to a composition containing an extract of a cannabis plant material.

### Example 101: Treating a Menopause Woman with Symptoms of Headaches and Migraines

A menopausal woman at the age of 49 with symptoms of headaches and migraines is treated by administering ground cannabis in a vaporizer. The ground cannabis contains 16% THC, 0.1% CBD and a primary terpene containing myrcene. Administrating involves 10 puffs per dose, twice a day. Each administrated dose contains 100 mg of THC, 0.6 mg of CBD and 12 mg myrcene.

### Example 102: Treating a Menopause Woman with Symptoms of Anxiety

A menopausal woman at the age of 53 with symptoms of stress and anxiety is treated by administering cannabis cigarettes. The cigarettes contain 0.5% THC, 15% CBD and a primary terpene containing linalool. Administrating involves 5 puffs per dose, 3 times per day. Each administrated dose contains 2 mg of THC, 50 mg of CBD and 8 mg linalool.

### Example 103: Treating a Perimenopause Woman with Symptoms of Depression

A perimenopausal woman at the age of 42 with symptoms of depression is treated by administering cannabis oil sublingually. The oil contains 5.5% THC, 9% CBD and a primary terpene containing limonene. Administrating involves 5 droplets per dose, 3 times per day. Each administrated dose contains 16 mg of THC, 26 mg of CBD and 10 mg limonene.

### Example 104: Treating a Woman with Symptoms of Cramps and Pelvic Pain

A woman at the age of 25 with symptoms of cramps and pelvic pain is treated by administering ground cannabis in a vaporizer. The ground cannabis contains 0.4% THC, 12% CBD and a primary terpene containing caryophyllene. Administrating involves 10 puffs per dose, twice a day. Each administrated dose contains 2.5 mg of THC, 73 mg of CBD and 15 mg caryophyllene.

### Example 105: Treating a Woman with Endometriosis

A woman at the age of 30 diagnosed with endometriosis is treated by administering cannabis oil sublingually. The cannabis oil contains 0.1% THC, 23% CBD and a primary terpene containing nerolidol. Administrating involves 1 droplet per dose, twice a day. Each administrated dose contains 0.04 mg of THC, 9.2 mg of CBD and 2.5 mg nerolidol.

### Example 106: Treating a Woman with Arthritis

A woman at the age of 58 diagnosed with arthritis is treated by administering ground cannabis in a vaporizer. The ground cannabis contains 10% THC, 1% CBD and a primary terpene containing sabinene. Administrating involves 5 puffs per dose, 3 times per day. Each administrated dose contains 50 mg of THC, 5 mg of CBD and 12 mg sabinene.

### Example 107: Treating a Menopause Woman with Symptoms of Insomnia

A menopause woman at the age of 48 with symptoms of insomnia is treated by administering cannabis cigarettes. The cigarette contains 16% THC, 1% CBD and a primary terpene containing terpineol. Administrating involves 10 puffs per dose, 30 minutes before bedtime. Each administrated dose contains 100 mg of THC, 6 mg of CBD and 20 mg terpineol.

### Example 108: Treating a Woman with Dysmenorrhea

A woman at the age of 33 diagnosed with dysmenorrhea is treated by administering cannabis oil sublingually. The cannabis oil contains 2% THC, 25% CBD and a primary terpene containing myrcene. Administrating involves 1 droplet per dose, twice a day. Each administrated dose contains 0.6 mg of THC, 8 mg of CBD and 3 mg myrcene.

### Example 109: Treating a Woman with Osteoporosis

A woman at the age of 58 diagnosed with osteoporosis is treated by administering ground cannabis in a vaporizer. The ground cannabis contains 9.2% THC, 5.7% CBD and a primary terpene containing caryophyllene. Administrating involves 3 puffs per dose, 3 times per day. Each administrated dose contains 21 mg of THC, 13 mg of CBD and 12 mg caryophyllene.

### Example 110: Treating a Menopause Woman with Symptoms of Mood Swings

A menopause woman at the age of 51 with symptoms of mood swings is treated by administering tablets of cannabis extract. Each tablet contains 2% THC, 25% CBD and a primary terpene containing eucalyptol. Administrating involves 1 tablet per dose, twice a day. Each administrated dose contains 0.7 mg of THC, 9.5 mg of CBD and 3 mg eucalyptol.

### Example 111: Treating a Woman with Endometriosis

A woman at the age of 30 diagnosed with endometriosis is treated by administering cannabis oil sublingually. The cannabis oil contains 2% THC, 25% CBD and a primary terpene containing nerolidol. Administrating involves 3 droplets per dose, twice a day. Each administrated dose contains 1.8 mg of THC, 24 mg of CBD and 8 mg nerolidol.

### Example 112: Treating a Woman with Polycystic Ovary Syndrome (POCS)

A woman at the age of 28 diagnosed with POCS is treated by administering cannabis oil sublingually. The cannabis oil contains 12.5% THC, 12.5% CBD and a primary terpene containing geraniol. Administrating involves 1 droplet per dose, 3 times per day. Each administrated dose contains 5.5 mg of THC, 5.5 mg of CBD and 3 mg geraniol.

### Example 113: Treating a Woman with Premenstrual Syndrome (PMS)

A woman at the age of 19 diagnosed with premenstrual syndrome (PMS) is treated by administering cannabis oil sublingually. The cannabis oil contains 15% THC, 0.4% CBD and a primary terpene containing terpinene. Administrating involves 1 droplet per dose, twice a day. Each administrated dose contains 22 mg of THC, 0.6 mg of CBD and 4 mg terpinene.

### Example 114: Treating a Woman with an Hormonal Imbalance

A woman at the age of 30 diagnosed with hormonal imbalance is treated by administering tablets of cannabis extract. Each tablet contains 1.3% THC, 31% CBD and a primary terpene containing terpineol. Administrating involves 1 tablet per dose, twice a day. Each administrated dose contains 0.5 mg of THC, 14 mg of CBD and 2.5 mg terpineol.

### Example 115: Treating a Menopause Woman with Symptoms of Hot Flashes

A menopausal woman at the age of 48 with symptoms of hot flashes is treated by administering ground cannabis in a vaporizer. The ground cannabis contains 13.5% THC, 0.4% CBD and a primary terpene containing geraniol. Administrating involves 12 puffs per dose, twice a day. Each administrated dose contains 100 mg of THC, 3 mg of CBD and 18 mg geraniol.

### Example 116: Treating a Woman with Cystitis

A woman at the age of 22 diagnosed with cystitis is treated by administering cannabis oil sublingually. The cannabis oil contains 5.5% THC, 5.5% CBD and a primary terpene containing pinene. Administrating involves 4 droplet per dose, 3 times per day. Each administrated dose contains 9 mg of THC, 9 mg of CBD and 4 mg pinene.

### Example 117: Treating a Perimenopause Woman with Symptoms of Vaginal Dryness

A perimenopausal woman at the age of 45 with symptoms of vaginal dryness is treated by administering cannabis oil sublingually. The cannabis oil contains 2% THC, 25% CBD and a primary terpene containing geraniol. Administrating involves 3 droplets per dose, twice a day. Each administrated dose contains 1.8 mg of THC, 24 mg of CBD and 5 mg geraniol.

### Example 118: Treating a Menopause Woman with Symptoms of Irritability

A menopausal woman at the age of 49 with symptoms of irritability is treated by administering cannabis oil sublingually. The oil contains 5.5% THC, 9% CBD and a primary terpene containing limonene. Administrating involves 5 droplets per dose, 3 times per day. Each administrated dose contains 16 mg of THC, 26 mg of CBD and 10 mg limonene.

### Example 119: Treating a Perimenopause Woman with Symptoms of Urine Incontinence

A perimenopausal woman at the age of 41 with symptoms of urine incontinence is treated by administering cannabis oil sublingually. The oil contains 9.5% THC, 0.01% CBD and a primary terpene containing pinene. Administrating involves 4 droplets per dose, 3 times per day. Each administrated dose contains 27 mg of THC, 0.03 mg of CBD and 8 mg pinene.

### Example 120: Treating a Menopause Woman with Symptoms of Memory Loss

A menopausal woman at the age of 54 with symptoms of memory loss is treated by administering cannabis cigarettes. The cigarettes contains 17.5% THC, 0.05% CBD and a primary terpene containing pinene. Administrating involves 7 puffs per dose, twice a day. Each administrated dose contains 77 mg of THC, 0.2 mg of CBD and 20 mg pinene.

Thus, the scope of the invention shall include all modifications and variations that may fall within the scope of the attached claims. Other embodiments of the invention will be apparent to those skilled in the art from consideration of the specification and practice of the invention disclosed herein. It is intended that the specification and examples be considered as exemplary only, with a true scope and spirit of the invention being indicated by the

[symbol]

1. A product for treating conditions and/or symptoms associated with women health, which product comprises
(i) at least one cannabinoid in a specific amount,
(ii) a primary terpene in a specific amount,
(iii) at least 5% by weight of a non-cannabinoid, non-terpene carrier,
(iv) optionally at least three secondary terpenes,
(v) optionally at least one phytoestrogen,
and;
(a) wherein said non-cannabinoid, non-terpene carrier comprises cellulose and the terpenes to cannabinoids weight/weight ratio in said product is about 0.1 to about 1.0,
or;
(b) wherein said non-cannabinoid, non-terpene carrier comprises less than 5% by weight cellulose and the terpenes to cannabinoids weight/weight ratio in said product is about 0.05 to about 1.0,
forming a terpene-enriched product with an enhanced therapeutic effect in treating conditions and/or symptoms associated with perimenopause, menopause, female urogenital or reproductive system infections and/or disorders, menstrual cycle, hormonal deficiency and/or hormonal imbalance, compared with that of a product comprising the same cannabinoids amounts and one half the amount of said primary terpene.

2. A method of treating conditions and/or symptoms associated with perimenopause, menopause, female urogenital or reproductive system infections and/or disorders, menstrual cycle, hormonal deficiency and/or hormonal imbalance comprising administering to a subject in need a therapeutically effective amount of a composition comprising
(i) a primary terpene in a specific amount;
(ii) optionally at least three secondary terpenes;
(iii) optionally at least one cannabinoid in a specific amount; and
optionally at least one phytoestrogen.

3. The method of claim 2, wherein said product comprises
(i) said at least one cannabinoid in a specific amount;
(ii) said primary terpene in a specific amount; and further comprises
(iii) at least 5% by weight of a non-cannabinoid, non-terpene carrier,
optionally combined with at least one herbal extract.

4. The method of claim 3, wherein said phytoestrogen comprises at least one of biochanin A, formonenetin, genistein, daidzein, glycitein, prenylnaringenin, diosgenin, acetin and/or cimicifugoside.

5. The method of claim 3, wherein said non-cannabinoid, non-terpene carrier comprises cellulose and the terpenes to cannabinoids weight/weight ratio in said product is about 0.1 to about 1.0, or wherein said non-cannabinoid, non-terpene carrier comprises less than 5% by weight cellulose and the terpenes to cannabinoids weight/weight ratio in said product is about 0.05 to about 1.0.

6. The method of claim 3, wherein said conditions and/or symptoms are selected from the group consisting of hot flashes, facial flushing, night sweat, osteoporosis, fatigue, irritability, insomnia and/or sleep disorder, difficulty in concentration, depression, memory loss, brain fog, headache, migraine, anxiety, nervousness, restless, stress, mood swing, affect disorder, general discomfort, muscle tension, myalgia, intermittent dizziness, paresthesia, indigestion, digestive problems, intestinal disorders, abdominal pain, irritability, palpitations, tachycardia, nausea, constipation, diarrhea, arthralgia and/or arthritis, dysmenorrheal, pelvic pain, spasms, cramps, labor contraction, endometriosis Polycystic Ovary Syndrome (POCS), amenorrhea, oligomenorrhea, hypermenorrhea, metrorrhagia, premenstrual syndrome (PMS), premenstrual tension, premenstrual dysphoric disorder (PMDD), cold hands and feet, weight gain, mental awareness problems, sexual pain disorder, dyspareunia, breast pain and/or mastalgia, menopausal skin, inflammation, oxidative stress, cardiovascular disease, loss of pelvic muscle tone, urinary incontinence, lower urinary tract disorders, vaginal dryness, women urogenital and/or reproductive system infection, fibroid, myomas and/or cysts in the female genital system, human papillomavirus (HPV), Uterine Fibroid, cystitis, urethritis, pyelonephritis, women hormonal dysfunction and/or imbalance, irregular periods, pre-eclampsia toxemia, reduced libido, reduced sexual dysfunction, hair loss and/or thinning, age-related winkles, stretch marks, brittle nails, gam disorders, irregular body odor, conditions and/or symptoms induced by estrogen deficiency and combinations thereof.

7. The method of claim 3, wherein said product additionally comprises an additive selected from the group consisting of antioxidants, emulsifiers and texturizers vegetable oils, plant extracts, honey, sucrose, glucose and fructose, pharmaceutical excipients and combinations thereof.

8. The method of claim 3, wherein said primary terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thujone, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

9. The method of claim 3, wherein the primary terpene and the cannabinoids are present in specific amounts, and the onset time of said therapeutic effect is at least 20% shorter than that of administering a product comprising the same cannabinoids amounts and one half the amount of said primary terpene; wherein the primary terpene and the cannabinoids are present in specific amounts, and the magnitude of the therapeutic effect is at least 20% greater compared with that of administering a product comprising the same cannabinoids amounts and one half the amount of said primary terpene; wherein the primary terpene and the cannabinoids are present in specific amounts, and the duration of the therapeutic effect is at least 20% longer compared with that of administering a product comprising the same cannabinoids amounts and one half the amount of said primary terpene;
wherein the primary terpene and the cannabinoids are present in specific amounts, and the frequency of said conditions and/or symptoms is at least 20% smaller compared with that of administering a product comprising the same cannabinoids amounts and one half the amount of said primary terpene; wherein the primary terpene and the cannabinoids are present in specific amounts, and the severity of said conditions and/or symptoms is at least 20% smaller compared with that of administering a product comprising the same cannabinoids amounts and one half the amount of said primary terpene; or wherein the primary terpene and the cannabinoids are present in specific amounts, and wherein the consumption of other drugs is reduced by at least 20% compared with that of administering a product comprising the same cannabinoids amounts and one half the amount of said primary terpene.

10. The method of claim 6,
wherein when said conditions and/or symptoms are selected from the group consisting of anxiety, irritability, nervousness, restless, stress, depression, mood swing and/or affect disorder, said product comprises CBD and optionally THC at CBD to THC weight/weight ratio greater than 1, said primary terpene is selected from the group consisting of limonene, linalool, terpineol, citronellol, eucalyptol, caryophyllene, geraniol, menthol, iso-menthone, germacrene, pinene, citronellol, cardinol, neral, geranial, myrcene, terpinene, menth-2-en-1-ol, humulene, bisabolol, borneol, fenchol, nerolidol and combinations thereof, and said product optionally comprises at least one of linalyl acetate, geranyl acetate, menthol, piperine, eugenol, citronellyl formate, jasmone, benzy acetate, benzyl alcohol and a herbal extract selected from the group consisting of extracts of avena sativa, bacopa monnieri, centella (Hydrocotyl) asiatica, humulus lupulus, hypericum perforatum, melissa officinalis, passiflora incarnata, valeriana officinalis and combinations thereof;
wherein when said conditions and/or symptoms are selected from the group consisting of difficulty in concentrating, brain fog and/or memory loss, said product comprises CBD and optionally THC at CBD to THC weight/weight ratio greater than 1, said primary terpene is selected from the group consisting of eucalyptol, pinene, carene, limonene, pulegone, terpineol, cymene, linalool, caryophyllene and combinations thereof, and said product optionally comprises at least one herbal extract selected from the group consisting of extracts of asphalentum ponjabianum—mumio, bacopa monnieri, centella (hydrocotyl) asiatica, eleutherococcus senticosus, ginkgo biloba, lepidium meyenii, lycium barbarum, paullinia cupana, rhodiola rosea and combinations thereof;
wherein when said conditions and/or symptoms comprise general discomfort, said primary terpene is selected from the group consisting of limonene, linalool, humulene, terpinene, borneol, caryophyllene and combinations thereof;
wherein when said conditions and/or symptoms are selected from the group consisting of fatigue and/or intermittent dizziness, said product comprises CBD and optionally THC at CBD to THC weight/weight ratio greater than 1, said primary terpene is selected from the group consisting of pinene, limonene, terpinene, eucalyptol, myrcene, terpineol, linalool, caryophyllene and combinations thereof, and said product optionally comprises at least one herbal extract selected from the group consisting of extracts of eleutherococcus senticosus, ganoderma lucidum, grifola frondosa, lentinula edodes, lepidium meyenii, panax ginseng, rhodiola rosea and combinations thereof; or
wherein when said conditions and/or symptoms are selected from the group consisting of reduced libido and/or sexual dysfunction, said primary terpene is selected from the group consisting of limonene, linalool, terpineol, geraniol, eucalyptol, pinene, citronellol and combinations thereof, and said product optionally comprises at least one herbal extract selected from the group consisting of extracts of epimedium grandiflorum, ganoderma lucidum, lepidium meyenii, turnera diffusa, vitex agnus castus, withania somnifera and combinations thereof.

11. The method of claim 6,
wherein when said conditions and/or symptoms are selected from the group consisting of headache and/or migraine, said primary terpene is selected from the group consisting of myrcene, linalool, caryophyllene, eucalyptol, limonene, nerolidol, fenchol, humulene, guaiol, borneol, pinene, bisabolol, terpinene, terpineol and combinations thereof, and said product optionally comprises at least one herbal extract selected from the group consisting of extracts of angelica sinensis, corydalis yanhusuo, piscidia erythrina, rosmarinus officinalis, stachys betonica, tanacetum parthenium, verbena officinalis and combinations thereof;
wherein when said conditions and/or symptoms are selected from the group consisting of breast pain and/or Mastalgia, said primary terpene is selected from the group consisting of pinene, limonene, caryophyllene, myrcene, linalool, geraniol, eucalyptol, terpineol, sabinene and combinations thereof;
wherein when said conditions and/or symptoms are selected from the group consisting of arthralgia and/or arthritis, said primary terpene is selected from the group consisting of myrcene, linalool, guaiol, caryophyllene, eucalyptol, pinene, nerolidol, sabinene, limonene, terpinene, menth-2-en-1-ol, geraniol, guaiol, humulene, bisabolol, borneol, fenchol, terpineol and combinations thereof, and said product optionally comprises at least one of linalyl acetate, geranyl acetate, eugenol, methylis-oeugenol, menthol, eugenyl acetate, piperine and a herbal extract selected from the group consisting of extracts of boswellia spp, capsicum spp, corydalis yanhusuo, harpagophytum procumbens, piscidia erythrina, salix alba, zingiber officinalis and combinations thereof; or
wherein when said conditions and/or symptoms are selected from the group consisting of muscle tension and/or myalgia, said primary terpene is selected from the group consisting of myrcene, pinene, nerolidol, linalool, humulene, caryophyllene, limonene, bisabolol and combinations thereof, and said product optionally comprises at least one herbal extract selected from the group consisting of extracts of boswellia spp, salix alba, valeriana edulis, viburnum opulus, chamaemelum and combinations thereof.

12. The method of claim 6,
wherein when said conditions and/or symptoms comprise weight gain, said product comprises CBD and optionally THC at CBD to THC weight/weight ratio greater than 1, said primary terpene is selected from the group consisting of eucalyptol, caryophyllene, limonene, humulene, phytol and combinations thereof, and said product optionally comprises at least one herbal extract selected from the group consisting of extracts of ephedra sinica, foeniculum vulgare, fucus vesiculosus, garcinia cambogia, gymnema sylvestre, laminaria spp, paullinia cupana and combinations thereof;
wherein when said conditions and/or symptoms are selected from the group consisting of digestive problems, intestinal disorders, abdominal pain (colic), nausea, constipation and/or diarrhea, said primary terpene is selected from the group consisting of limonene, cymene, terpinene, caryophyllene, citronellol, sabinene, myrcene, linalool, germacrene, geraniol, pinene, terpineol and combinations thereof, and said product optionally comprises piperine;
wherein when said conditions and/or symptoms are selected from the group consisting of inflammation, oxidative stress and/or paresthesia, said primary terpene is selected from the group consisting of caryophyllene, pinene, myrcene, limonene, humulene, citronellol, eucalyptol, linalool, beta amyrin, cycloartenol, terpinene, terpineol, geraniol, carvacrol, thymol, cardinol, terpinene, menth-2-en-1-ol, carotol, humulene, nerolidol and combinations thereof, and said product optionally comprises at least one of linalyl acetate, geranyl acetate, eugenol, eugenyl acetate, piperine and combinations thereof;
wherein when said conditions and/or symptoms are selected from the group consisting of cardiovascular disease, palpitation and tachycardia, said primary terpene is selected from the group consisting of linalool, camphene, limonene, pinene, borneol and combinations thereof, and said product optionally comprises at least one herbal extract selected from the group consisting of extracts of cimicifuga (actaea) racemosa, crataegus spp, leonurus spp, passiflora incarnata and combinations thereof; or
wherein when said conditions and/or symptoms comprise pre-eclampsia or toxemia, said primary terpene is selected from the group consisting of limonene, neral, geranial, caryophyllene, linalool, myrcene, nerolidol, fenchol, humulene, guaiol, borneol, cymene, terpinene and combinations thereof, and said product optionally comprises at least one herbal extract selected from the group consisting of extracts of Crataegus spp, olea europea, passiflora incarnata and combinations thereof.

13. The method of claim 6,
wherein when said conditions and/or symptoms are selected from the group consisting of dysmenorrhea, pelvic pain, spasms, cramps, labor contraction and/or endometriosis, said primary terpene is selected from the group consisting of myrcene, linalool, caryophyllene, eucalyptol, nerolidol, limonene, nerolidol, sabinene, humulene, terpineol, citronellol, geraniol, pinene, limonene, camphene, terpinene, borneol, camphor, menthone, iso-menthone, carvacrol, thymol, carene, nerol, farnesol, menth-2-en-1-ol and combinations thereof, and said product optionally comprises at least one of linalyl acetate, geranyl acetate, eugenol, methylis-oeugenol, menthol, eugenyl acetate, piperine, chamazulene, citronllyl formate, benzyl acetate, benzyl alcohol and a herbal extract selected from the group consisting of extracts of cimicifuga (actaea) racemosa, paeonia lactiflora, viburnum opulus, viburnum opulus, vitex agnus castus and combinations thereof;
wherein when said conditions and/or symptoms comprise pain and/or cramps, said primary terpene is selected from the group consisting of myrcene, linalool, caryophyllene, eucalyptol, nerolidol, sabinene, humulene, terpineol, citronellol, geraniol, pinene, limonene, camphene, terpinene, borneol, camphor, menthone, iso-menthone, carvacrol, thymol, carene, nerol, farnesol, menth-2-en-1-ol, guaiol and combinations thereof, and said product optionally comprises at least one herbal extract selected from the group consisting of extracts of angelica sinensis, piscidia erythrina, tanacetum parthenium, viburnum opulus, boswellia spp, capsicum spp, corydalis yanhusuo, harpagophytum procumbens, rosmarinus officinalis, salix alba, zingiber officinalis and combinations thereof;
wherein when said conditions and/or symptoms are selected from the group consisting of premenstrual syndrome (PMS), premenstrual tension, and/or premenstrual dysphoric disorder (PMDD), said primary terpene is selected from the group consisting of limonene, linalool, eucalyptol, nerolidol, sabinene, pinene, caryophyllene, terpineol, geraniol, thujone, camphor, viridiflorol, champhene, myrcene, caryophyllene, terpinene, carene, cirtonellol, carotol, neral, geranial and combinations thereof, and said product optionally comprises at least one of linalyl acetate, geranyl acetate, citronellyl formate, menthol, eugenol piperine, jasmone and a herbal extract selected from the group consisting of extracts of angelica sinensis, cimicifuga (actaea) racemosa, dioscorea villosa spp, paeonia lactiflora, vitex agnus castus and combinations thereof;
wherein when said conditions and/or symptoms are associated with hormonal dysfunction and/or imbalance and/or fertility, said primary terpene is selected from the group consisting of linalool, terpineol, geraniol, thujone, camphor, viridiflorol, borneol, champhene, pinene, eucalyptol, caryophyllene, germacrene, pinene, carene, cirtonellol, nerol, farnesol, limonene, myrcene, terpinene, menth-2-en-1-ol, carotol, sabinene, neral, geranial, nerolidol, terpineol, carene and combinations thereof, and said product optionally comprises at least one of linalyl acetate, geranyl acetate, chamazulene, citronellyl formate and a herbal extract selected from the group consisting of extracts of angelica sinensis, chamaelirium lutea, cimicifuga (actaea) racemosa, dioscorea villosa spp., trifolium pratense, vitex agnus castus and combinations thereof; or
wherein when said conditions and/or symptoms are selected from the group consisting of Polycystic Ovary Syndrome (POCS), amenorrhea, oligomenorrhea, hypermenorrhea and/or metrorrhagia, said primary terpene is selected from the group consisting of humulene, linalool, terpineol, caryophyllene, caryophyllene oxide, terpinene, citronellol, geraniol, thujone, camphor, viridiflorol, pinene, camphene, sabinene, myrcene, eucalyptol, carene, carotol, limonene, sabinene, cardinal, pinene and combinations thereof, and said product optionally comprises at least one of linalyl acetate, geranyl acetate, eugenol, chamazulene, citronellyl formate and a herbal extract selected from extracts of angelica sinensis, cimicifuga (actaea) racemosa, dioscorea villosa spp., vitex agnus castus, humulus lupulus, hypericum perforatum, lavendula officinalis, melissa officinalis, passiflora incarnata, valeriana officinalis and combinations thereof.

14. The method of claim 6,
wherein when said conditions and/or symptoms are associated with perimenopause and/or menopause, said primary terpene is selected from the group consisting of caryophyllene, caryophyllene oxide, carene, linalool, myrcene, terpinene, terpineol, citronellol, eucalyptol, nerolidol, limonene, geraniol, menthol, iso-menthone, germacrene, pinene, cardinol, neral, geranial, menth-2-en-1-ol, pulegone, cymene, fenchol, humulene, guaiol, borneol, carene, sabinene, myrtenol, camphene, nerol, farnesol, carotol and combinations thereof, and aid product optionally comprises at least one herbal extract selected from the group consisting of extracts of angelica sinensis, anemarrhenae asphodeloides, cimicifuga (actaea) racemosa, glycyrrhiza glabra. hypericum perforatum, panax quinquefolium, trifolium pratense, avena sativa, cimicifuga (actaea) racemosa, humulus lupulus, hypericum perforatum, lavendula officinalis, melissa officinalis, passiflora incarnata, valeriana officinalis and combinations thereof;
wherein when said conditions and/or symptoms comprise osteoporosis, said product comprises CBD and optionally THC at CBD to THC weight/weight ratio greater than 1, said primary terpene is selected from the group consisting of caryophyllene, caryophyllene oxide, carene, eucalyptol, sabinene, pinene, limonene, borneol, thymol, camphor and combinations thereof, and said product optionally comprises at least one herbal extract selected from the group consisting of extracts of angelica sinensis, cimicifuga (Actaea) racemosa, dioscorea villosa, trifolium pratense and combinations thereof;
wherein when said conditions and/or symptoms comprise sleep disorder and/or insomnia, said primary terpene is selected from the group consisting of linalool, caryophyllene, myrcene, terpineol, terpinene, citronellol, eucalyptol, nerolidol, sabinene, pinene, limonene, borneol, fenchol, bisabolol humulene, phytol and combinations thereof, and said product optionally comprises at least one of linalyl acetate, chamazulene and a herbal extract selected from the group consisting of extracts of avena sativa, cimicifuga (actaea) racemosa, humulus lupulus, hypericum perforatum, lavendula officinalis, melissa officinalis, passiflora incarnata, valeriana officinalis and combinations thereof;
wherein when said conditions and/or symptoms are selected from the group consisting of menopausal skin, itchy skin, age-related wrinkles and/or stretch marks, wherein said product comprises CBD and optionally THC at CBD to THC weight/weight ratio greater than 1, said primary terpene is selected from the group consisting of linalool limonene, nerolidol, pinene terpineol, myrcene, geraniol, nerol, citronellol, farnesol, carotol and combinations thereof, and said product optionally comprises at least one of linalyl acetate, citronellyl formate, eugenol, benzy acetate, benzyl alcohol and a herbal extract selected from the group consisting of extracts of glycyrrhiza glabra, plantago spp, symphytum officinalis, trifolium pratense, trigonella foenum graecum, ulmus rubra/fulva, verbascum thapsus and combinations thereof;
wherein when said conditions and/or symptoms are selected from the group consisting of hair loss and/or thinning, said primary terpene is selected from the group consisting of linalool, terpineol, eucalyptol, borneol, camphor, pinene, geraniol and combinations thereof, and said product optionally comprises at least one herbal extract selected from the group consisting of extracts of extracts of rehmania glutinosa preparata, rosmarinus officinalis, serenoa serrulata, urtica dioica and combinations thereof;
wherein when said conditions and/or symptoms comprise brittle nails, said primary terpene is selected from the group consisting of linalool, eucalyptol, pinene, limonene and combinations thereof;
wherein when said conditions and/or symptoms comprise irregular body odor, said primary terpene is selected from the group consisting of linalool, terpineol and combinations thereof; or
wherein when said conditions and/or symptoms are selected from the group consisting of hot flashes, facial flushing, night sweat, irritability, palpitation and/or tachycardia, said primary terpene is selected from the group consisting of caryophyllene, myrcene, humulene, linalool, terpineol, geraniol, pinene, carene, citronellol, limonene, eucalyptol, sabinene, myrtenol, terpinene, champhene, borneol and combinations thereof, and said product optionally comprises at least one of linalyl acetate, geranyl acetate, citronellyl formate, and a herbal extract selected from the group consisting of extracts of angelica sinensis, anemarrhenae asphodeloides, cimicifuga (actaea) racemosa, dioscorea villosa, salvia officinalis, salvia officinalis, rehmania glutinosa preparata and combinations thereof.

15. The method of claim 6,
wherein when conditions and/or symptoms are selected from the group consisting of female urogenital and/or reproductive system infection and/or disorder, said primary terpene is selected from the group consisting of myrcene, humulene, caryophyllene, linalool, terpineol, geraniol, pinene, germacrene, cirtonellol, limonene, myrcene, terpinene, menth-2-en-1-ol, borneol, eucalyptol, myrtenol, cymene, caryophyllene oxide, ocimene, nerolidol, camphor, sabinene, thujone, camphor, viridiflorol, menthol, iso-menthone, caryophyllene, germacrene, geraniol, nerol, neral, geranial, citronellol, carvacol, thymol, carene, myrtenol, carene, thujone and combinations thereof, and said product optionally comprises at least one of linalyl acetate, geranyl acetate, eugenol, eugenyl acetate, piperine, menthol, citronellyl formate and a herbal extract selected from the group consisting of extracts of apium graveolens, dioscorea villosa spp, althea officinalis, anemarrhenae asphodeloides, anemarrhenae asphodeloides, apium graveolens, arctostaphylos uva-ursi, barosma betulina, coptis chinensis, echinacea spp, equisitum arvense, hydrastis canadensis, plantago spp, serenoa serrulata, uncaria tomentosa, urtica dioica, viola tricolor, zea mays, cimicifuga (actaea) racemosa and combinations thereof;
wherein when said conditions and/or symptoms comprise urine incontinence and/or lower urinary tract disorder, said primary terpene is selected from the group consisting of linalool, terpineol, pinene, carene, geraniol, thujone, camphor, viridiflorol, caryophyllene, germacrene, cirtonellol, eucalyptol, myrtenol, sanatalol, sabinene, limonene and combinations thereof, and said product optionally comprises at least one of linalyl acetate, geranyl acetate, citronellyl formate and a herbal extract selected from the group consisting of extracts of barosma betulina, equisitum arvense, plantago spp, urtica dioicanettle and combinations thereof;
wherein when said conditions and/or symptoms comprisevaginal dryness, said primary terpene is selected from the group consisting of myrcene, humulene, caryophyllene, linalool, terpineol, geraniol, pinene, germacrene, cirtonellol, limonene, myrcene, terpinene, menth-2-en-1-ol, eucalyptol, myrtenol and combinations thereof, and said product optionally comprises at least one of linalyl acetate, geranyl acetate, citronellyl formate and a herbal extract selected from the group consisting of extracts of dioscorea villosa spp, vitex agnus castus, withania somnifera, schisandra chinensis and combinations thereof;
wherein when said conditions and/or symptoms are selected from the group consisting of fibroid, myomas and cysts in the female genital system, said primary terpene is selected from the group consisting of linalool, terpineol, limonene, pinene, caryophyllene, caryophyllene oxide, terpinene, citronellol and combinations thereof, and said product optionally comprises at least one herbal extract selected from the group consisting of extracts of angellica sinesis, larrea mexicana, viburnum prunifolium, vitex agnus castus, trifolium and combinations thereof; or
wherein when said conditions and/or symptoms are selected from the group consisting of cystitis, urethritis and pyelonephritis, said primary terpene is selected from the group consisting of thujone, pinene, limonene, cymene, caryophyllene, caryophyllene oxide, eucalyptol, terpineol, sabinene, linalool, cardinol and combinations thereof, and said product optionally comprises at least one of eugenol and a herbal extract selected from the group consisting of extracts of althea officinalis, andrographis paniculata, anemarrhenae asphodeloides, apium graveolens, arctostaphylos uva-ursi, barosma betulina, coptis chinensis, echinacea spp, hydrastis canadensis, plantago spp, uncaria tomentosa, zea mays and combinations thereof.

16. The method of claim 3, comprising
administering to said subject for a first period of time a first said product comprising a first cannabinoid at a first cannabinoid amount and a first primary terpene at a first primary terpene amount, followed by
a. administering to said subject for a second period of time said product comprising said first cannabinoid at said first cannabinoid amount or less and a second primary terpene at a second primary terpene amount;
b. administering to said subject for a second period of time said product comprising said first cannabinoid at said first cannabinoid amount or less and a first primary terpene at an increased first primary terpene amount;
c. administering to said subject for a second period of time said product comprising said first cannabinoid at a said first cannabinoid amount or less, first primary terpene at a first primary terpene amount and at least one secondary terpene; and/or
d. administering to said subject for a second period of time said product comprising said first cannabinoid at a said first cannabinoid amount or less, first primary terpene at a first primary terpene amount and a second primary terpene.

17. The method of claim 3 wherein said product is selected from the group consisting of tablets, gel capsules, medical patches, topicals, creams, varnishes, sublingual oils, sprays, edibles, suppositories, tampons, rectal candles, cigarettes, vaporizer liquids, nasal preparations, preparations containing micro and/or nano-emulsions, preparations containing micro and/or nano-particles and combinations thereof.

18. The method of claim 2, wherein said conditions and/or symptoms are selected from the group consisting of hot flashes, facial flushing, night sweat, osteoporosis, fatigue, irritability, insomnia and/or sleep disorder, difficulty in concentration, depression, memory loss, brain fog, headache, migraine, anxiety, nervousness, restless, stress, mood swing, affect disorder, general discomfort, muscle tension, myalgia, intermittent dizziness, paresthesia, indigestion, digestive problems, intestinal disorders, abdominal pain, irritability, palpitations, tachycardia, nausea, constipation, diarrhea, arthralgia and/or arthritis, dysmenorrheal, pelvic pain, spasms, cramps, labor contraction, endometriosis Polycystic Ovary Syndrome (POCS), amenorrhea, oligomenorrhea, hypermenorrhea, metrorrhagia, premenstrual syndrome (PMS), premenstrual tension, premenstrual dysphoric disorder (PMDD), cold hands and feet, weight gain, mental awareness problems, sexual pain disorder, dyspareunia, breast pain and/or mastalgia, menopausal skin, inflammation, oxidative stress, cardiovascular disease, loss of pelvic muscle tone, urinary incontinence, lower urinary tract disorders, vaginal dryness, women urogenital and/or reproductive system infection, fibroid, myomas and/or cysts in the female genital system, human papillomavirus (HPV), Uterine Fibroid, cystitis, urethritis, pyelonephritis, women hormonal dysfunction and/or imbalance, irregular periods, pre-eclampsia toxemia, reduced libido, reduced sexual dysfunction, hair loss and/or thinning, age-related winkles, stretch marks, brittle nails, gam disorders, irregular body odor, conditions and/or symptoms induced by estrogen deficiency and combinations thereof.

19. The method of claim 2, wherein said primary terpene is selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thujone, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

20. A composition comprising five or less terpenes in a concentration of at least 10% of the total terpene content, forming a terpene composition with enhanced therapeutic effect for treating conditions and/or symptoms associated with perimenopause, menopause, female urogenital or reproductive system infections and/or disorders, menstrual cycle, hormonal deficiency and/or hormonal imbalance.
